(19) United States
(12) Patent Application Publication
(10) Pub. No.: US 2016/0018400 A1
(43) Pub. Date: Jan. 21, 2016

(54) DIAGNOSTIC AND PROGNOSTIC BIOMARKERS FOR PROSTATE CANCER AND OTHER DISORDERS

(71) Applicant: CANCER RESEARCH TECHNOLOGY LIMITED, London Greater London (GB)

(72) Inventors: Hayley WHITAKER, Cambridge Cambridgeshire (GB); David NEAL, Cambridge Cambridgeshire (GB)

(21) Appl. No.: 14/773,580

(22) PCT Filed: Mar. 13, 2014
(86) PCT No.: PCT/GB2014/050767

§ 371 Date: Sep. 8, 2015

(30) Foreign Application Priority Data
Mar. 14, 2013 (GB) 1304612.3

(51) Int. Cl. G01N 33/574; C12Q 1/68

(52) U.S. Cl. CPC G01N 33/57434; C12Q 1/6886; G01N 2333/948; G01N 2333/4703; C12Q 2600/158; C12Q 2600/118

(57) ABSTRACT

The present invention relates to the use of VPS28 and/or VPS13A as biomarkers for diagnosing prostate cancer, prostate intraepithelial neoplasia (PIN) or atypical small acinar proliferation (ASAP) and to the use of VPS13A, VPS28 and/or NAALADL2 as biomarkers for predicting the prognosis of prostate cancer. The invention also relates to the use of VPS13A, VPS28 and/or NAALADL2 as biomarkers for determining the grade or pathological stage of prostate cancer and monitoring progression of prostate cancer. In addition, the invention relates to the use of NAALADL2 as a biomarker for diagnosing colon, pancreatic or breast cancer. Assays, systems and storage media based on the use of these biomarkers are also provided.

The present invention relates to the use of VPS28 and/or VPS13A as biomarkers for diagnosing prostate cancer, prostate intraepithelial neoplasia (PIN) or atypical small acinar proliferation (ASAP) and to the use of VPS13A, VPS28 and/or NAALADL2 as biomarkers for predicting the prognosis of prostate cancer. The invention also relates to the use of VPS13A, VPS28 and/or NAALADL2 as biomarkers for determining the grade or pathological stage of prostate cancer and monitoring progression of prostate cancer. In addition, the invention relates to the use of NAALADL2 as a biomarker for diagnosing colon, pancreatic or breast cancer. Assays, systems and storage media based on the use of these biomarkers are also provided.

### BACKGROUND TO THE INVENTION

Prostate cancer is the most common cancer in males in the United Kingdom with an incidence of 135 cases per 100,000 men (source: CR-UK website) and is the most common cancer diagnosed in North American men, excluding skin cancers. It is estimated that in 2012, approximately 241,740 new cases and 28,170 prostate cancer-related deaths will occur in the United States (source: National Cancer Institute website). The introduction of prostate-specific antigen (PSA) testing led to an increase in prostate cancer incidence (source: National Cancer Institute website). However, mortality rates have remained have only marginally decreased. Age, disease stage, Gleason grade and serum PSA are used for risk stratification (source: CR-UK website), but PSA remains the most useful biomarker in prostate cancer with regards to diagnosis and prognosis.

However, PSA is not an ideal biomarker for prostate cancer as PSA can be elevated by a number of benign conditions including benign prostatic hyperplasia (BPH) and prostatitis (Farley, 2010). If a cut-off of 4 ng/mL is used, the sensitivity is 21% and specificity is 91% (Wolf, et al., 2012). Hence, there will be a significant number of patients who will undergo prostate biopsies for detection of prostate cancer unnecessarily.

The biomarker prostate cancer gene 3 (PCA3) is being used increasingly to diagnose prostate cancer (Salagierski and Schalken, 2012). Its sensitivity is around 65% and its specificity is around 60%. However, current assays utilise PCA3 mRNA quantification in urine post-prostatic massage, which makes it a more invasive process.

Using circulating nucleic acids as biomarkers has advantages over proteins, such as their ability to be amplified and detected with high sensitivity and specificity (Schwarzenbach et al., 2011). Expression arrays and real-time PCR allow quantification of many genes in a single experiment. Leon et al. showed over 30 years ago that circulating DNA levels were increased in cancer patients compared to healthy controls (see Leon et al., 1977). Current technological advancements have led to circulating RNA being used in the discovery and development of biomarkers. RNA expression in peripheral blood samples is a new source of potential biomarkers (Papadopoulou et al., 2006) and RNA in blood is likely to reflect the early event in the development of cancer. The PAXgene system is used for the storage and purification of RNA from 2.5 mL of peripheral blood (Rainen et al., 2002). It provides storage of blood samples for 50 months at −20° C. Its use has enabled the investigation of differences between RNA expression levels in patient samples with and without cancer. The PAXgene system has been used in studies investigating peripheral RNA levels in haematological and rheumatological disease (Batliwalla et al., 2005; Lewis et al., 2011). From an oncological perspective, there have been studies in peripheral RNA levels using the PAXgene system in breast and thyroid cancer (Li et al., 2004; Yang et al., 2011).

PIN consists of pre-existing prostatic ducts and acini lined by cytologically atypical cells. The distribution of PIN mirrors the frequency of the zonal predilection for carcinoma of the prostate. The frequency of high grade PIN in needle biopsy series ranges from 5 to 16%. The prevalence of high grade PIN in radical prostatectomy specimens is high; it is present in 85-100% of specimens, reflecting the strong association between the lesion and prostate cancer (Ayala and Ro, 2007). There is epidemiological, morphological and molecular evidence that PIN is a precursor lesion to some carcinomas of the prostate (Montironi et al., 2011). The clinical importance of recognizing PIN is based on its association with PCa. PIN can be identified at low magnification by three important characteristics: (i) a darker lining of the ductal structures; (ii) a lining thicker than the surrounding normal ducts and acini, and (iii) a complex intraluminal pattern of growth (Montironi et al., 2011). However, diagnosis of PIN can be challenging as the central zone glands are architecturally more complex than the peripheral and transition zone glands of the prostate and exhibit a certain degree of nuclear stratification that may be interpreted as PIN (Montironi et al., 2011). In addition, bridging papillary formation with a central vascular core, and focal tubular or cribriform patterns may be present in the normal prostate (Montironi et al., 2011). The central zone is frequently found in core biopsies from the base of the prostate and can make PIN diagnosis difficult (Montironi et al., 2011). The most common forms of prostate invasive ductal adenocarcinoma have also been reported to mimic micropapillary and cribriform high grade PIN, making diagnosis challenging (Montironi et al., 2011).

Atypical small acinar proliferation (ASAP) is a diagnosis that incorporates a continuum ranging from benign, histologically atypical mimics of cancer to marginally sampled cancer (Bostwick and Meiers, 2006; Montironi et al., 2006). A pathologist may also refer to ASAP as a proliferation of usually small acini with features highly suggestive of, but not diagnostic for, carcinoma (Bostwick and Meiers, 2006; Montironi et al., 2006). A prostatic core biopsy showing a focus of ASAP may be suspicious for, but not diagnostic of, cancer (Bostwick and Meiers, 2006; Montironi et al. 2006). ASAP foci are found in approximately 2-5% of prostate needle biopsy specimens and are located most often in the peripheral zone of the prostate; they are rarely located in the transition zone. ASAP suspicious for malignancy discovered after prostatic core biopsy is highly predictive of subsequent prostatic adenocarcinoma on repeat biopsy, with a reported range of 17-60% of cases (Bostwick and Meiers, 2006; Montironi et al., 2006). Schlesinger et al. (2005) found prostatic adenocarcinoma in subsequent biopsies in 23% of cases after prior diagnosis of PIN alone and in 37% after diagnosis of ASAP alone.

BPH is prostate gland enlargement that can cause urinary and other symptoms. Untreated prostate gland enlargement can block the flow of urine out of the bladder and can cause bladder, urinary tract or kidney problems. Prostate gland enlargement rarely causes signs and symptoms in men younger than 40. By 55, about 1 in 4 men have some signs and symptoms and by 75, about half of men report some symptoms (Source: Mayo Clinic website). Having a blood relative such as a father or brother with prostate problems increases risk of BPH development and prostate enlargement is more common in American and Australian men (Source: Mayo Clinic website). BPH is diagnosed most often using a Digital Rectal Examination (DRE) and a measurement of Prostate-Specific Antigen (PSA) (Source: National Kidney and Urologic Diseases Information Clearinghouse (NKUDIC) website). PSA is a protein produced by prostate cells to liquefy semen and is frequently present at elevated levels in the blood of men who have prostate cancer and BPH (Source: National Kidney and Urologic Diseases Information Clearinghouse (NKUDIC) website). The U.S. Food and Drug Administration (FDA) has approved a PSA test for use in conjunction with a digital rectal examination to help detect prostate cancer in men who are age 50 or older and for monitoring men with prostate cancer after treatment. However, the ability of the PSA test to discriminate cancer from BPH, and the best course of action following a finding of elevated PSA, is limited (Source: National Cancer Institute website).

VPS13A has recently been linked to gastric and colorectal cancers as well as chronic obstructive pulmonary disease (Alexandre et al., 2012; An et al., 2012). Mutations in VPS13A have been linked to chorea acanthocytosis, a neurogeneretaive disorder characterised by learning, difficulties, muscle weakness and muscle twitches. Pathologically, chorea acanthocytosis is characterised by spikey red blood cells suggesting actin polymerisation may be altered in individuals with VPS13A mutations and this is consistent with findings in neuronal cells (Foller et al., 2012; Hayashi et al., 2012).

VPS28 forms part of a large multi-protein ESCRT complex, a highly conserved endosomal sorting complex (Pineda-Molina et al., 2006; Rusten et al., 2012). Endosomes are responsible for co-ordinating vesicular transport between the trans-Golgi network, plasma membrane and lysosomes. Endocytosis of membrane receptors results in early endosomes which are stratified into recycling endosomes where receptors are returned to the cell surface or into late endosomes and lysosomes where proteins are down regulated (e.g. EGFR). The ESCRT complex consists of a number of proteins (VPS28, VPS23 and VPS37) which are also known to associate with TSG101, a known androgen receptor modifier and coregulator (Burgdorf et al., 2004; Sun et al., 1999). WO 2009/118205 includes VPS28 in a list of possible cancer biomarkers, all derived from indicators of c-myc activity. However, the focus of this reference is lung cancer, and not prostate cancer.

N-Acetylated, alpha-linked acidic dipeptidase like-2 (NAALADL2) is a novel protein member of the N-Acetylated, alpha-linkedacidicdipeptidase (NAALADase) protein family which all have glutamate carboxypeptidase activity (Stauch et al., 1989). The NAALAD family are also similar to prostate specific membrane antigen (PSMA), a known prostate biomarker being investigated for imaging and drug targeting in prostate cancer (Liu et al., 2012; Osbourne et al., 2012). The rs17531088 risk allele in NAALADL2 has previously been linked to Kawasaki disease which affects the blood vessels and can lead to death (Burgner et al., 2009). The NAALADL2 gene was also identified as the site of a breakpoint leading to Cornelia de Lange syndrome, a rare developmental malformation syndrome characterised by mental handicap, growth retardation, distinctive facial features and limb reduction defects (Tonkin et al., 2004). WO 2009/028521 refers to the use of NAALADL2 for prostate cancer diagnosis, especially hormone refractory disease, and treatment. However, utility of NAALADL2 in prostate cancer prognosis, staging of disease, or monitoring of disease progression is not exemplified, claimed, or described in WO 2009/028521, but underpins several aspects of the present application.

The inventors aimed to identify a diagnostic and prognostic target gene set for prostate cancer and PIN using circulating RNA through expression array analysis, qPCR validation, and correlation with expression array analysis in prostate tissue from the Taylor-Sawyers dataset (Osbourne et al., 2012). They also investigated whether there is correlation of gene expression at the circulating RNA level and in prostate tissue with corresponding protein expression in prostate tissue. This was assessed using immunohistochemistry of core biopsy specimens and tissue microarray (TMA).

As a result of these analyses, the inventors identified diagnostic and prognostic biomarkers for prostate cancer, PIN and ASAP that are able to distinguish between these conditions and benign prostatic hyperplasia (BPH), unlike PSA. This increases the specificity of assays performed using these biomarkers.

### STATEMENTS OF INVENTION

In a first aspect, the invention provides a method for diagnosing prostate cancer, PIN or ASAP in a subject, said method comprising determining whether a test sample obtained from the subject expresses:
(i) a gene encoding a VPS13A protein and/or a gene encoding a VPS28 protein; or
(ii) a VPS13A protein and/or a VPS28 protein;
   at a level higher than the expression of the respective gene(s) or protein(s) in a normal reference sample, wherein a higher level of expression and/or activity of the respective gene(s) or protein(s) in the test sample compared to the normal reference sample is indicative of the presence of prostate cancer, PIN or ASAP in the subject.

The method may comprise determining whether the test sample obtained from the subject expresses:
(i) a gene encoding a VPS13A protein and a gene encoding a VPS28 protein; or
(ii) a VPS13A protein and a VPS28 protein;
   at a level higher than the expression of the respective genes or proteins in a normal reference sample, wherein a higher level of expression and/or activity of the respective genes or proteins in the test sample compared to the normal reference sample is indicative of the presence of prostate cancer, PIN or ASAP in the subject.

The method may comprise determining whether a test sample obtained from the subject expresses:
(i) a gene encoding a VPS28 protein and a gene encoding an NAALADL2 protein; or
(ii) a VPS28 protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the presence of prostate cancer, PIN or ASAP in the subject.

The method may comprise determining whether the test sample obtained from the subject expresses:
(i) a gene encoding a VPS13A protein and a gene encoding an NAALADL2 protein; or
(ii) a VPS13A protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the presence of prostate cancer, PIN or ASAP in the subject.

The method may comprise determining whether the test sample obtained from the subject expresses:
(i) a gene encoding a VPS13A protein, a gene encoding a VPS28 protein and a gene encoding an NAALADL2 protein; or
(ii) a VPS13A protein, a VPS28 protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the presence of prostate cancer, PIN or ASAP in the subject.

In a second aspect, the invention provides a method for determining the grade of prostate cancer in a subject, said method comprising detecting whether a test sample obtained from the subject expresses:
(i) a gene encoding a VPS13A protein, a gene encoding a VPS28 protein and/or a gene encoding a NAALADL2 protein; or
(ii) a VPS13A protein, a VPS28 protein and/or a NAALADL2 protein;
   at a level higher than the expression of the respective gene(s) or protein(s) in a normal reference sample, wherein the level of expression and/or activity of the respective gene(s) or protein(s) in the test sample compared to the normal reference sample is indicative of the grade of prostate cancer in the subject. Higher expression and/or activity of the respective gene(s) or protein(s) is indicative of a higher grade of prostate cancer in the subject.

The method may comprise determining whether the test sample obtained from the subject expresses:
(i) a gene encoding a VPS13A protein and a gene encoding a VPS28 protein; or
(ii) a VPS13A protein and a VPS28 protein;
   at a level higher than the expression of the respective genes or proteins in a normal reference sample, wherein a higher level of expression and/or activity of the respective genes or proteins in the test sample compared to the normal reference sample is indicative of the grade of prostate cancer in the subject.

The method may comprise determining whether the test sample obtained from the subject expresses:
(i) a gene encoding a VPS28 protein and a gene encoding an NAALADL2 protein; or
(ii) a VPS28 protein and an NAALADL2 protein;
   at a level higher than the expression of the respective genes or proteins in a normal reference sample, wherein a higher level of expression and/or activity of the respective genes or proteins in the test sample compared to the normal reference sample is indicative of the grade of prostate cancer in the subject.

The method may comprise determining whether the test sample obtained from the subject expresses:
(i) a gene encoding a VPS13A protein and a gene encoding an NAALADL2 protein; or
(ii) a VPS13A protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the grade of prostate cancer in the subject.

The method may comprise determining whether the test sample obtained from the subject expresses:
(i) a gene encoding a VPS13A protein, a gene encoding a VPS28 protein and a gene encoding an NAALADL2 protein; or
(ii) a VPS13A protein, a VPS28 protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the grade of prostate cancer in the subject.

A test sample obtained from a subject that expresses:
(i) a gene encoding a VPS13A protein, a gene encoding a VPS28 protein and/or a gene encoding a NAALADL2 protein; or
(ii) a VPS13A protein, a VPS28 protein and/or a NAALADL2 protein;
   at a level higher than the expression of the respective gene(s) or protein(s) in a normal reference sample indicates that the subject is likely to have prostate cancer with a Gleason grade of at least 3+3. For example, the subject is likely to have prostate cancer with a Gleason grade of 3+4 or 4+3. Most likely, the subject has prostate cancer with a Gleason grade of 4+3.

In a third aspect, the invention provides a method for determining the pathological stage of prostate cancer in a subject, said method comprising detecting whether a test sample obtained from the subject expresses:
(i) a gene encoding a VPS13A protein, a gene encoding a VPS28 protein and/or a gene encoding a NAALADL2 protein; or
(ii) a VPS13A protein, a VPS28 protein, and/or a NAALADL2 protein;
   at a level higher than the expression of the respective gene(s) or protein(s) in a normal reference sample, wherein a higher level of expression and/or activity of the respective genes or proteins in the test sample compared to the normal reference sample is indicative of the pathological stage of prostate cancer in the subject. Higher expression and/or activity of the respective gene(s) or protein(s) is indicative of a higher pathological stage of prostate cancer in the subject.

The method may comprise determining whether the test sample obtained from the subject expresses:
(i) a gene encoding a VPS13A protein and a gene encoding a VPS28 protein; or
(ii) a VPS13A protein and a VPS28 protein;
   at a level higher than the expression of the respective genes or proteins in a normal reference sample, wherein a higher level of expression and/or activity of the respective genes or proteins in the test sample compared to the normal reference sample is indicative of the pathological stage of prostate cancer in the subject.

The method may comprise determining whether the test sample obtained from the subject expresses:
(i) a gene encoding a VPS28 protein and a gene encoding an NAALADL2 protein; or
(ii) a VPS28 protein and an NAALADL2 protein;
   at a level higher than the expression of the respective genes or proteins in a normal reference sample, wherein a higher level of expression and/or activity of the respective genes or proteins in the test sample compared to the normal reference sample is indicative of the pathological stage of prostate cancer in the subject.

The method may comprise determining whether the test sample obtained from the subject expresses:
(i) a gene encoding a VPS13A protein and a gene encoding an NAALADL2 protein; or
(ii) a VPS13A protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the pathological stage of prostate cancer in the subject.

The method may comprise determining whether the test sample obtained from the subject expresses:
(i) a gene encoding a VPS13A protein, a gene encoding a VPS28 protein and a gene encoding an NAALADL2 protein; or
(ii) a VPS13A protein, a VPS28 protein and a NAALADL2 protein;
   at a level higher than the expression of the respective genes or proteins in a normal reference sample, wherein a higher level of expression and/or activity of the respective genes or proteins in the test sample compared to the normal reference sample is indicative of the pathological stage of prostate cancer in the subject.

A test sample obtained from a subject that expresses:
(i) a gene encoding a VPS13A protein, a gene encoding a VPS28 protein and/or a gene encoding a NAALADL2 protein; or
(ii) a VPS13A protein, a VPS28 protein and/or a NAALADL2 protein;
   at a level higher than the expression of the respective gene(s) or protein(s) in a normal reference sample indicates that the subject is likely to have prostate cancer with a pathological stage of pT2 or pT3.

In a fourth aspect, the invention provides a method for monitoring progression of prostate cancer in a subject, said method comprising determining whether a test sample obtained from the subject expresses:
(i) a gene encoding a VPS13A protein, a gene encoding a VPS28 protein and/or a gene encoding a NAALADL2 protein; or
(ii) a VPS13A protein, a VPS28 protein and/or a NAALADL2 protein;
   at a level higher than the expression of the respective gene(s) or protein(s) in a previous cell or tissue sample obtained from said subject, wherein a higher level of expression and/or activity of the respective gene(s) or protein(s) in the test sample compared to the previous sample is indicative of progression of prostate cancer to a more aggressive form.

The method may comprise determining whether the test sample obtained from the subject expresses:
(i) a gene encoding a VPS13A protein and a gene encoding a VPS28 protein; or
(ii) a VPS13A protein and a VPS28 protein;
   at a level higher than the expression of the respective genes or proteins in a previous sample obtained from said subject, wherein a higher level of expression and/or activity of the respective genes or proteins in the test sample compared to in the previous sample is indicative of progression of prostate cancer to a more aggressive form.

The method may comprise determining whether the test sample obtained from the subject expresses:
(i) a gene encoding a VPS28 protein and a gene encoding an NAALADL2 protein; or
(ii) a VPS28 protein and an NAALADL2 protein;
   at a level higher than the expression of the respective genes or proteins in a previous sample obtained from said subject, wherein a higher level of expression and/or activity of the respective genes or proteins in the test sample compared to in the previous sample is indicative of progression of prostate cancer to a more aggressive form.

The method may comprise determining whether the test sample obtained from the subject expresses:
(i) a gene encoding a VPS13A protein and a gene encoding an NAALADL2 protein; or
(ii) a VPS13A protein and an NAALADL2 protein;
   at a level higher than the expression of the respective genes or proteins in a previous sample obtained from said subject, wherein a higher level of expression and/or activity of the respective genes or proteins in the test sample compared to in the previous sample is indicative of progression of prostate cancer to a more aggressive form.

The method may comprise determining whether the test sample obtained from the subject comprises a cell or tissue that expresses:
(i) a gene encoding a VPS28 protein, a gene encoding a VPS13A protein and a gene encoding an NAALADL2 protein; or
(ii) a VPS28 protein, a VPS13A protein and an NAALADL2 protein;
   at a level higher than the expression of the respective genes or proteins in a previous cell or tissue sample obtained from said subject, wherein a higher level of expression and/or activity of the respective genes or proteins in the test sample compared to in the previous sample is indicative of progression of prostate cancer to a more aggressive form.
   A test sample comprising a cell or tissue that expresses:
(i) a gene encoding a VPS28 protein and/or a gene encoding a VPS13A protein; a gene encoding a VPS28 protein and a gene encoding an NAALADL2 protein; a gene encoding a VPS13A protein and a gene encoding an NAALADL2 protein; or a gene encoding a VPS28 protein, a gene encoding a VPS13A protein and a gene encoding an NAALADL2 protein; or
(ii) a VPS28 protein and/or a VPS13A protein; a VPS28 protein and an NAALADL2 protein; a VPS13A protein and an NAALADL2 protein; or a VPS28 protein, a VPS13A protein and an NAALADL2 protein;
   at a level higher than the expression of the respective gene(s) or protein(s) in a previous cell or tissue sample obtained from said subject may indicate that the prostate cancer has progressed to a Gleason grade of at least 3+3. For example, the prostate cancer may have progressed to a Gleason grade of 3+4 or 4+3. Most likely, the prostate cancer has progressed to a Gleason grade of 4+3.

In a fifth aspect, the invention provides a method for predicting the prognosis of a subject with prostate cancer, said method comprising detecting whether a test sample obtained from the subject expresses:
(i) a gene encoding a VPS13A protein, a gene encoding a VPS28 protein and/or a gene encoding an NAALADL2 protein; or
(ii) a VPS13A protein, a VPS28 protein and/or an NAALADL2 protein;
   at a level higher than the expression of the respective gene(s) or protein(s) in a normal reference sample, wherein a higher level of expression and/or activity of the respective gene(s) or protein(s) in a normal reference sample compared to in the test sample is indicative of a poor prognosis.

The method may comprise detecting whether the test sample obtained from the subject expresses:
(i) a gene encoding a VPS13A protein and a gene encoding a VPS28 protein; a gene encoding a VPS28 protein and an NAALADL2 protein; a gene encoding a VPS13A protein and an NAALADL2 protein; or a gene encoding a VPS13A protein, a gene encoding a VPS28 protein and a gene encoding an NAALADL2 protein; or
(ii) a VPS13A protein and a VPS28 protein; a VPS28 protein and an NAALADL2 protein; a VPS13A protein and an NAALADL2 protein; or a VPS13A protein, a VPS28 protein and an NAALADL2 protein;
   at a level higher than the expression of the respective genes or proteins in a normal reference sample, wherein detection of said cell or tissue is indicative of a poor prognosis.

In a sixth aspect, the invention provides an assay comprising the steps of:

(i) measuring or quantifying expression of a gene encoding a VPS13A protein, a gene encoding a VPS28 protein and/or a gene encoding a NAALADL2 protein, or expression and/or activity of a VPS13A protein, a VPS28 protein and/or a NAALADL2 protein in a test sample obtained from a subject; and
(ii) comparing the measured or quantified expression and/or activity of the respective gene(s) or protein(s) with their expression in a normal reference sample or in a previous sample obtained from the subject, and if the expression of the respective gene(s) or protein(s) is increased relative to their respective expression and/or activity in the normal reference sample or the previous sample, identifying the subject as having an increased probability of having prostate cancer, PIN or ASAP, or a more aggressive form of prostate cancer.

In a seventh aspect, the invention provides an assay for selecting a treatment or further testing regimen for a subject suspected of having prostate cancer, the assay comprising measuring or quantifying expression of a gene encoding a VPS13A protein, a gene encoding a VPS28 protein and/or a gene encoding a NAALADL2 protein, or expression and/or activity of a VPS13A protein, a VPS28 protein and/or a NAALADL2 protein in a test sample obtained from a subject and comparing the expression of the respective gene(s) or protein(s) with their expression and/or activity in a normal reference sample or in a previous sample obtained from the subject to determine whether the subject requires further testing (e.g. a biopsy), surgery (e.g. a radical prosectatomy), radiotherapy and/or chemotherapy.

In an eighth aspect, the invention provides a system for obtaining data from at least one test sample obtained from at least one subject, wherein the system comprises:

(i) a measuring module quantifying expression of a gene encoding a VPS13A protein, a gene encoding a VPS28 protein and/or a gene encoding a NAALADL2 protein, or expression and/or activity of a VPS13A protein, a VPS28 protein and/or a NAALADL2 protein in a test sample obtained from a subject;
(ii) a storage module configured to store data output from the measuring module;
(iii) a comparison module adapted to compare the data stored on the storage module with a reference and/or control data obtained from a normal reference sample or from a previous sample obtained from said subject, and to provide a comparison content; and
(iv) an output module for displaying the comparison content for the user, and if the expression and/or activity of the respective gene(s) or protein(s) is higher than the reference and/or control data obtained from the normal reference sample or the previous sample, then identifying the subject as likely to have prostate cancer, PIN or ASAP, or to have a more aggressive form of prostate cancer.

In a ninth aspect, the invention provides a computer-implemented system to facilitate the diagnosis of prostate cancer, PIN or ASAP and/or monitor progression of prostate cancer in a subject, the system comprising:

(i) a determination module configured to receive and output expression of a gene encoding a VPS13A protein, a gene encoding a VPS28 protein and/or a gene encoding a NAALADL2 protein; or expression and/or activity of a VPS13A protein, a VPS28 protein and/or a NAALADL2 protein;
(ii) a storage module configured to store output data from the determination module;
(iii) a comparison module adapted to compare the output data stored on the storage module with a reference and/or control data from a normal reference sample or a previous sample obtained from the subject, and to provide a comparison content; and
(iv) an output module for displaying the comparison content for the user, wherein if the expression and/or activity of the respective gene(s) or protein(s) is higher than the reference and/or control data obtained from the normal reference sample or from the previous sample, then the subject is likely to have prostate cancer, PIN or ASAP, or to have a more aggressive form of prostate cancer.

In a tenth aspect, the invention provides a computer readable storage medium comprising:

(i) a storing data module containing data from a subject that represents expression of a gene encoding a VPS13A protein, a gene encoding a VPS28 protein and/or a gene encoding a NAALADL2 protein; or expression and/or activity of a VPS13A protein, a VPS28 protein and/or a NAALADL2 protein;
(ii) a comparison module that compares the data stored on the storing data module with reference data and/or control data from a normal reference sample or from a previous sample from the subject, and provides a comparison content; and
(iii) an output module displaying the comparison content for the user, wherein if the expression and/or activity of the respective gene(s) or protein(s) is higher than the reference and/or control data obtained from the normal reference sample, then the subject is likely to have prostate cancer, PIN or ASAP, or to have a more aggressive form of prostate cancer.

In an eleventh aspect, the invention provides a method for diagnosing colon, pancreas or breast cancer in a subject, said method comprising determining whether a test sample obtained from the subject expresses:
(i) a gene encoding a NAALADL2 protein; or
(ii) a NAALADL2 protein;
   at a level higher than the expression of the gene encoding a NAALADL2 protein or the NAALADL2 protein in a normal reference sample, wherein a higher level of expression and/or activity of the gene encoding a NAALADL2 protein or the NAALADL2 protein in the test sample compared to the normal reference sample is indicative of the presence of colon, pancreatic or breast cancer in the subject.

The following statements apply to any of the above aspects or embodiments of the invention.

In any of the methods, assays, systems or storage media of the invention disclosed herein, the subject is preferably a human.

The test sample is preferably whole blood, plasma, urine, ejaculate, stool, a pancreatic biopsy, a prostate biopsy (e.g. a prostate fine needle biopsy), tissue from a radical prostatectomy, cyst fluid or biliary pancreatic sponge.

The normal reference sample may include benign or normal cells or tissue from the subject. In some embodiments, the normal reference sample may be taken from the same tissue as the test sample. For example, the normal reference sample may be an internal reference present in the test sample. In some embodiments, the normal reference sample may be a corresponding sample type from a healthy subject, i.e. a subject without prostate cancer, PIN or ASAP. For example, the normal reference sample may be whole blood, plasma, urine or ejaculate taken from a healthy subject, i.e. a subject without prostate cancer, PIN or ASAP. In the method for monitoring the progression of prostate cancer in a subject, the previous sample is preferably of the same type as the test sample.

The step of determining expression of a gene encoding a VPS28 protein, a VPS13A protein and/or an NAALADL2 protein may, for example, be carried out by determining expression of VPS28, VPS13A or NAALADL2 mRNA. For example, expression of VPS28, VPS13A or NAALADL2 mRNA may be determined by quantitative RT-PCR, digital PCR, next generation sequencing (NGS) or northern blotting.

The step of determining expression of a VPS28, VPS13A and/or NAALADL2 protein may, for example, be carried out by detecting the VPS28, VPS13A and/or NAALADL2 protein with an antibody that binds to the relevant protein. For example, expression of VPS28, VPS13A and/or NAALADL2 protein may be determined by immunohistochemistry, enzyme-linked immunosorbent assay (ELISA), western blotting, flow cytometry, multiplexing, e.g. by multiplexed ELISA, or monoclonal antibody imaging modalities and related cell surface targeted technology (e.g. nano-spotting).

Expression of a VPS28, VPS13A and/or NAALADL2 protein may be determined by determining the activity of the VPS28, VPS13A and/or NAALADL2 protein. For example, enzymatic activity of the NAALADL2 protein may be determined.

Each of the methods, assays or systems of the invention may include the step of obtaining the test sample from the subject. Each of the methods, assays or systems may also include the step of processing the test sample to obtain DNA, cDNA, mRNA and/or protein.

Each of the methods, assays or systems of the invention may include the step of measuring (i) expression of a gene encoding a VPS13A protein, a gene encoding a VPS28 protein and/or a gene encoding an NAALADL2 protein; or (ii) expression and/or activity of a VPS13A protein, a VPS28 protein and/or a NAALADL2 protein.

Each of the methods, assays or systems of the invention may include an additional step of selecting a subject identified as having prostate cancer, PIN or ASAP for treatment, or treating a subject identified as having prostate cancer, PIN or ASAP. For example, the subject may be selected for or given surgery (e.g. a radical prostatectomy), chemotherapy and/or radiotherapy.

For low risk disease (PSA<10, organ confined disease) surgery, watchful waiting, active surveillance, radiotherapy, brachytherapy, chemotherapy are all options. Patients are less likely to be offered watchful waiting, active surveillance or brachytherapy with intermediate risk disease (PSA>10, organ confined disease) as recurrence is more likely. If the markers described herein can predict who is likely to do well/badly this decision could be better informed. Patients with high risk disease (PSA>10, locally advanced) would be offered surgery, radiotherapy or chemotherapy but again decisions might be influenced by the increased risk of recurrence inferred by a biomarker. Relapsed patients are normally offered radiotherapy in the first instance. This or chemotherapy could be offered post surgery if a high risk of recurrence was predicted.

Each of the methods, assays or systems of the invention may also include an additional step of further testing the subject identified as likely to have prostate cancer, PIN or ASAP, or selecting the subject identified as likely to have prostate cancer, PIN or ASAP for further testing. For example, a biopsy sample may be taken from the subject or the subject may be selected for a biopsy. If the test sample obtained from the patient was tissue or cells from a pancreatic biopsy, then the subject identified from this test sample as likely to have prostate cancer, PIN or ASAP may be re-biopsied or selected for a re-biopsy.

These and other aspects of the invention are described in further detail below.

### BRIEF DESCRIPTION OF THE FIGURES

FIG. 1 shows a prostate tissue microarray (TMA) core stained for VPS13A. Tumour glands stain heavily for VPS13A, while benign glands do not stain at all (black arrowheads). Nuclei are counterstained with haematoxylin. Staining is punctate and largely apically distributed. At high power, a proportion of tissues also exhibited ring-like structures (white arrowheads).

FIG. 2 shows expression of VPS13A in a Cambridge TMA determined by immunohistochemistry and stratified by Gleason grade (G3-G5). Each patient had a minimum of 3 benign and 6 tumour cores from two regions, as well as up to 3 PIN containing cores (which were frequently benign). Where clear benign and tumour was detected in a single core, both regions were scored independently.

FIG. 3 shows expression of VPS13A in a Trans-Atlantic Prostate Group (TAPG) TMA determined by IHC in patients with a Gleason grade of less than or equal to 6, seven, or greater than or equal to 8. Each core was given a single score.

FIG. 4 shows expression of VPS13A in a Karolinksa TMA determined by IHC. Each patient had 3 benign and 3 tumour cores assessed. Staining intensity and spread were measured to give the immunoreactivity product (IRP). P-values were calculated using a Mann-Whitney 2-tailed t-test.

FIG. 5 shows Kaplan-Meier estimates of recurrence free survival by categorised immunoreactivity product of VPS13A staining (IRP). Dashed lines indicate 5 year survival.

FIG. 6 shows VPS13A staining of the hormone refractory (HR) TMA. There was no statistical difference between matched hormone naïve (HN) and HR tissue (p=0.49), but VPS13A could distinguish from benign (p<0.0001).

FIG. 7 shows the relative expression of VPS13A mRNA in various grades of prostate cancer. mRNA from whole blood collected in PAXgene tubes was assayed for expression of circulating VPS13A mRNA. Levels rose significantly in aggressive disease before dropping in advance disease. Grouped data were analysed using a 1-way ANOVA with a Kruskal-Wallis correction. Pairwise comparison of Gleason 3+4 and 4+3 disease was performed using a Mann-Whitney paired t-test. All results are expressed relative to the mean benign result.

FIG. 8 shows the relative expression of VPS13A in metastatic patients. Circulating mRNA from the whole blood of metastatic patients was assayed for expression of circulating VPS13A mRNA. Pairwise comparisons were performed using a Mann-Whitney paired t-test. All results are expressed relative to the mean hormone naïve result.

FIG. 9 shows the association between VPS13A vesicles and lysosomes. VPS13A vesicles were stained and lysosomes were stained with LAMP2. Co-localisation events are shown with white arrowheads.

FIG. 10 shows the effect of bafilomycin treatment on VPS13A vesicle integration into the lysosomal membrane. Following bafilomycin treatment, VPS13A vesicles integrate with the lysosome membrane stained with LAMP2. There is no evidence of dispersal of VPS13A throughout the lysosomal membrane.

FIG. 11 shows the effect of VPS13A knockdown on PSA secretion. When VPS13A is knocked down to 60-70% of endogenous levels, the secretion of PSA is significantly reduced.

FIG. 12 shows the effect of treatment with 1,2-bis(o-aminophenoxy)ethane-N,N,N′,N′-tetraacetic acid (BAPTA) or calcimycin on VPS13A protein expression. Non-targeting control LNCaP cells or siVPS13A cells were treated for 8 hours with 10 μM BAPTA (B), calcimycin (Cal) or control (C). Protein lysates were separated by SDS-PAGE and probed for VPS13A and tubulin.

FIG. 13 the effect of treatment with BAPTA and calcimycin on fusion between VPS13A vesicles and lysosomes. LNCaP cells were treated with either 100 mM calcimycin, a calcium ionophose, or 10 nM BAPTA, a calcium chelator, for 4 hours. The cells were then fixed and stained for LAMP2 and VPS13A. Using the 100× lens, the number of lysosomes in 10 fields of view were counted. Then the number of VPS13A fusion events was counted in the same field of view. A fusion event was classed as a VPS13A vesicle touching or integrated into a LAMP2 positive lysosome. *Data are shown normalised to the number of lysosomes in each field of view. P-values were calculated using a Mann-Whitney 2-tailed t-test.

FIG. 14 shows prostate tissue stained for VPS28. Tumour glands stain heavily for VPS28 (black arrowheads) while benign glands do not stain at all (white arrowheads). Staining is punctate and largely perinuclear.

FIG. 15 shows expression of VPS28 in the Cambridge TMA determined by IHC and stratified by Gleason grade (G3-G5). Each patient had a minimum of 3 benign and 6 tumour cores from 2 regions. Where clear benign and tumour was detected in a single core, both regions were scored independently.

FIG. 16 shows expression of VPS28 in the Karolinska TMA determined by IHC. Each patient had 3 benign and 3 tumour cores assessed. Staining intensity and spread were measured to give the immunoreactivity product (IRP). P-values are calculated using a Mann-Whitney 2-tailed t-test.

FIG. 17 shows Kaplan-Meier estimates of recurrence free survival by categorised immunoreactivity product of VPS28 staining (IRP). Dashed lines indicate 5 year survival.

FIG. 18 shows the relative expression of VPS28 mRNA in various grades of prostate cancer. mRNA from whole blood was collected in PAXgene tubes and assayed for expression of circulating VPS28 mRNA. Levels rose significantly in aggressive disease before dropping in advanced disease. Grouped data were analysed using a 1-way ANOVA with a Kruskal-Wallis correction. Pairwise comparison of Gleason 3+4 and 4+3 disease was performed using a Mann-Whitney paired t-test. All results are expressed relative to the mean benign result.

FIG. 19 shows the relative expression of VPS28 mRNA in metastatic prostate cancer patients. Circulating mRNA from the whole blood of metastatic patients was assayed for expression of circulating VPS28 mRNA. Pairwise comparisons were performed using a Mann-Whitney paired t-test. All results are expressed relative to the mean hormone naïve result.

FIG. 20 shows prostate tissue stained for NAALADL2 and PSMA. Tumour glands stain heavily for NAALADL2 along the basal membrane (brown) (black arrowheads) while benign glands do not stain at all (white arrowheads). PSMA stains the apical/luminal membrane (grey arrowheads).

FIG. 21 shows expression of NAALADL2 in the Cambridge TMA determined by IHV and stratified by Gleason grade (G3-G5). Each patient had a minimum of 3 benign and 6 tumour cores from 2 regions. Where clear benign and tumour was detected in a single core, both regions were scored independently.

FIG. 22 shows expression of NAALADL2 in the Cambridge TMA determined by IHC and stratified by pathological stage. Each patient had a minimum of 3 benign and 6 tumour cores from 2 regions. Where clear benign and tumour was detected in a single core, both regions were scored independently.

FIG. 23 shows expression of NAALADL2 in the Karolinska TMA determined by IHC. Each patient had 3 benign and 3 tumour cores assessed and staining intensity and spread measured to give the immunoreactivity product (IRP). P-values are calculated using a Mann-Whitney 2-tailed t-test.

FIG. 24 shows Kaplan-Meier estimates of recurrence free survival by categorised immunoreactivity product of NAALADL2 staining (IRP). Dashed lines indicate 5 year survival.

FIG. 25 shows NAALADL2 staining of hormone refractory (HR) TMA. There was no statistical difference between matched hormone naïve (HN) and HR tissue (p=0.59), but NAALADL2 could distinguish all tumour from benign (p<0.0001).

FIG. 26 shows relative expression of NAALADL2 in prostate cancer patients with different Gleason scores. mRNA from whole blood was collected in PAXgene tubes and assayed for expression of circulating NAALADL2 mRNA. Levels rose significantly in aggressive disease before dropping in advanced disease. Grouped data were analysed using a 1-way ANOVA with a Krustal-Wallis correction, pairwise comparison of Gleason 3+4 and 4+3 disease was performed using a Mann-Whitney paired t-test. All results are expressed relative to the mean benign result.

FIG. 27 shows the relative expression of NAALADL2 in metastatic patients. Circulating mRNA from the whole blood of metastatic patients was assayed for expression of circulating NAALADL2 mRNA. Pairwise comparisons were performed using a Mann-Whitney paired t-test. All results are expressed relative to the mean hormone naïve result.

### DETAILED DESCRIPTION

The present invention is based on the finding that VPS13A, VPS28 and NAALADL2 show increased expression in prostate cancer tissue and can be used as diagnostic and prognostic biomarkers for prostate cancer, PIN and ASAP. The inventors have found that these biomarkers are able to distinguish between different grades and pathological stages of prostate cancer and can be used to monitor the progression to more aggressive forms of the disease. As disclosed herein, these biomarkers may also be used to predict the prognosis of patients with prostate cancer. The inventors have also found that NAALADL2 may also be used as diagnostic biomarker for colon, pancreatic or breast cancer.

The nucleotide and amino acid sequences of human VPS13A, human VPS28 and human NAALADL2 are shown below. VPS13A is also known as CHAC and KIAA0986. VPS28 is also known as vacuolar protein sorting-associated protein 28 homolog, H-Vps28, ESCRT-1 complex subunit VPS 28 and yeast class E protein Vps28p homolog. NAALADL2 is also known as inactive N-acetylated-alpha-linked acidic dipeptidase-like protein 2, NAALADase L2, N-acetylated alpha-linked acidic dipeptidase 2 and glutamate carboxypeptidase II-type non-peptidase homologue.

As described above, the invention relates to a method for diagnosing prostate cancer, PIN or ASAP in a subject, a method for determining the grade or pathological stage of prostate cancer in a subject, a method for monitoring progression of prostate cancer in a subject, or to a method for predicting the prognosis of a subject with prostate cancer. Assays, systems and storage media are also provided.

In the method for diagnosing prostate cancer, PIN or ASAP in a subject, increased expression and/or activity of:
(i) a gene encoding a VPS13A protein and/or a gene encoding a VPS28 protein, or
(ii) a VPS13A protein and/or a VPS28 protein
   in a test sample obtained from the subject compared to expression of the respective gene(s) and/protein(s) in a normal reference sample indicates that the subject is likely to have prostate cancer, PIN or ASAP.

As shown herein, VPS13A and VPS28 expression is not increased in subjects with BPH and therefore, this method can distinguish between the presence of prostate cancer/PIN/ASAP and BPH.

Therefore, the invention also provides a method for diagnosing BPH, the method comprising determining whether a test sample obtained from the subject expresses PSA, but not VPS13A, VPS28 and/or NAALADL2, at a level higher than the expression of the respective gene(s) or protein(s) in a normal reference sample, wherein a higher level of expression of PSA, but not VPS13A, VPS28 and/or NAALADL2, in the test sample compared to the normal reference sample is indicative of the presence of BPH in the subject. Being able to diagnose BPH in this way means that a subject suspected of having prostate cancer is less likely to be over-treated.

In the method for determining the grade of prostate cancer in a subject, increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a normal reference sample is indicative of the grade of prostate cancer in the subject.

Expression and/or activity of one or more of the respective genes or proteins is indicative of the grade of prostate cancer, such that a higher level of expression and/or activity of one or more of the respective genes or proteins is indicative of a higher grade of prostate cancer.

For example, increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a normal reference sample may indicate that the subject is likely to have prostate cancer with a Gleason grade of at least 3+3. For example, the subject is likely to have prostate cancer with a Gleason grade of 3+4 or 4+3. Most likely, the subject has prostate cancer with a Gleason grade of 4+3.

The Gleason grande of a tumour is typically determined by a pathologist by microscopic examination of a tissue biopsy. The pathologist assigns a grade to the most common tumour pattern and a second grade to the next most common tumour patterns. The two grades are added together to produce a Gleason score (see Epstein et al., 2005). The patterns are described below:

### Pattern 1:

Circumscribed nodule of closely-packed but separate, uniform, rounded to oval medium-sized acini (larger glands than pattern 3).

### Pattern 2:

Like Pattern 1, fairly circumscribed, yet at the edge of the tumour nodule there may be minimal infiltration. Glands are more loosely arranged and not quite as uniform as Gleason pattern 1.

### Pattern 3:

Discrete glandular units; typically smaller glands then seen in Gleason pattern 1 or 2. Infiltrates in and amongst non-neoplastic prostate acini. Marked variation in size and shape. Smoothly circumscribed small cribriform nodules of tumour.

### Pattern 4:

Fused microacinar glands; ill-defined glands with poorly formed glandular lumina; large cribriform glands; cribriform glands with an irregular border; hypernephromatoid.

### Pattern 5:

Essentially no glandular differentiation, composed of solid sheets, cords, or single cells; comedocarcinoma with central necrosis surrounded by papillary cribriform, or solid masses.

In the method for determining the pathological stage of prostate cancer in a subject, increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a normal reference sample is indicative of the pathological stage of prostate cancer in the subject.

Expression of one or more of the respective genes or expression and/or activity of one or more of the respective proteins is indicative of the pathological stage of prostate cancer, such that a higher level of expression and/or activity of one or more of the respective genes or proteins is indicative of a higher pathological stage of prostate cancer.

For example, increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a normal reference sample may indicate that the subject is likely to have prostate cancer with a pathological stage of at least pT2 or pT3.

Details of the TNM pathological staging system are provided below (taken from the 6^{th} Edition of the AJCC Cancer Staging Manual, 2002 and the 6^{th} Edition of the UICC Classification of Malignant Tumours).

### Evaluation of the (Primary) Tumour (‘T’)

TX: cannot evaluate the primary tumour
T0: no evidence of tumour
T1: tumour present, but not detectable clinically or with imaging
   T1a: tumour was incidentally found in less than 5% of prostate tissues resected (for other reasons)
   T1b: tumour was incidentally found in greater than 5% of prostate tissue resected
   T1c: tumour was found in a needle biopsy performed due to an elevated serum PSA
T2: the tumour can be felt (palpated) on examination, but has not spread outside the prostate
   T2a: the tumour is in half or less half of one of the prostate gland's two lobes
   T2b: the tumour is in more then half of one lobe, but not both
   T2c: the tumour is in both lobes
T3: the tumour spread through the prostatic capsule (if it is only part-way through, it is still T2)
   T3a: the tumour has spread through the capsule on one or both sides
   T3b: the tumour has invaded on or both seminal vesicles
T4: the tumour has invaded other nearby structures

### Evaluation of the Regional Lymph Nodes (‘N’)

NX: cannot evaluate the regional lymph nodes
N0: there has been no spread to the regional lymph nodes
N1: there has been spread to the regional lymph nodes

### Evaluation of Distant Metastasis (‘M’)

MX: cannot evaluate distant metastasis
M0: there is no distant metastasis
M1: there is distant metastasis
   M1a: the cancer has spread to the lymph noted beyond the regional ones
   M1b: the cancer has spread to the bone
   M1c: the cancer has spread to other sites (regardless of bone involvement)

In the method for monitoring progression of prostate cancer in a subject, increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a previous sample obtained from said subject is indicative of progression of prostate cancer to a more aggressive form.

Increased expression and/or activity of:
(i) a gene encoding a VPS13A protein, a gene encoding a VPS28 protein and/or a gene encoding a NAALADL2 protein, or
(ii) a VPS13A protein, a VPS28 protein and/or a NAALADL2 protein
   in a test sample obtained from the subject compared to a previous cell or tissue sample obtained from said subject may indicate that the prostate cancer has progressed to a Gleason grade of at least 3+3. For example, the prostate cancer may have progressed to a Gleason grade of 3+4 or to a Gleason grade of 4+3. Most likely, the prostate cancer has progressed to a Gleason grade of 4+3. In this way, the method is able to indicate progression of prostate tissue from normal to having a Gleason grade of 3+3, from having a Gleason grade of 3+3 to having a Gleason grade of 3+4 or from having a Gleason grade of 3+4 to having a Gleason grade of 4+3.

In the method for predicting the prognosis of a subject with prostate cancer, increased expression and/or activity of:
(i) a gene encoding a VPS13A protein, a gene encoding a VPS28 protein and/or a gene encoding a NAALADL2 protein, or
(ii) a VPS13A, a VPS28 protein and/or a NAALADL2 protein
   in a test sample obtained from the subject compared to expression and/or activity of the respective gene(s) and/or protein(s) in a normal reference sample is indicative of a poor prognosis.

For example, increased expression and/or activity of one or more of the respective genes or proteins may be predictive of a decreased progression free survival time.

Increased expression and/or activity of one or more of the respective genes or proteins may be indicative of an increased likelihood of clinical or biochemical relapse following radical prostatectomy. This is independent of hormone status.

In the methods, assays or systems of the invention, the subject is preferably a mammal. More preferably, the subject is a human. Most preferably, the subject is a human male. For example, the subject may be a human male who is at least 40 years old. The subject has preferably had no prior treatment for prostate cancer, e.g. no prior radiotherapy.

The methods, systems or assays of the invention include the step of determining VPS13A, VPS28, and/or NAALADL2 expression and/or activity in a test sample obtained from the subject. The test sample is preferably whole blood, plasma, urine, ejaculate, stool, tissue or cells from a pancreatic biopsy, tissue or cells from a radical prostatectomy or a biliary pancreatic sponge.

Blood may be collected as whole blood, serum, plasma-EDTA, plasma-citrate or plasma heparin. Circulating RNA may be collected using a PAXgene tube. Tissue may be collected via biopsy (TRUSP, template, saturation or another method where tissue is collected via a needle) or radical surgery either open or robotic. Urine may be collected and may either be kept ‘whole’ or separated into urinary sediment and supernatant and either could be tested.

Each of the methods, assays or systems of the invention may also include the step of processing the test sample to obtain DNA, cDNA, mRNA and/or protein. For example, DNA, cDNA, mRNA and/or protein can be obtained using commercial kits (e.g. Qiagen™ kits) or making chemical solutions to precipitate DNA, RNA or protein from a biological fluid.

In the methods, assays or systems of the invention, expression of VPS13A, VPS28 and/or NAALADL2 in the test sample may be determined at the mRNA level or at the protein level. For example, expression of the VPS13A, VPS28 and/or NAALADL2 genes may be determined by detecting the levels of VPS13A, VPS28 and/or NAALADL2 mRNA respectively. For example, VPS13A, VPS28 and/or NAALADL2 mRNA may be obtained from whole blood obtained from the subject. Expression of VPS13A, VPS28 and/or NAALADL2 at the protein level may be determined by detecting the expression and/or activity of VPS13A, VPS28 and/or NAALADL2 protein respectively.

Expression of VPS13A, VPS28 and/or NAALADL2 mRNA may be determined by any method known to one skilled in the art. For example, levels of VPS13A, VPS28 and/or NAALADL2 mRNA may be determined by quantitative RT-PCR, digital PCR, next generation sequencing or northern blotting. Each of these methods is well known to one skilled in the art. For example, expression of VPS13A, VPS28 and/or NAALADL2 mRNA may be determined using an array, gene chip or gene set comprising one or more polynucleotides capable of specifically hybridising to VPS13A, VPS28 and/or NAALADL2. Next generation sequencing and/or polynucleotides capable of specifically hybridising to VPS13A, VPS28 and/or NAALADL2 may be used to detect deletions or mutations in the genes encoding VPS13A, VPS28 and/or NAALADL2 protein. The following pre-made Applied Biosystems® primer/probe sets may be used to detect mRNA expression of VPS13A, VPS28 and/or NAALADL2: Hs00362891 m1 (VPS13A), Hs00211938 m1 (VPS28) and Hs00822484 m1 (NAALADL2).

Expression of one or more housekeeping genes, such as rp12 or ribosomal 18S, may also be determined in order to control for the amount of mRNA present in the test sample.

Expression of VPS13A, VPS28 and/or NAALADL2 protein may be determined by any method known to one skilled in the art. For example, levels of VPS13A, VPS28 and NAALADL2 protein may be determined by immunohistochemistry, ELISA detection, western blotting, flow cytometry, multiplexing (e.g. multiplexed ELISA), or monoclonal antibody imaging modalities and related cell surface targeted technology (e.g. nano-spotting). Expression of VPS13A, VPS28 and/or NAALADL2 protein may also be determined by imaging. For example, NAALADL2 is a transmembrane protein and therefore, its expression can be determined by imaging methods, e.g. by detecting NAALADL2 protein using an antibody directed against the extracellular domain of NAALADL2. Expression of VPS13A, VPS28 and/or NAALADL2 protein may also be determined by determining the activity of VPS13A, VPS28 and NAALADL2 protein. For example, the enzymatic activity of NAALADL2 may be determined. Expression of VPS13A, VPS28 and/or NAALADL2 may be determined using the following antibodies: Human Protein Atlas 012413 and R&D Systems AF4665 both recognise NAALADL2, Human Protein Atlas 021662 recognises VPS13A, and Human Protein Atlas 024745 and Santa Cruz sc-30179 both detect VPS28.

Activity of VPS13A, VPS28 and/or NAALADL2 protein may be determined by any method known to one skilled in the art. For example, activity of NAALADL2 may be determined by assaying its enzymatic activity.

The methods of the invention include the step of determining whether the test sample expresses (i) a gene encoding a VPS13A protein, a gene encoding a VPS28 protein and/or a gene encoding an NAALADL2 protein, or (ii) a VPS13A protein, a VPS28 protein and/or an NAALADL2 protein at a level higher than the expression of the respective gene(s) or protein(s) in a normal reference sample. The methods of the invention may also include the step of determining the activity of a VPS13A protein, a VPS28 protein and/or an NAALADL2 protein in the test sample compared to the activity of the respective protein(s) in a normal reference sample.

Examples of suitable normal reference samples for use in the methods, assays or systems of the invention include benign or normal cells or tissue from the subject. In some embodiments, the normal reference sample may be taken from the same tissue as the test sample. For example, the normal reference sample may be an internal reference present in the test sample, such as normal, benign cells present in the test sample. In some embodiments, the normal reference sample may be a corresponding sample type from a healthy subject, i.e. a subject without prostate cancer, PIN or ASAP. For example, the normal reference sample may be whole blood, plasma, urine or ejaculate taken from a healthy subject, i.e. a subject without prostate cancer, PIN or ASAP. In the method for monitoring the progression of prostate cancer in a subject, the previous sample is preferably of the same type as the test sample.

In the method for monitoring the progression of prostate cancer or PIN in a subject, expression and/or activity of VPS13A, VPS28 and/or NAALADL2 in a test sample is compared to the expression and/or activity of the respective gene(s) or protein(s) in a previous sample obtained from said subject. The previous sample for use in the methods, assays and systems of the invention is preferably of the same type as the test sample. The previous sample may have been obtained at least one month, at least two months, at least three months, at least six months, at least one year, at least two years or at least three years earlier than the test sample. Preferably, the same method is used to determine the expression and/or activity of VPS13A, VPS28 and/or NAALADL2 in the test sample as in the previous cell or tissue sample.

In order to be expressed at a level “higher” than the expression of the respective gene(s) or protein(s) in a normal reference sample or in a previous sample, the expression and/or activity of:
(i) a gene encoding a VPS13A protein, a gene encoding a VPS28 protein and/or a gene encoding an NAALADL2 protein, or
(ii) a VPS13A protein, a VPS28 protein and/or an NAALADL2 protein
   in the test sample is preferably at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold or at least 10-fold higher than in the normal reference sample or in the previous sample.

The methods, assays or systems of the invention may include the step of determining the expression of a gene encoding prostate-specific antigen (PSA) or expression of PSA protein in the test sample and calculating the ratio of VPS13A, VPS28 and/or NAALADL2 expression: PSA expression. Expression of PSA represents a surrogate measure of tumour burden and is currently used in the clinic to diagnose prostate cancer. The higher the ratio of VPS13A, VPS28 or NAALADL2 expression: PSA expression, the greater the likelihood that the subject has prostate cancer.

The gene or protein detected in the methods, assays or systems of the invention may be a fragment of a gene encoding a VPS13A, a VPS28, or a NAALADL2 protein or a fragment of a VPS13A, VPS28, NAALADL2 protein.

The gene or protein detected in the methods, assays and systems of the invention may have at least 50%, at least 60%, at least 70%, at least 80%, at least 90%, at least 95%, at least 96%, at least 97%, at least 98%, at least 99% or at least 100% homology with a gene encoding a VPS28, a VPS13A or a NAALADL2 protein, or with a VPS28, a VPS13A or a NAALADL2 protein.

In the methods, assays or systems of the invention, expression and/or activity of any one of VPS13A, VPS28 or NAALADL2 may be determined singly. Alternatively, expression and/or activity of any two or three of these genes or protein may be determined in combination. For example, expression and/or activity of VPS13A and VPS28, VPS28 and NAALAD2, or VPS13A and NAALADL2 may be determined, or expression and/or activity of VPS13A, VPS28 or NAALADL2 may be determined. Preferred combinations include VPS13A and VPS28, and VPS28 and NAALAD2.

In the methods, assays or systems of the invention, expression and/or activity of one or more additional genes or proteins may also be determined. For example, the expression and/or activity of PSA, PCA-3 and MSMB may also be determined. Expression of PSA and PCA-3 has previously been shown to be increased in prostate cancer, while expression of MSMB has been shown to be decreased (Salagierski et al. 2012 and Whitaker et al., 2010).

TMPRSS2-ERG status of the subject may also be determined in the subject in order to stratify the data obtained from the methods, assays and systems of the invention (Salagierski et al. 2012).

Each of the methods, assays or systems of the invention may include the step of obtaining the test sample from the subject. For example, the test sample may be whole blood, plasma, serum, urine, ejaculate, stool, tissue or cells from a pancreatic biopsy, tissue or cells from a radical prostatectomy or a biliary pancreatic sponge. Each of the methods, assays or systems of the invention may also include the step of processing the test sample to obtain DNA, cDNA, mRNA and/or protein. For example, DNA, cDNA, mRNA and/or protein can be obtained using commercial kits (e.g. Qiagen™ kits) or making chemical solutions to precipitate DNA, RNA or protein from a biological fluid.

Each of the methods, assays or systems of the invention may include the step of measuring (i) expression of a gene encoding a VPS13A protein, a gene encoding a VPS28 protein and/or a gene encoding an NAALADL2 protein; or (ii) expression and/or activity of a VPS13A protein, a VPS28 protein and/or a NAALADL2 protein.

Each of the methods, assays or systems of the invention may include an additional step of treating a subject identified as having prostate cancer, PIN or ASAP, or selecting a subject identified as having prostate cancer, PIN or ASAP for treatment. For example, the subject may be given or selected for surgery (e.g. a radical prostatectomy), chemotherapy and/or radiotherapy. A subject identified as having prostate cancer with a Gleason grade of 4+3 is likely to require surgical and/or therapeutic intervention. For example, a patient identified as having prostate cancer with a Gleason grade of 4+3 may be selected for or given surgery (e.g. a radical prostatectomy) and/or radiotherapy. A patient identified as having prostate cancer with a Gleason grade of 4+3 may be selected for or given chemotherapy. A subject identified as having prostate cancer with a pathological stage of pT2 is likely to be selected for or given surgery (e.g. a radical prostatectomy). A patient identified as having prostate cancer with a pathological stage of pT3 is likely to be selected for or given surgery (e.g. a radical prostatectomy) and adjuvant chemotherapy. Such a patient identified as having prostate cancer with a pathological stage of pT3 may also be selected for or given radiotherapy.

Each of the methods, systems or assays of the invention may also include an additional step of further testing the subject identified as likely to have prostate cancer, PIN or ASAP, or selecting the subject identified as likely to have prostate cancer, PIN or ASAP for further testing. For example, a biopsy sample may be taken from the subject, or the subject may be selected for a biopsy. If the test sample obtained from the patient was tissue or cells from a pancreatic biopsy, then the subject identified from this test sample as likely to have prostate cancer, PIN or ASAP may be re-biopsied, or selected for a re-biopsy.

Each of the methods, assays or systems of the invention may include the step of inputting the expression levels and/or activity of VPS13A, VPS28 and/or NAALAD2 obtained from the methods of the invention into a computer database and classifying the subject according to the expression level and/or activity of VPS13A, VPS28 and/or NAALAD2 in the test sample.

Each of the methods, assays or systems of the invention may be used in combination with cell or tissue staining, e.g. haematoxylin and eosin staining, to provide a diagnosis or prognosis or to give additional information about the grade or pathological stage of prostate cancer.

Further aspects and embodiments of the invention will be apparent to those skilled in the art given the present disclosure including the following experimental exemplification.

### Experimental Details

### VPS13A

### Materials and Methods

### Tissue Microarrays (TMAs) and Patient Cohorts

Cambridge TMA—Prostate tissue from radical prostatectomies performed at Addenbrookes Hospital, Cambridge, UK between 2001 and 2005 was used to make tissue microarrays (TMAs) using duplicate 0.6 mm cores taken from paraffin embedded tissue and a Beecher Manual TMA Arrayer (Whitaker et al., 2010). In total, tissue from 32 different patients was used to generate the TMA. Regions of benign or normal prostate (n=4), prostatic intraepithelial neoplasia (PIN) (n=4) and malignancy (n=2-6) were identified by a specialist uro-pathologist (Anne Warren (AW)) for each patient. Malignant tissue was obtained from at least one and, where possible, up to three different tumour foci from each patient. Pathological stage and Gleason grade was confirmed by a specialist uro-pathologist (AW) prior to scoring any IHC staining.

Karolinska TMA—Prostate tissue from radical prostatectomies performed at Karolinska Hospital, Stockholm, Sweden between 1998 and 2002 was used to make tissue microarrays (TMAs) using 3 1 mm cores taken from paraffin embedded tissue and a Beecher Manual Arrayer. In total, tumour tissue from 257 different patients was used to generate the TMA. Malignant tissue was identified and obtained from at least one and, where possible, up to three different tumour foci from each patient. Pathological stage and Gleason grade was confirmed by a specialist uro-pathologist (Lars Egevad) prior to scoring any IHC staining. Benign tissue for each patient was not included in this TMA. Median follow up was 61 months and based upon prostate cancer related deaths. Matched benign and tumour samples were used for the validation of diagnostic utility.

The Trans-Atlantic Prostate Group (TAPG) TMA—Clinicians and scientists from the United States and the United Kingdom have assembled the largest cohort of prostate cancers treated by conservative means with both initial serum PSA levels and centralised Gleason scoring. The detailed methods of cohort assembly have been described in an earlier paper (Cuzick et al., 2006). In short, men were included in this study if they were under 76 years of age at diagnosis and had clinically localised prostate cancer diagnosed between January 1990 and December 1996. Patients who had a radical prostatectomy or radiation therapy within 6 months of diagnosis, or clear evidence of metastatic disease (by bone scan, X-ray, CT scan, MRI, bone biopsy, lymph node biopsy or pelvic lymph node dissection) or clinical indications of metastatic disease (including pathologic fracture, soft tissue metastasis, spinal compression or bone pain) at or within 6 months of diagnosis, were excluded. Eligibility was established by review of patient records by registry data-collection officers and trained medical staff. Clinical staging was centrally reviewed. All patients had centralised Gleason grading by a panel of genitourinary pathologists and had initial diagnostic serum PSA available. Blocks from the trans-urethral resection specimens, which were available, were identified and the corresponding haematoxylin and eosin sections marked for cancerous areas.

These were microarrayed in a series of 24 blocks using 0.6 mm cylinders of tissue. Four cores were taken from different areas of tumour to account for tumour heterogeneity in each case, and areas of adjacent normal tissue were also sampled.

Multi-tumour/normal TMA—A TMA containing 2 tumour and 1 normal core from prostate, oesophagus, liver, thyroid, tongue, soft tissue lymphoma, breast, colon, stomach, tongue, skin, lung, kidney, ovary, uterus, testes, pancreas, thymus was purchased from Stretton Scientific.

Hormone refractory TMA (HR TMA)—Prostate tissue from 75 HR patients (defined as 2 consecutive PSA rises) was made into a TMA. Tissue was obtained from transurethral resection of the prostate performed at Addenbrookes Hospital, Cambridge, UK between 2001 and 2005. Median follow up was 86 months. For the TMA, 0.6 mm cores were taken from paraffin embedded tissue and arrayed using a Beecher Manual TMA Arrayer. Where possible, tumour alone (n=2), mixed tumour and benign (n=2) were obtained for each patient and non-matched benign alone was also included.

### Immunohistochemistry

All immunohistochemistry (IHC) was performed using a Bondmax Autostainer using 1.5M Tris EDTA, pH8.0 for antigen recovery. Anti-VPS13A antibody (Human Protein Atlas) was used at 1:50 and counterstained with DAPI to visualise nuclei.

### Scoring and IHC Data Analysis

For initial qualification using the Cambridge TMA, all regions of each core were scored which often gave rise to multiple scores for adjacent regions in heterogeneous cores. For validation using the TAPG TMA, each core was given a single score based upon the predominant pathology (scoring performed by HW and AW). Cambridge and TAPG TMAs were scored as none (where no staining was present), weak (where staining could be seen but was inconsistent and/or weak), moderate (appreciable staining) or strong (staining could not get any more intense). The Karolinska TMA was scored by Lars Egevad and Amanda Seipel and each core scored by intensity and proportion of cancer cells stained on the scale 0 to 3 independently of each other. Average values of the three intensity and proportion scorings were calculated to give average intensity and proportion values. These values were then multiplied to give the immunoreactivity product (IRP).

Sensitivity, specificity, positive predictive values (PPV) and negative predictive values (NPV) were calculated were possible and results shown. All grouped p-values (n=≧3) were calculated using a 1-way ANOVA with a Kruskal-Wallis correction. All pairwise comparisons were completed using a Mann Whitney 2-tailed t-test.

To generate Kaplan-Meier curves, time-to-event analysis using biochemical recurrence as outcome. Association between immunoreactivity product index and biochemical recurrence was assessed in Cox regression analysis estimating hazard ratios (HR) with corresponding 951 confidence intervals as measured for association. For each protein explored, the immunoreactivity product index was categorized into three groups (0-3, 3-5, and >5) with the lowest category used as reference group. Both crude analysis and analysis adjusted for age, Gleason score, extraprostatic extension, positive surgical margin, vesicle invasion, clinical stage, and preoperative PSA were done.

### PAXgene

2.5 ml of blood was collected from patients in a PAXgene tube and stored according to the manufacturer's instructions. RNA was extracted by Tepnel using PAXgene RNA Blood kit (Qiagen) and subsequently quantified using the Nanodrop ND100. mRNA expression was analysed by qPCR as before (Thirkettle et al., 2009). Primers used in the qPCR are shown in Table 1. 12 samples were collected in each group (benign, Gleason 3+3, 3+4, 4+3, 4+4/4+5) and 11 in the metastatic group. The benign men all had raised PSA but negative biopsy suggesting that a proportion (˜30%) have so far undetected cancers.

The initial metastatic samples came from a range of men with different hormone statuses. A subsequent, more detailed analysis, was completed on a well defined cohort of 12 hormone naïve, 12 hormone relapsed and 11 hormone responsive patients.

### Confocal Microscopy

Cells were fixed and stained as before using anti-VPS13A antibody (1:50, Human Protein Atlas) and LAMP2 antibody (1:500, BD Biosciences) (8). Cells were imaged using Alexafluor 594 and 488 secondary antibodies (Molecular Probes) and mounted with DAPI. All images were obtained using a Nikon Eclipse confocal microscope using a 100× objective. For calcium modifying experiments, LNCaP cells were grown as before to ˜50% confluence before being treated for 8 hours with 10 μM of the calcium ionophore, calcimycin (Invitrogen) or 10 μM of the calcium chelating agent 1,2-bis(o-aminophenoxy)ethane-N,N,N′,N′-tetraacetic acid (BAPTA) (Invitrogen) (Whitaker et al., 2007).

### PSA Measurements

6.67×10⁵ LNCaP cells that had been stably transfected with either an siVPS13A or scrambled control were grown for 72 hours in RPMI media, supplemented with 10% FBS. Media was harvested and centrifuged at 5500 rpm for 5 mins to remove and cellular debris. Supersensitive PSA tests were performed by the Clinical Biochemistry Assay Laboratory, Addenbrookes Hospital. All results were normalised to cell number after 72 hours growth.

### Production of Cell Lysates and Western Blotting

Protein lysates were produced as described (8) and separated by SDS-PAGE before blotting for VPS13A (1:1000, Human Protein Atlas), FAK (1:1000, Cell Signaling Technology), pTyr397-FAK (1:1000, Cell Signaling Technology) or tubulin (1:2000, Abcam).

### Results

### Qualification of VPS13A as a Diagnostic Marker

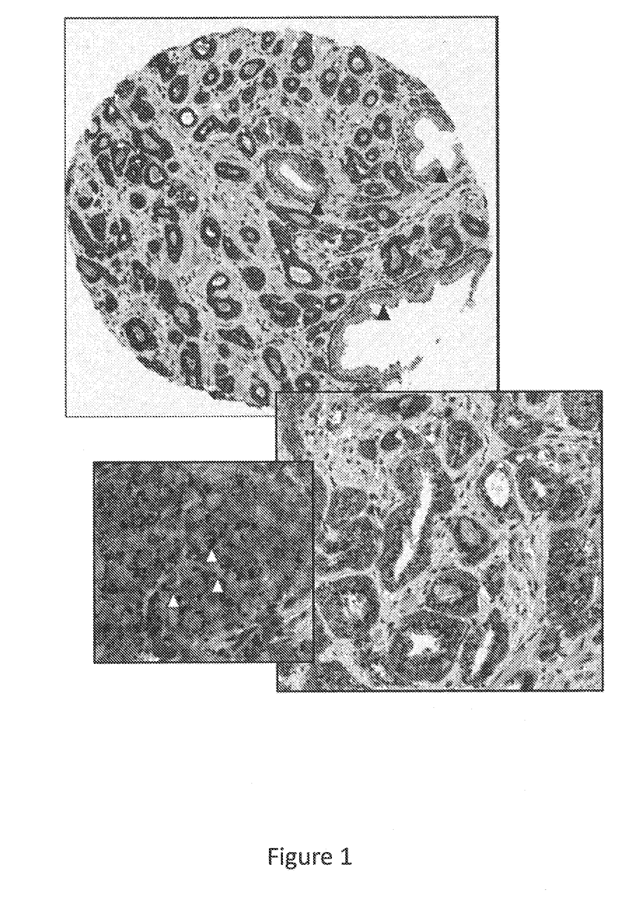

VPS13A expression was assessed using immunohistochemistry (IHC) on prostate tissue. Staining was punctate and distributed towards the apical membrane of luminal epithelial cells, which suggested a possible role in endocytosis. A proportion of tissues also demonstrated relatively large, circular cytoplasmic structures (FIG. 1) suggesting a possible function in phagocytosis or autophagy.

To assess how specific VPS13A may be for prostate tissue, we used a multi-tumour/normal tissue microarray (TMA) comprised of multiple tissue cores from 16 different organs. VPS13A showed some expression in normal kidney, but expression in all other tissues was either low or undetectable.

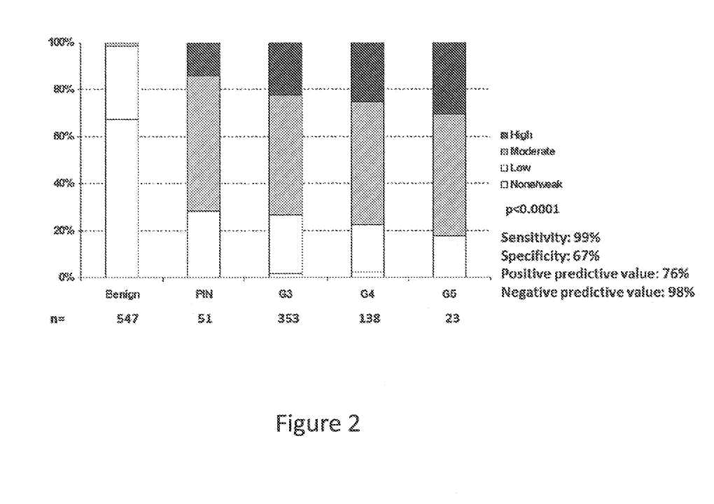

The expression of VPS13A was then determined in the Cambridge TMA comprising 104 patients with multiple benign, prostatic intraepithelial neoplasia (PIN) and tumour regions sampled for each individual. VPS13A was highly significantly up-regulated in PIN and tumours when compared to benign (FIG. 2) (p<0.0001). The positive predictive value (PPV) was 76% and the negative predictive value (NPV) 98%.

### Validation Of VPS13A as a Diagnostic Marker

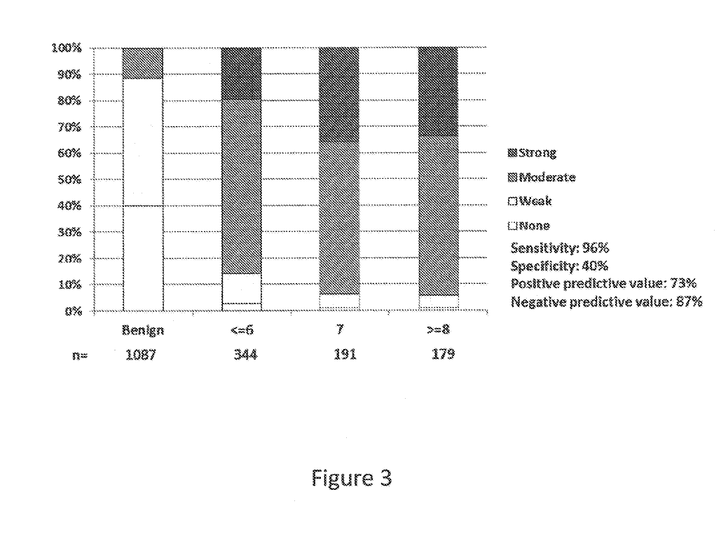

The independent TAPG TMA was used to validate the utility of VPS13A as a diagnostic marker for prostate cancer and demonstrated similar results to the Cambridge TMA with a PPV of 73′ and NPV of 87% (FIG. 3). VPS13A expression was a highly significant diagnostic marker when data was stratified by tumour status or Gleason grade (Tables 2 and 3) (p<0.0001). The VPS13A staining and Gleason grade was available for 709 cancer cores (for univariate analysis and multivariate analysis, the maximum Manual Intensity values in cancer cores were used). Tables 2 and 3 illustrate that VPS13A manual intensity (MI) is highly significantly different in between benign, tumour and PIN (Table 2, p<0.0001) and Gleason score when divided into three categories (<7, 7 and >7, Table 3, p<0.0001). This confirms VPS13A as a useful diagnostic marker.

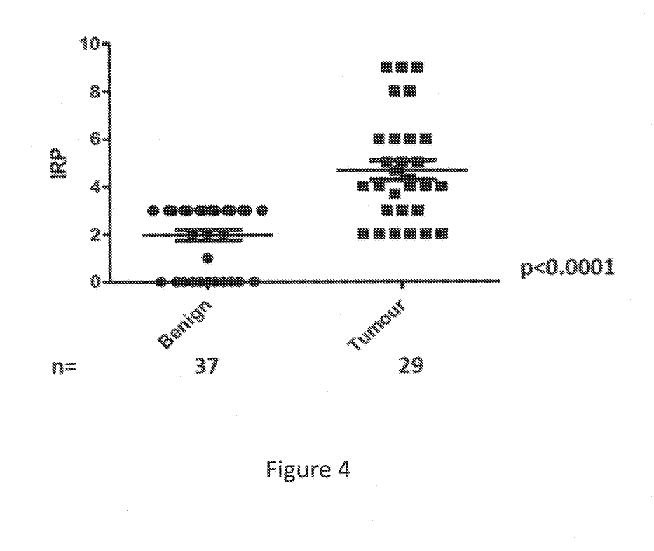

The Karolinska TMA has a limited number of patients with benign as well as tumour cores represented on the TMA. These patients alone were analysed as an additional validation cohort and supported our finding that VPS13A protein expression was significantly different between benign and tumour groups (p<0.0001) (FIG. 4).

### Qualification and Validation of VPS13A as a Predictive Marker

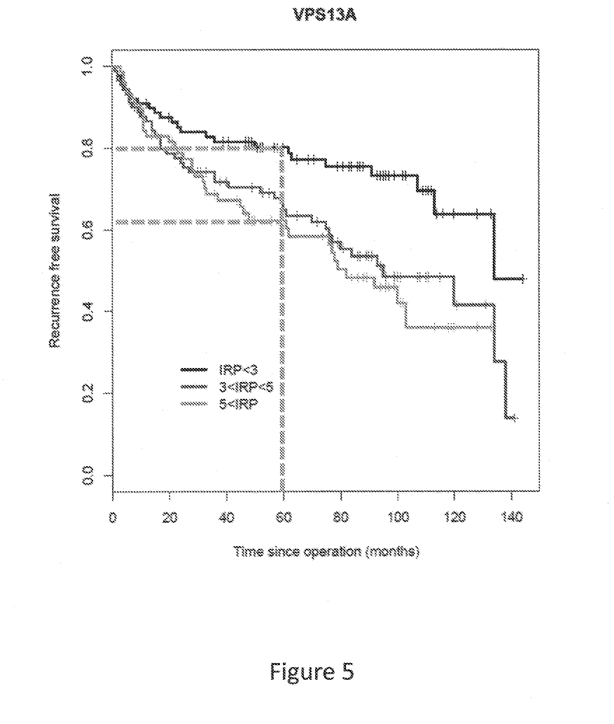

The Karolinska TMA is made from radical prostatectomy specimens with a median of 61 months follow-up which allows analysis of VPS13A as a marker to predict relapse and subsequent death following radical prostatectomy. Taking the immunoreactivity product (IRP) of weak (<3), moderate (>3<5) and strong (>5) VPS13A IHC, Kaplan-Meier curves were generated (FIG. 5). Patients with weak VPS13A expression had a 20% chance of dying from prostate cancer within 5 years whereas patients with moderate and high expression had a 40% chance of relapse and death within 5 years i.e. were twice as likely to die. The hazard ratios show that men with a raised VPS13A (IRP>5) are over twice as likely to relapse and die following radical prostatectomy (p=0.01) (Table 4). The hazard ratio drops to 1.9 and it becomes less significant (p=0.06) when the hazard ratio is adjusted for age, Gleason score, extraprostatic extension, positive surgical margin, vesicle invasion, clinical stage, and preoperative PSA.

The TAPG TMA, made from TURP samples, also has >10 year follow up with death as an endpoint. When the data is split by MI into two groups (0 and 1 versus 2 and 3 or MI of 1 versus 2, 3), VPS13A is borderline significant in a univariate analysis (Table 5). This most likely reflects the method of sample collection compared to the Karolinska samples (radical prostatectomy (Karolinska) versus TURP (TAPG)).

VPS13 does not Predict for Hormone Status

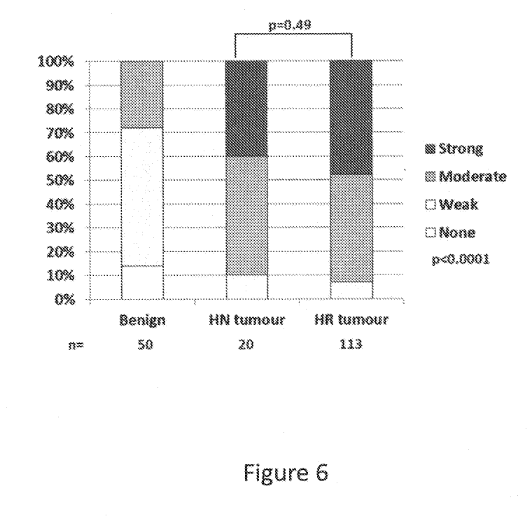

In the LNCaP cell line there was no evidence of androgen regulation of VPS13A. To determine if this was consistent in human tissue we assessed VPS13A expression on the hormone refractory TMA (FIG. 6). Although VPS13A could significantly differentiate between benign and any tumour sample (p<0.001), it was not able to differentiate between hormone naïve (HN) and hormone refractory (HR) tumours (p=0.49).

Circulating VPS13A mRNA as an Alternative Endpoint

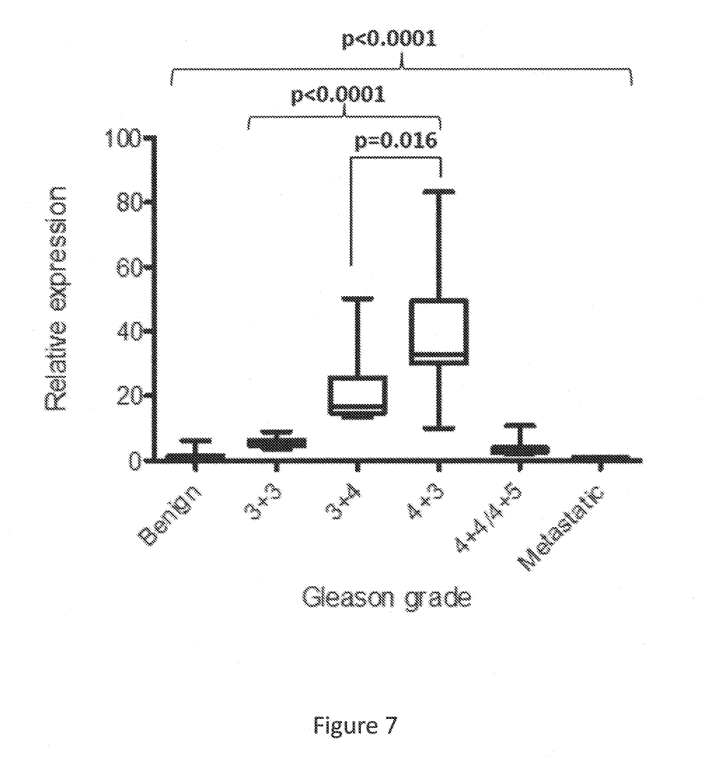

Circulating mRNA extracted from whole blood and qPCR performed to detect circulating VPS13A mRNA (FIG. 7). There was a highly significant difference between the amount of circulating VPS13A mRNA across all groups (p<0.0001). Levels were raised in lower grade tumours compared to benign but in Gleason 4+4/4+5 and the metastatic group they dipped dramatically. The detectable VPS13A in the metastatic group may reflect the heterogeneous hormone status of this group. Of greatest significance is the significant rise in circulating VPS13A mRNA upon the emergence of Gleason 4 disease. Comparison of Gleason 3+3 with Gleason 3+4 and 4+3 showed a highly significant difference (p<0.0001). Furthermore, there was a significant difference between the VPS13A mRNA in Gleason 3+4 and 4+3 disease (p=0.016) suggesting circulating VPS13A may be able to diagnose aggressive disease.

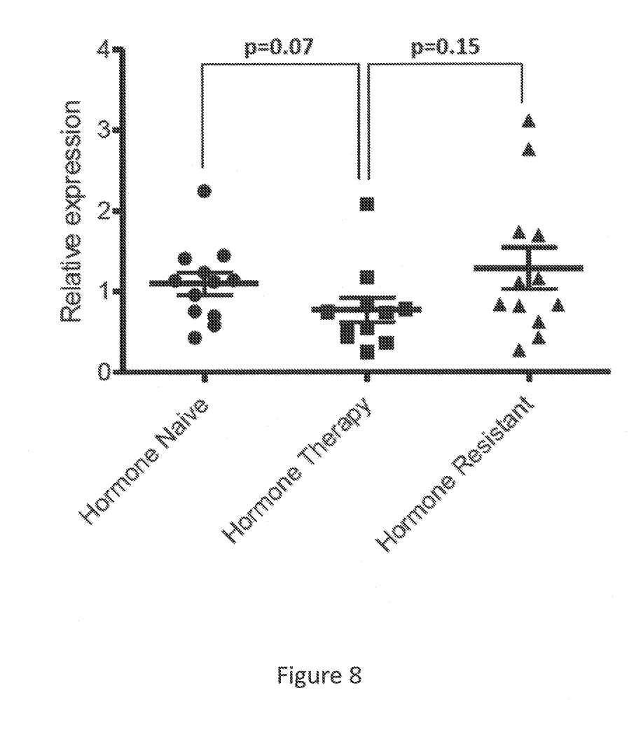

In the first experiment, the metastatic cohort was a mixture of patients who were hormone naïve, on hormone therapy (hormone responsive) and hormone refractory i.e. no longer responding to hormone therapy. To examine this metastatic group more closely, a second experiment examined the expression of circulating RNA in 12 hormone naïve, 12 hormone relapsed and 11 hormone responsive patients (FIG. 8). Although there was a difference between the hormone naïve and hormone responsive patients none of the results were statistically significant.

VPS13A is Associated with Lysosomes and PSA Secretion

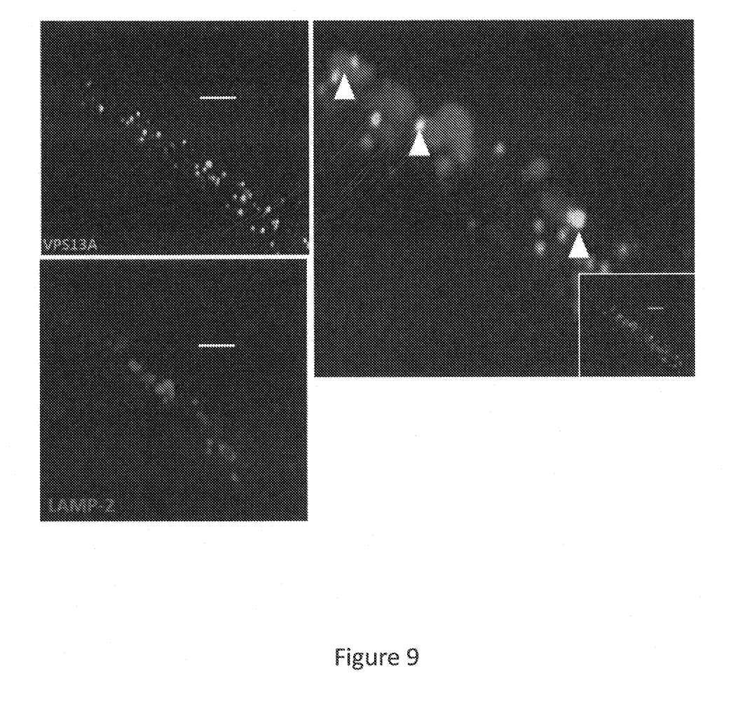

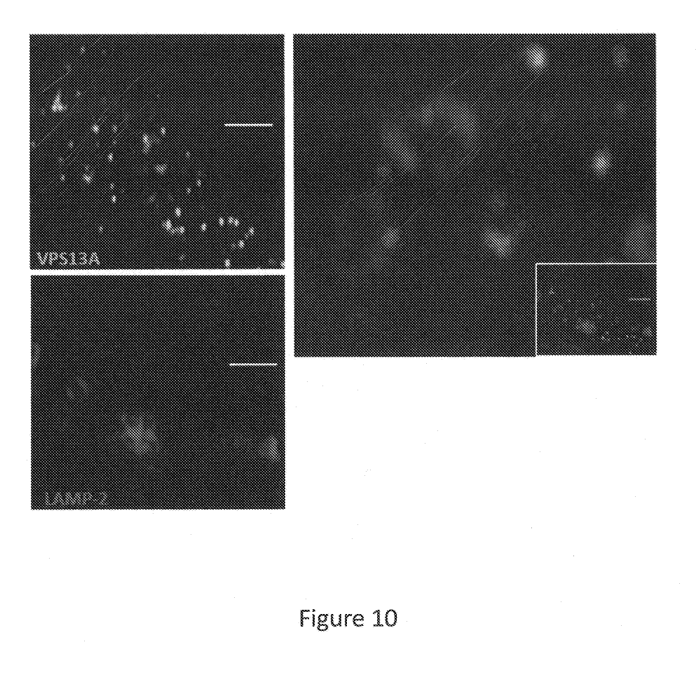

Using confocal microscopy, we have established that VPS13A vesicles do not co-localise with any of the well characterised vesicular compartments such as early/late endosomes or phagosomes. However, we do see an association with lysosomes using the lysosomal marker LAMP2. Control cells show a clear association between VPS13A vesicles and the lysosomal compartment consistent with the ‘kiss and run’ hypothesis of vesicle fusion (FIG. 9). Furthermore, when cells are treated with bafilomycin (which is known to block lysosome fusion with autophagosomes), VPS13A vesicles are seen integrated into the lysosome membrane (FIG. 10). No evidence has been seen for dispersal of the VPS13A throughout the lysosome membrane.

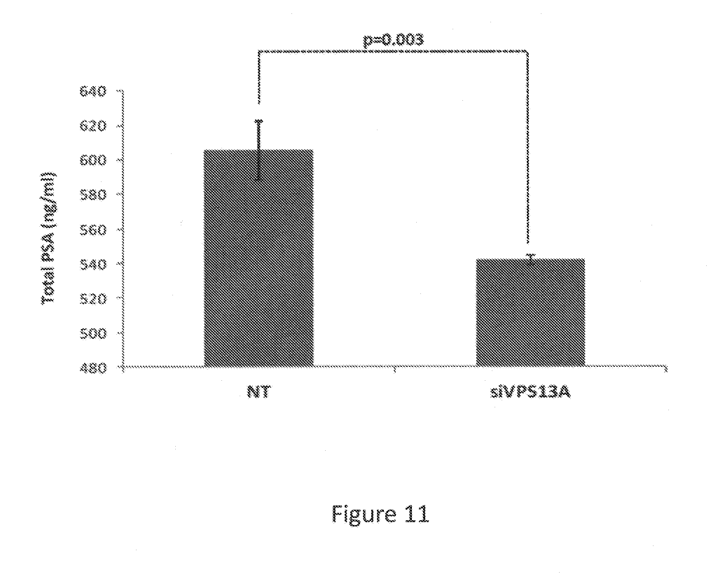

As PSA is known to be processed through lysosomes to give rise to its secreted, active form, we assayed secreted PSA from the media of LNCaP cells stably transfected with siVPS13A or a scrambled control. When VPS13A was knocked down to 30-40% of endogenous levels, the level of PSA detectable in the media decreased significantly (p=0.003) (FIG. 11). This suggests that VPS13A or cargo contained within VPS13A vesicles is altering PSA processing or secretion. These results are consistent with raised PSA and VPS13A in tumour compared to benign tissue.

### VPS13A and Calcium

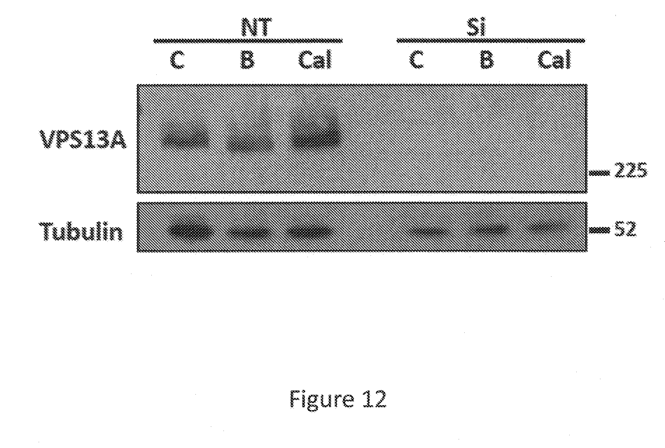

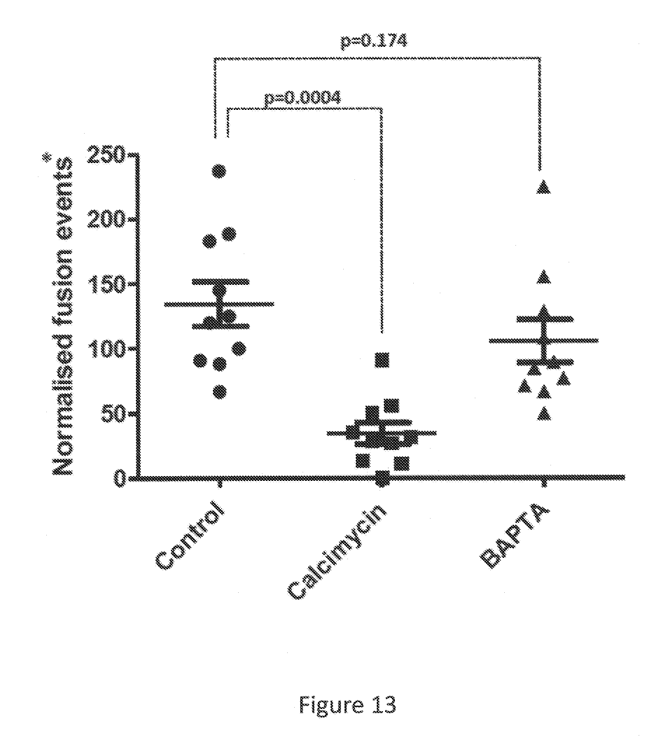

Calcium signalling is known regulate some vesicle fusion events and we have tested this by treating cells with the calcium chelator, BAPTA and the calcium ionophore, calcimycin. When cells were treated with calcimycin, there was an increase in the intracellular levels of VPS13A (FIG. 12). There was no significant change in VPS13A expression following treatment with BAPTA. To determine if this had any impact on the localisation of VPS13A, cells were counterstained with LAMP2 and VPS13A and the number of membrane integration events counted. In cells treated with calcimycin, there was a highly significant decrease in the number of VPS13A vesicles that associated with lysosomes (p=0.0004, FIG. 13). The link between calcium signalling and VPS13A may explain the phenotype of patients with chorea acanthocytosis who suffer from seizures, possibly as a result of defective calcium signalling.

### VPS28

### Materials and Methods

All TMAs were the same as those used for VPS13A and were scored in an identical manner.

### Results

### Qualification of VPS28 as a Diagnostic Marker

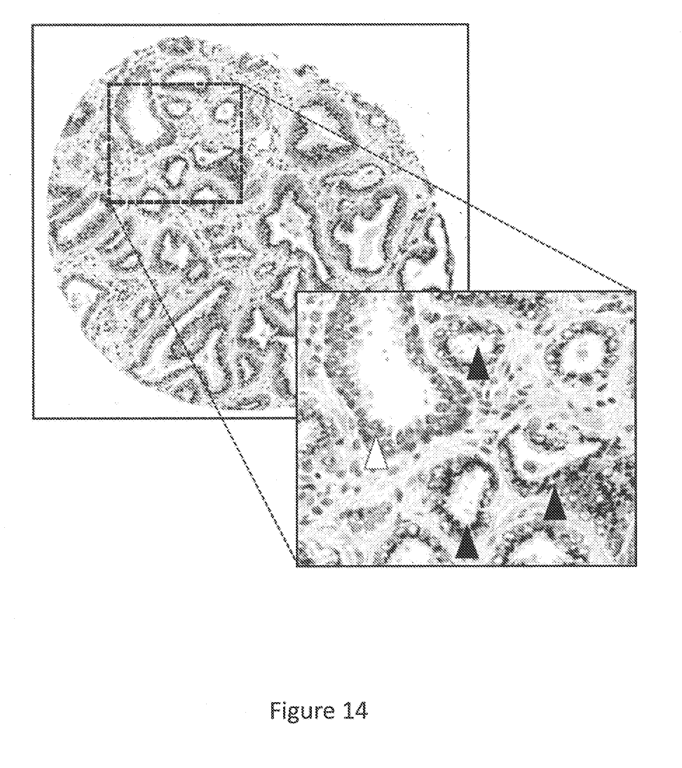

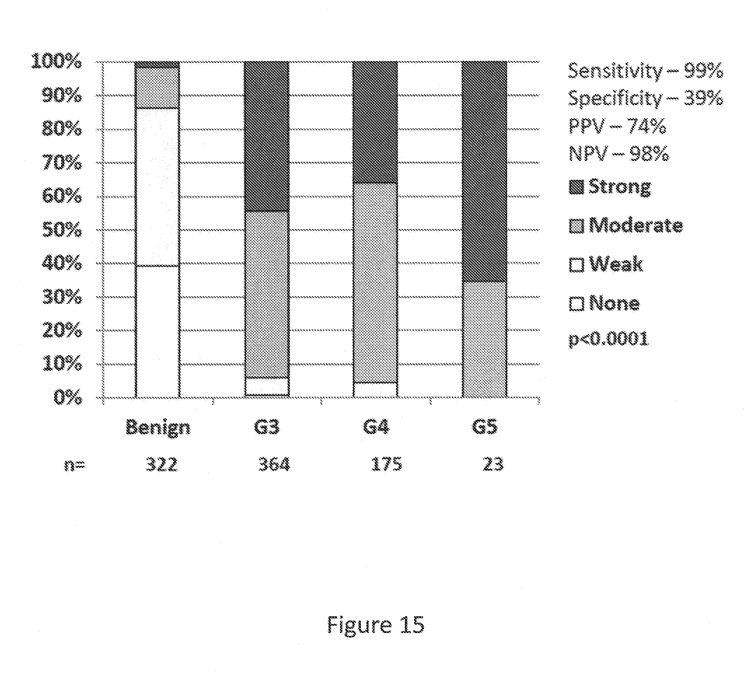

We assessed VPS28 expression using immunohistochemistry (IHC) on prostate tissue. Staining was vesicular and located in the perinuclear region of luminal epithelial cells, consistent with the golgi apparatus (FIG. 14). To assess how specific VPS28 may be for prostate tissue, we used a multi-tumour/normal tissue microarray (TMA) comprised of multiple tissue cores from 16 different organs. VPS28 showed some expression in normal colon but expression in all other tissues was either low or undetectable. The expression of VPS28 was determined in the Cambridge TMA VPS28 was highly significantly up-regulated in tumours when compared to benign (FIG. 15) (p<0001). The positive predictive value (PPV) was 74% and the negative predictive value (NPV) 98%.

### Validation of VPS28 as a Diagnostic Marker

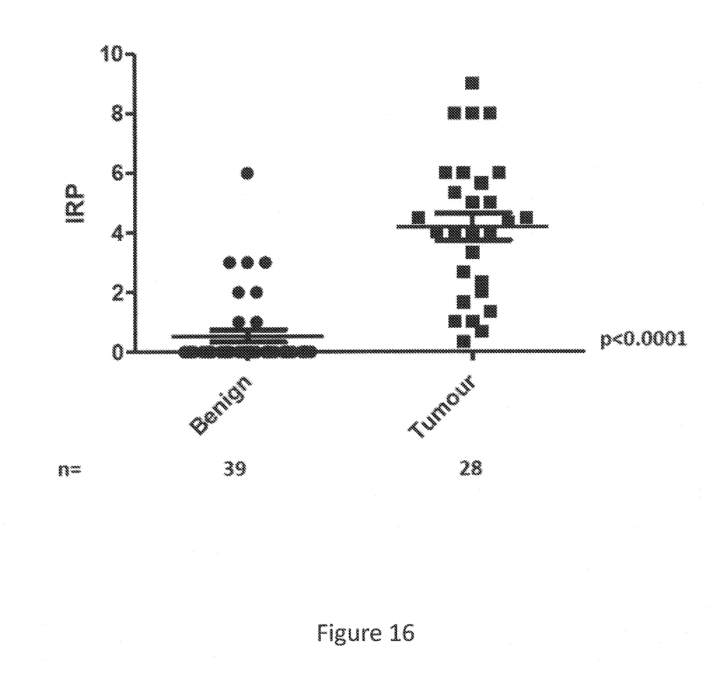

The Karolinska TMA has a limited number of patients with benign as well as tumour cores represented on the TMA. These patients alone were analysed as an additional validation cohort and supported our finding that VPS28 protein expression was significantly different between benign and tumour groups (p<0.0001) (FIG. 16).

### Qualification of VPS28 as a Predictive Marker

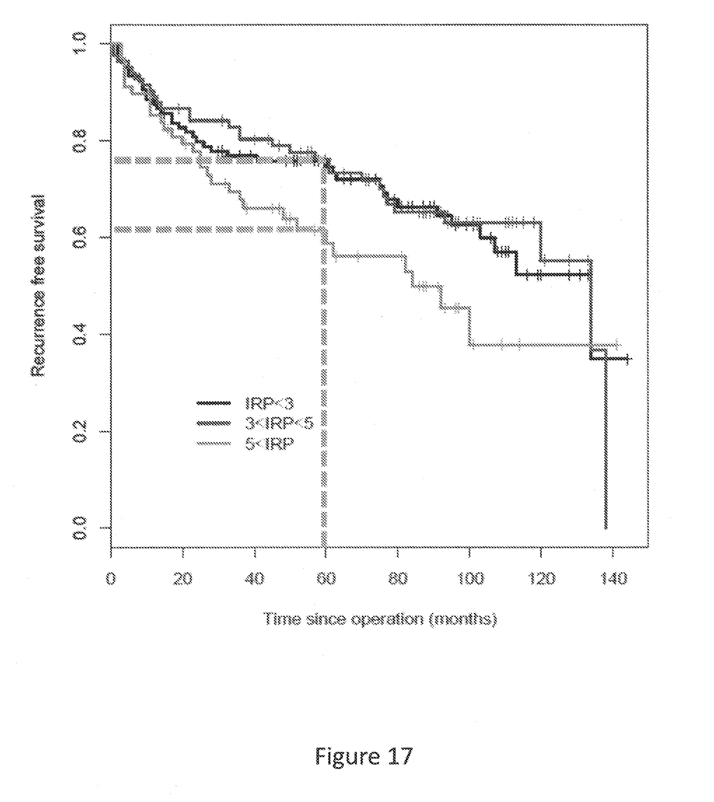

The Karolinska TMA allows analysis of VPS28 as a marker to predict relapse and subsequent death following radical prostatectomy. Taking the immunoreactivity product (IRP) of weak (<3), moderate (>3<5) and strong (>5) VPS28 IHC, Kaplan-Meier curves were generated (FIG. 17). Patients with weak or moderate VPS28 expression had a 25% chance of dying from prostate cancer within 5 years whereas patients with high expression had a 38% chance of relapse and death within 5 years. The hazard ratios show that men with a raised VPS28 (IRP>5) were 1.9 times as likely to relapse and die following radical prostatectomy when the hazard ratio is adjusted for age, Gleason score, extraprostatic extension, positive surgical margin, vesicle invasion, clinical stage, and preoperative PSA (Table 6). However the p-value is not significant.

Circulating VPS13A mRNA as an Alternative Endpoint

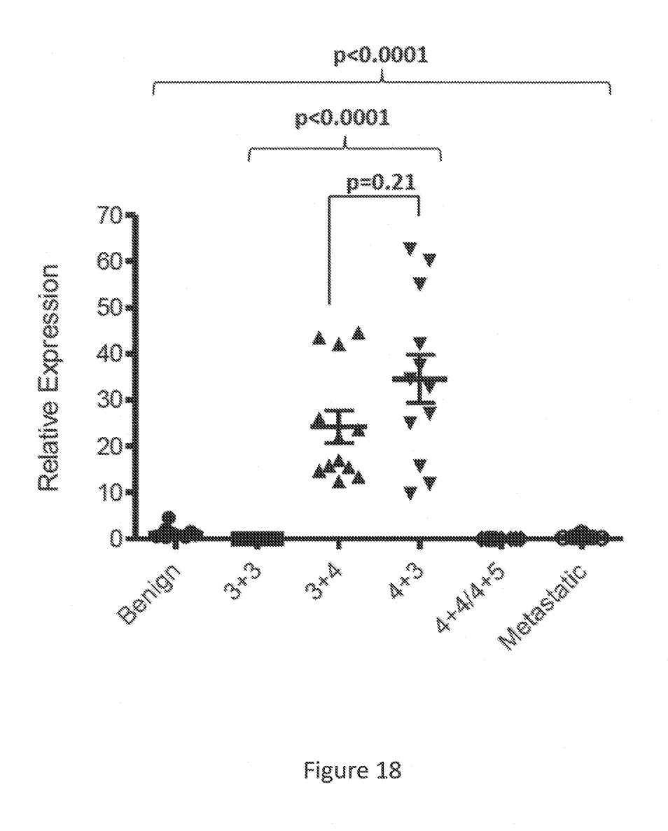

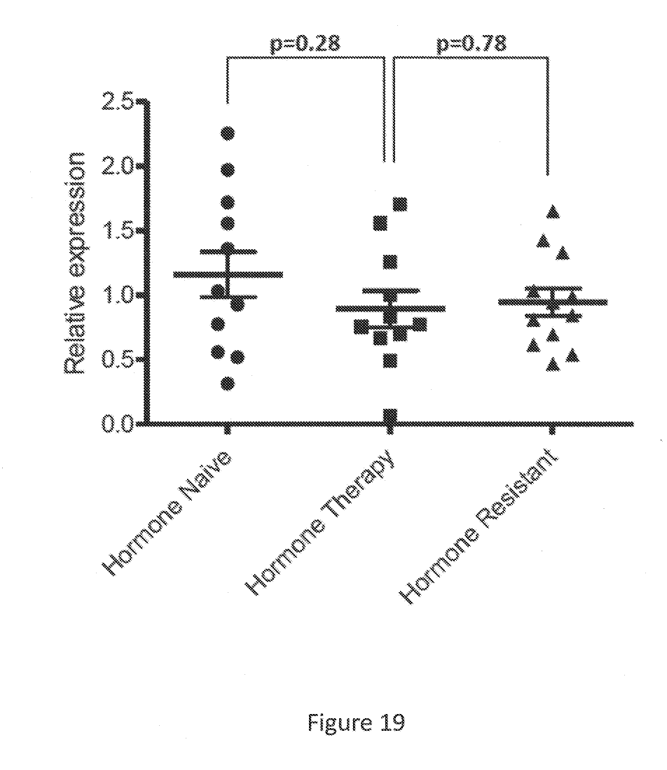

Circulating mRNA extracted from whole blood and qPCR performed to detect circulating VPS28 mRNA (FIG. 18). There was a highly significant difference between the amount of circulating VPS28 mRNA across all groups (p<0.0001). Levels were raised in lower grade tumours compared to benign but in Gleason 4+4/4+5 and the metastatic group they dipped dramatically. The detectable VPS28 in the metastatic group may reflect the heterogeneous hormone status of this group. Of greatest significance is the significant rise in circulating VPS28 mRNA upon the emergence of Gleason 4 disease. Comparison of Gleason 3+3 with Gleason 3+4 and 4+3 showed a highly significant difference (p<0.0001) suggesting circulating VPS28 may be useful to monitor Gleason 6 patients for progression. However, there was no significant difference between the VPS28 mRNA in Gleason 3+4 and 4+3 disease (p=0.21). When we assayed the defined metastatic group (12 hormone naïve, 12 hormone relapsed and 11 hormone responsive patients) there was no significant difference between the groups (FIG. 19).

### NAALADL2

### Materials and Methods

All TMAs were the same as those used for VPS13A and were scored in an identical manner.

### Colony Formation Assay

The CytoSelect™ 96-Well Cell Transformation Assay (Soft Agar Colony Formation) (Cell Biolabs) was used. Briefly, a total of 1250 cells are inoculated per well for each stable cell line in 4 biological replicates. Following an incubation period of 7 days at 37° C. at 5% CO₂, quantification of anchorage independent growth is then determined as per manufacturer's instructions.

### Results

### Qualification and Validation of NAALADL2 as a Diagnostic Marker

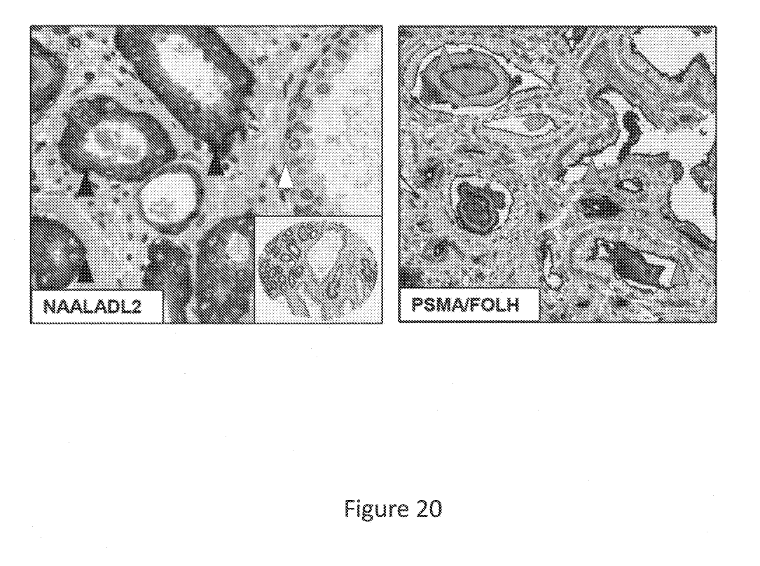

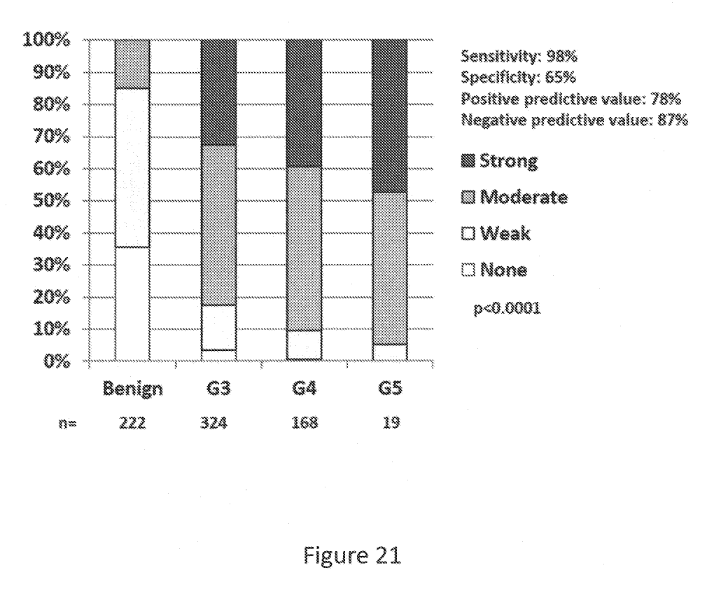

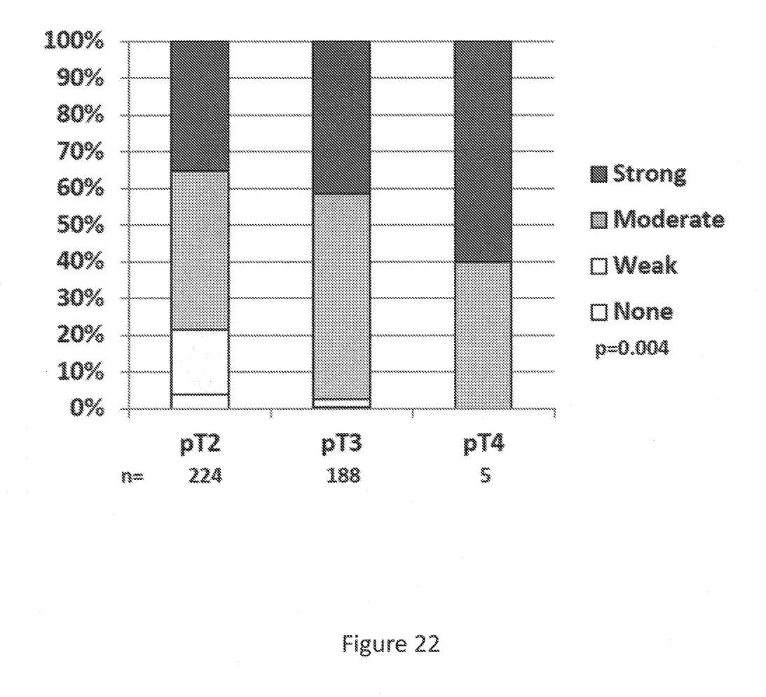

We assessed NAALADL2 expression using immunohistochemistry (IHC) on prostate tissue. Staining was membranous and restricted to the basal cell membrane in sharp contrast to the apical staining seen with PSMA (FIG. 20). To assess how specific NAALADL2 may be for prostate tissue we used a multi-tumour/normal tissue microarray (TMA) comprised of multiple tissue cores from 16 different organs. NAALADL2 showed some expression in breast, pancreas and colon tumours but expression in all other tissues was either low or undetectable. The expression of NAALADL2 was determined in the Cambridge TMA and was highly significantly up-regulated in tumours when stratified by Gleason grade and compared to benign (FIG. 21) (p<0.0001). The positive predictive value (PPV) was 78% and the negative predictive value (NPV) 87%. There was also a significant difference when results were stratified by pathological stage (p=0.004). There was a particularly notable increase in NAALADL2 when the tumour had escaped the prostatic capsule (pT3) (FIG. 22).

### Validation of NAALADL2 as a Diagnostic Marker

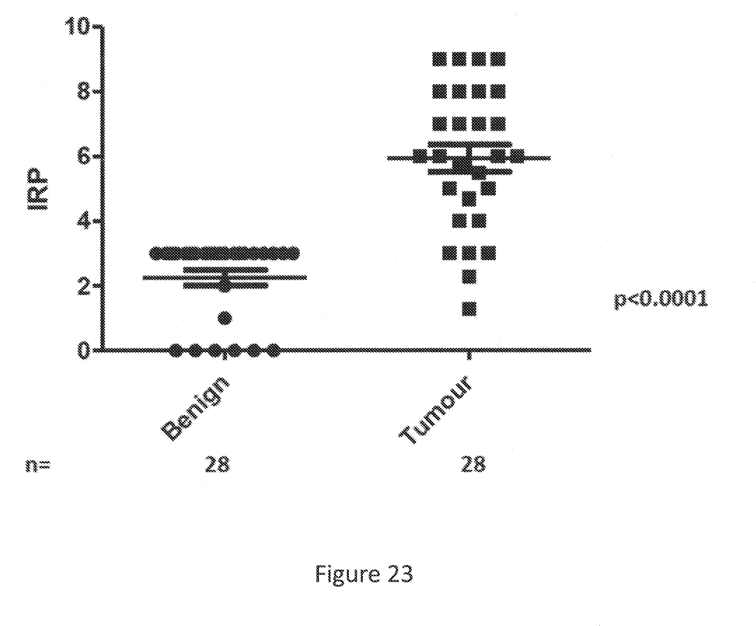

The Karolinska TMA has a limited number of patients with benign as well as tumour cores represented on the TMA. These patients alone were analysed as an additional validation cohort and supported our finding that NAALADL2 protein expression was significantly different between benign and tumour groups (p<0.0001) (FIG. 23).

### Qualification and Validation of NAALADL2 as a Predictive Marker

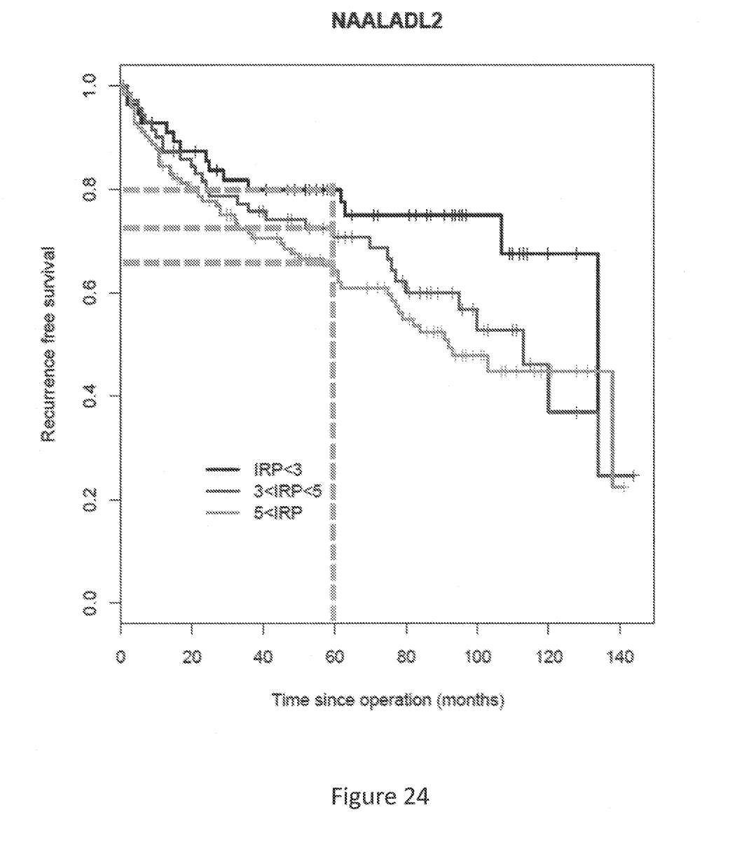

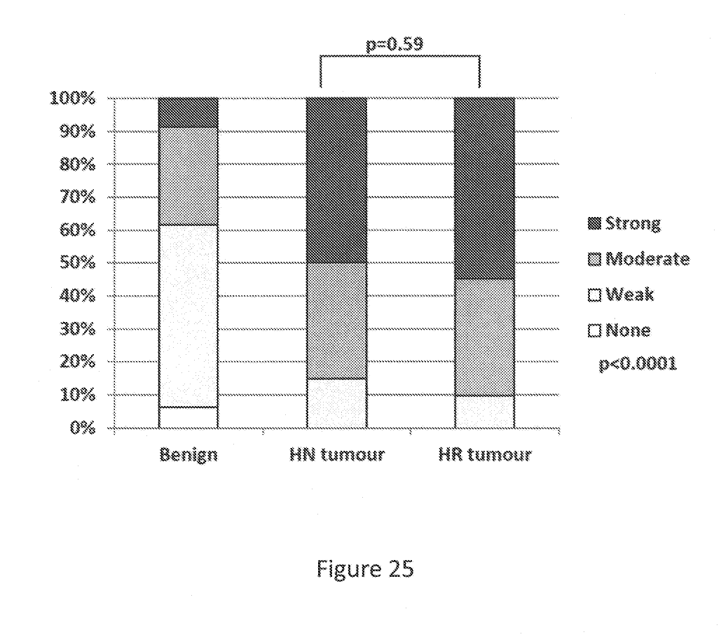

The Karolinska TMA allows analysis of NAALADL2 as a marker to predict relapse and subsequent death following radical prostatectomy. Taking the immunoreactivity product (IRP) of weak (<3), moderate (>3<5) and strong (>5) NAALADL2 IHC, Kaplan-Meier curves were generated (FIG. 24). Patients with weak or no NAALADL2 expression had a 20% chance of dying from prostate cancer within 5 years whereas patients with moderate and high expression had a 27% and 34% chance of relapse and death within 5 years. The hazard ratios show that men with a raised NAALADL2 (IRP>5) were 1.7 times as likely to relapse and die following radical prostatectomy even when the hazard ratio is adjusted for age, Gleason score, extraprostatic extension, positive surgical margin, vesicle invasion, clinical stage, and preoperative PSA (Table 7) (p=0.036). There was no significant difference in NAALADL2 staining between the hormone naïve and hormone refractory tissue on the HR TMA (FIG. 25).

Circulating NAALADL2 mRNA as an Alternative Endpoint

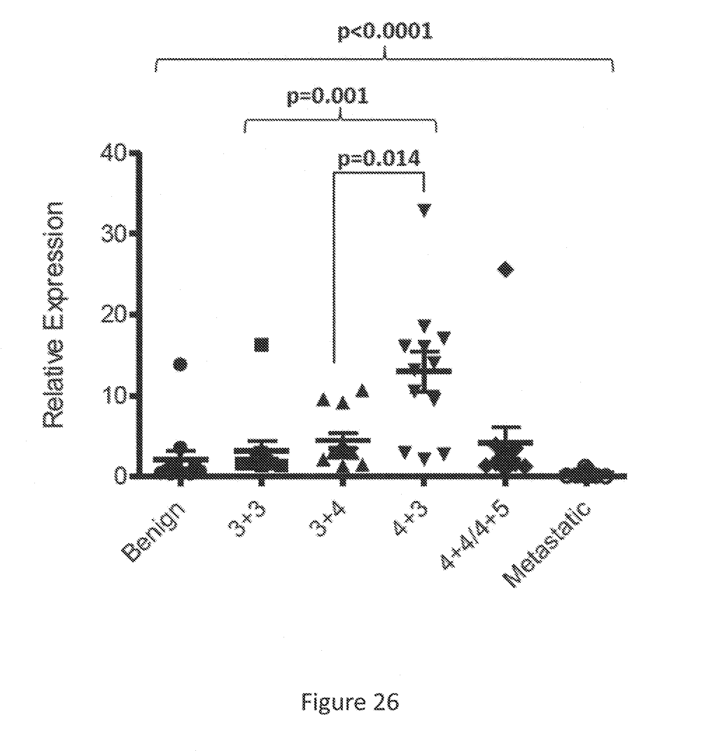

Circulating mRNA extracted from whole blood and qPCR performed to detect circulating NAALADL2 mRNA (FIG. 26). There was a highly significant difference between the amount of circulating NAALADL2 mRNA across all groups (p<0.0001). Levels were raised in lower grade tumours compared to benign but in Gleason 4+4/4+5 and the metastatic group they dipped dramatically. The detectable NAALADL2 in the metastatic group may reflect the heterogeneous hormone status of this group. Of greatest significance is the significant rise in circulating NAALADL2 mRNA upon the emergence of Gleason 4+3 disease. Comparison of Gleason 3+3 with Gleason 3+4 and 4+3 showed a highly significant difference (p=0.001). Furthermore, there was a significant difference between the VPS13A mRNA in Gleason 3+4 and 4+3 disease (p=0.014) suggesting circulating VPS13A may be able to differentiate Gleason 4 disease which requires more urgent treatment than Gleason 3 disease.

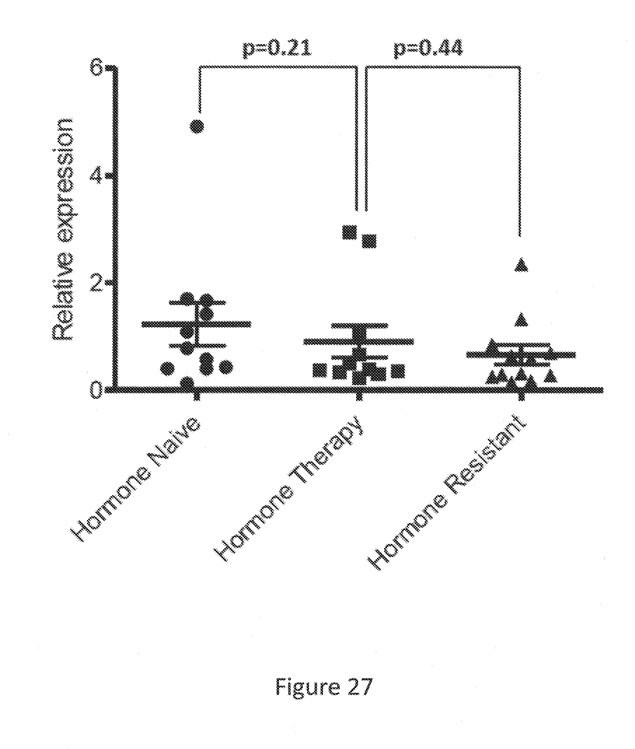

In the first experiment, the metastatic cohort was a mixture of patients who were hormone naïve, on hormone therapy (hormone responsive) and hormone refractory, i.e. no longer responding to hormone therapy. To examine this metastatic group more closely, a second experiment examined the expression of circulating RNA in 12 hormone naïve, 12 hormone relapsed and 11 hormone responsive patients (FIG. 27). There was no significant difference between any of the groups.

### Combined Analysis of VPS13A, VPS28 and NAALADL2

Data from all three markers was analysed to determine if the combination of one or more markers would improve the predictive ability of these markers (Table 8). Although the hazard ratios improved with the addition of come markers, the confidence intervals also increased indicating that for VPS13A and NAALADL2, the addition of the other markers did not increase the predictive power. However, VPS28 was improved by the addition of VPS13A and NAALADL2.

### Study Design

All PAXgene samples were taken from patients enrolled in the ProMPT trial. The study was approved by the institution's ethics committee and informed consent was obtained from all patients. We obtained PAXgene samples from 23 patients—12 control patients who had an elevated PSA and negative biopsy (control cohort), and 11 patients with metastatic prostate cancer. These patients had core biopsy histopathology specimens and TURP chips available for immunohistochemistry. A further 84 PAXgene samples were later obtained for investigation of the expression profile identified in the initial 23 PAXgene samples. The 84 samples consisted of 48 patients with various grades of localized prostate cancer (12 samples of Gleason 3+3, 3+4, 4+3 and 4+4/5 respectively) and 36 patients of different hormone sensitivity categories (12 samples of hormone naïve, hormone therapy and hormone refractory metastatic prostate cancer patients). A 104 patient TMA was constructed from the radical prostatectomy specimens of 104 patients who underwent surgery at Addenbrookes's Hospital, Cambridge for analysis of tissue expression in localized prostate cancer.

### RNA Expression Array

Gene expression analysis was carried out on Illumina Human HT12 version 4 arrays. All data analyses were carried out on R using Bioconductor packages. Raw intensity data from the array scanner was processed using the BASH and HULK algorithms as implemented in the beadarray package. Log 2 transformation and quantile normalisation of the data was performed across all sample groups. Differential expression analysis was carried out using the limma package. Differentially expressed genes were selected using a p-value cut-off of p<0.05 after application of FDR correction for multiple testing applied globally to correct for multiple contrasts.

RNA Extraction and cDNA Formation

RNA was extracted from 2.5 mL of whole blood stored in PAXgene tubes Tepnel using the PAXgene RNA Blood kit (Qiagen Cat no. 762714). RNA was eluted in 80 μL of Elution Buffer. RNA quantification was performed by Absorbance (OD A260 nm) on the Nanodrop ND1000 instrument (Thermo Scientific). Samples with sufficient RNA were normalised to 40 ng/μL in a final volume of 25 μL. 500 ng of RNA was reverse transcribed to cDNA using High Capacity RNA-tocDNA Master Mix (Applied Biosystems) for each sample.

### Real-Time Polymerase Chain Reaction (RT-PCR)

qPCR was performed using the Applied Biosystems 7900HT Real-Time PCR system. qPCR was performed using Sigma primers and SYBR Green. Primers were designed and initially tested by performing RT-PCR on cDNA from cell lines to ensure viability before being used on the PAXgene samples. For each PCR reaction, 1 μL (5 ng) of cDNA and 9 μL of a mastermix containing 5 μL Fast SYBRTM Green, 3.96 μL water and 0.02 μL of forward and reverse primer were added to each well. Assays were performed in triplicate. Relative expression was calculated using the delta-delta Ct method after normalization of Ct values to a housekeeper gene (RPLP2). Primer sequences are given in the supplementary table. Mann-Whitney and one-way ANOVA tests were performed for each gene to determine if differences in expression between groups were significant.

### Immunohistochemistry

All immunohistochemistry was performed using a Bondmax Autostainer using 1.5M EDTA, pH 8.0 for antigen recovery and relevant antibody diluted in a buffer containing 300 mM Tris buffered saline, 1% donkey serum (Sigma Aldrich) and 0.05% Tween. Nuclei were counterstained with haematoxylin and slides coverslipped using DPX. Cambridge TMA—Confirmation of tissue status (Gleason grades and BPH) was conducted by an uropathologist, who assessed and marked the blocks appropriately. Duplicate 0.6-mm tissue cores were cut and constructed according to predetermined tissue microarray (TMA) layout. Multiple 5-Am sections were cut from TMA for Immunohistochemistry. Scoring was done independently by two observers (one an independent specialist urooncology pathologist) who were both blinded to the TMA plan. Staining was classified into the following categories: none, weak, moderate and high, based on intensity. A consensus agreement was reached on intensity and localization on each core. Statistical analysis on immunohistochemistry data was done on the consensus score using GraphPad Prism to perform a one-way ANOVA with Kruskal-Wallis correction test.

TABLE 1
Primers used for the qPCR of the PAXgene target
genes and the control genes (UBC, GAPDH and RPLP2)
(SEQ ID NOs: 1-12).

| | Direction of | |
|---|---|---|
| Gene | Primer | Sequence (5′-3′) |
| UBC | Forward | ACGCACCCTGTCTGACTACAACAT |
| | Reverse | AGGGATGCCTTCCTTGTCTTGGAT |
| RPLP2 | Forward | AAGAAGATCTTGGACAGCGTGGGT |
| | Reverse | TACCCTGGGCAATGACGTCTTCAA |
| GAPDH | Forward | GAAGGTGAAGGTCGGAGTC |
| | Reverse | AAGATGGTGATGGGATTTC |
| VPS13A | Forward | TAGTTGGTGGAGCTGTTGGTGGAT |
| | Reverse | TTTCCTCCACGAGTCATGGCTTCT |
| VPS28 | Forward | GCCTAGAGGATGTTTCATGGG |
| | Reverse | TGTCGTACTTCTCCCTCTCC |
| NAALADL2 | Forward | TGACTCTGAGCAGCAGTGGTCAAT |
| | Reverse | AGCCTTGAGAGTTCCTTTGGCAGA |

TABLE 2
Breakdown of VPS13A staining in the TAPG TMA for diagnosis
with groups of Gleason score <7, =7 and <7 respectively
inside the brackets.

| MI | Benign | | Cancer | | PIN | |
|---|---|---|---|---|---|---|
| 0 | 433 | (248, 96, 89) | 67 | (29, 21, 18) | 0 | (0, 0, 0) |
| 1 | 530 | (319, 118, 93) | 229 | (119, 54, 56) | 0 | (0, 0, 0) |
| 2 | 122 | (73, 28, 21) | 1176 | (493, 358, 325) | 9 | (4, 3, 2) |
| 3 | 2 | (0, 0, 2) | 354 | (99, 127 128) | 1 | (1, 0, 0) |

Total = 2923 (1384, 805, 734)
p < 0.0001 for all groupings.

TABLE 3
Breakdown of VPS13A staining in the TAPG
TMA for diagnosis with Gleason grade.

| | Manual Intensity | | | | |
|---|---|---|---|---|---|
| | 0 | 1 | 2 | 3 | |
| Variable | (N = 13) | (N = 58) | (N = 443) | (N = 195) | p-val |
| Gleason | | | | | |
| score | | | | | |
| <=6 | 9 (2.6%) | 40 (11.8%) | 228 (65.8%) | 67 (19.8%) | 0.000 |
| =7 | 2 (1.1%) | 10 (5.2%) | 111 (58.1%) | 68 (35.6%) | |
| >=8 | 2 (1.1%) | 8 (4.5% | 109 (60.9%) | 60 (33.5%) | |

TABLE 4
VPS13A staining in the Karolinska TMA.

| | | No. | | | | |
|---|---|---|---|---|---|---|
| IRP | No. | recurrence | HR (95% CI) | | HR¹ (95% CI) | |
| <3 | 90 | 24 (26.7) | 1.0 | (ref) | 1.0 | (ref) |
| 3-5 | 90 | 42 (46.7) | 2.0 | (1.2, 3.2) | 1.9 | (1.1, 3.2) |
| >5 | 77 | 37 (48.1) | 2.2 | (1.3, 3.8) | 1.9 | (1.1, 3.3) |

| | | |
|---|---|---|
| Trend test | 0.010 | 0.061 |

¹Hazard ratio adjusted for age, Gleason score, extraprostatic extension, positive surgical margin, vesicle invasion, clinical stage, and preoperative PSA.

TABLE 5
Univariate models for VPS13A manual intensity (MI) in two
categories (0 and 1 versus 2 and 3 or MI of 1 versus 2, 3).

| | Prostate Cancer Survival - MI in two categories | | | |
|---|---|---|---|---|
| | 0, 1 versus 2, 3 (709) | | 1 versus 2, 3 (696) | |
| | | Hazard Ratio | | Hazard Ratio |
| Variable | chi-sq (p-val) | (95% CI) | chi-sq (1.d.f) | (95% CI) |
| Manual Intensit | 2.60 (0.107) | | 2.65 (0.103) | |
| group 1 | | 1.00 | | 1 |
| group 2 | | 1.53 (0.88, 2.63) | | 1.60 (0.87, 2.95) |

TABLE 6
VPS28 staining in the Karolinska TMA.

| | | No. | | | | |
|---|---|---|---|---|---|---|
| | | recurrence | | | | |
| IRP | No. | (%) | HR (95% CI) | | HR¹ (95% CI) | |
| <3 | 105 | 38 (36.2) | 1.0 | (ref) | 1.0 | (ref) |
| 3-5 | 84 | 30 (35.7) | 1.0 | (0.6, 1.5) | 1.1 | (0.7, 1.9) |
| >5 | 68 | 30 (44.1) | 1.5 | (1.0, 2.5) | 1.9 | (1.1, 3.2) |

| | | |
|---|---|---|
| Trend test | 0.299 | 0.175 |

¹Hazard ratio adjusted for age, Gleason score, extraprostatic extension, positive surgical margin, vesicle invasion, clinical stage, and preoperative PSA.

TABLE 7
NAALADL2 staining in the Karolinska TMA.

| | | No. | | | | |
|---|---|---|---|---|---|---|
| IRP | No. | recurrence | HR (95% CI) | | HR¹ (95% CI) | |
| <3 | 56 | 15 (26.8) | 1.0 | (ref) | 1.0 | (ref) |
| 3-5 | 72 | 30 (41.7) | 1.6 | (0.9, 3.0) | 1.5 | (0.8, 2.9) |
| >5 | 124 | 56 (45.2) | 1.9 | (1.1, 3.4) | 1.7 | (0.9, 3.1) |

| | | |
|---|---|---|
| Trend test | 0.0038 | 0.036 |

¹Hazard ratio adjusted for age, Gleason score, extraprostatic extension, positive surgical margin, vesicle invasion, clinical stage, and preoperative PSA.

TABLE 8
The effect of combining VPS13A, VPS28 and NAALADL2.

| | | No | | | | | | |
|---|---|---|---|---|---|---|---|---|
| | | Recurrence | Crude | Adjusted | | | | |
| IRP | No | (%) | HR (95% CI) | HR¹ (95% CI) | HR² (95% CI) | HR³ (95% CI) | HR⁴ (95% CI) | HR⁵ (95% CI) |
| NAALADL2 | | | | | | | | |
| <3 | 52 | 12 (23.1) | 1.0 (ref) | 1.0 (ref) | — | 1.0 (ref) | 1.0 (ref) | 1.0 (ref) |
| 3-5 | 68 | 29 (42.6) | 2.0 (1.0, 3.9) | 1.8 (0.9, 3.7) | — | 1.6 (0.8, 3.3) | 2.0 (1.0, 4.0) | 1.5 (0.7, 3.3) |
| >5 | 116 | 51 (44.0) | 2.2 (1.2, 4.1) | 1.8 (0.9, 3.6) | — | 1.5 (0.7, 3.1) | 2.1 (1.1, 4.0) | 1.3 (0.6, 2.8) |
| VPS13A | | | | | | | | |
| <3 | 86 | 23 (26.7) | 1.0 (ref) | 1.0 (ref) | 1.0 (ref) | — | 1.0 (ref) | 1.0 (ref) |
| 3-5 | 81 | 37 (45.7) | 1.9 (1.1, 3.1) | 1.9 (1.1, 3.3) | 1.6 (0.9, 2.9) | — | 2.0 (1.2, 3.5) | 1.6 (0.9, 3.0) |
| >5 | 69 | 32 (46.4) | 2.1 (1.2, 3.7) | 1.9 (1.1, 3.4) | 1.8 (1.0, 3.6) | — | 2.1 (1.2, 3.8) | 1.5 (0.7, 3.1) |
| VPS28 | | | | | | | | |
| <3 | 88 | 33 (37.5) | 1.0 (ref) | 1.0 (ref) | 1.0 (ref) | 1.0 (ref) | — | 1.0 (ref) |
| 3-5 | 81 | 29 (35.8) | 1.0 (0.6, 1.6) | 1.2 (0.7, 2.1) | 0.8 (0.5, 1.4) | 0.7 (0.5, 1.2) | — | 1.1 (0.6, 1.9) |
| >5 | 67 | 30 (44.8) | 1.6 (1.0, 2.7) | 2.1 (1.2, 3.7) | 1.4 (0.8, 2.4) | 1.2 (0.7, 2.1) | — | 1.9 (1.0, 3.4) |

¹Adjusted tor clinical variables including Gleason score, extraprostatic extension, positive surgical margin, vesicle invasion, clinical stage, preoperative PSA and age at operation,
²adjusted for NAALADL2 immunoreactivity product index,
³adjusted for VPS13A immunoreactivity product index,
⁴adjusted for VPS28 immunoreactivity product index and
⁵adjusted for clinical characteristics and other two biomarkers.

### Sequence Information

The amino acid sequence of human NAALADL2 is shown below (UniProtKB Q58DX5) (SEQ ID NO: 13):

MGENEASLPNTSLQGKKMAYQKVHADQRAPGHSQYLDNDDLQATALDLEW
DMEKELEESGFDQFQLDGAENQNLGHSETIDLNLDSIQPATSPKGRFQRL
QEESDYITHYTRSAPKSNRCNFCHVLKILCTATILFIFGILIGYYVHTNC
PSDAPSSGTVDPQLYQEILKTIQAEDIKKSFRNLVQLYKNEDDMEISKKI
KTQWTSLGLEDVQFVNYSVLLDLPGPSPSTVTLSSSGQCFHPNGQPCSEE
ARKDSSQDLLYSYAAYSAKGTLKAEVIDVSYGMADDLKRIRKIKNVTNQI
ALLKLGKLPLLYKLSSLEKAGFGGVLLYIDPCDLPKTVNPSHDTFMVSLN
PGGDPSTPGYPSVDESFRQSRSNLTSLLVQPISAPLVAKLISSPKARTKN
EACSSLELPNNEIRVVSMQVQTVTKLKTVTNVVGFVMGLTSPDRYIIVGS
HHHTAHSYNGQEWASSTAIITAFIRALMSKVKRGWRPDRTIVFCSWGGTA
FGNIGSYEWGEDFKKVLQKNVVAYISLHSPIRGNSSLYPVASPSLQQLVV
EKNNFNCTRRAQCPETNISSIQIQGDADYFINHLGVPIVQFAYEDIKTLE
GPSFLSEARFSTRATKIEEMDPSFNLHETITKLSGEVILQIANEPVLPFN
ALDIALEVQNNLKGDQPNTHQLLAMALRLRESAELFQSDEMRPANDPKER
APIRIRMLNDILQDMEKSFLVKQAPPGFYRNILYHLDEKTSRFSILIEAW
EHCKPLASNETLQEALSEVLNSINSAQVYFKAGLDVFKSVLDGKN

The coding sequence of human NAALADL2 is shown below (NCBI Gene ID: 254827) (SEQ ID NO: 14):

GGGTCAGTAGAAAGTCAGAAGGTCACAAAGCTTGCAGGGTAAGTGACACA
ACTTGAAACTGCTTGGCCCTCTTTAAAAAGAAATAATAAAATGGGAGAGA
ATGAAGCAAGTTTACCTAACACGTCTTTGCAAGGTAAAAAGATGGCCTAT
CAGAAGGTCCATGCAGATCAAAGAGCTCCAGGACACTCACAGTACTTAGA
CAATGATGACCTTCAAGCCACTGCCCTTGACTTAGAGTGGGACATGGAGA
AGGAACTAGAGGAGTCTGGTTTTGAGCAATTCCAGCTAGACAGTGCTGAG
AATCAGAACCTAGGGCATTCAGAGACTATAGACCTCAATCTTGATTCCAT
TCAACCAGCAACTTCACCCAAAGGAAGGTTCCAGAGACTTCAAGAAGAAT
CTGACTACATTACCCATTATACACGATCTGCACCAAAGAGCAATCGCTGC
AACTTTTGCCACGTCTTAAAAATGCTTTGCACAGCCACCATTTTATTTAT
TTTTGGGATTTTGATAGGTTATTATGTACATACAAATTGCCCTTCAGATG
CTCCATCTTCAGGAACAGTTGATCCTCAGTTATATCAAGAGATTCTCAAG
ACAATCCAGGCAGAAGATATTAAGAAGTCTTTCAGAAATTTGGTACAACT
ATATAAAAATGAAGATGACATGGAAATTTCAAAGAAGATTAAGACTCAGT
GGACCTCTTTGGGCCTAGAAGATGTACAGTTTGTAAATTACTCTGTGCTG
CTTGATCTGCCAGGCCCTTCTCCCAGCACTGTGACTCTGAGCAGCAGTGG
TCAATGCTTTCATCCTAATGGCCAGCCTTGCAGTGAAGAAGCCAGAAAAG
ATAGCAGCCAAGACCTGCTCTATTCATATGCAGCCTATTCTGCCAAAGGA
ACTCTCAAGGCTGAAGTCATCGATGTGAGTTATGGAATGGCAGATGATTT
AAAAAGGATTAGGAAAATAAAAAACGTAACAAATCAGATCGCACTCCTGA
AATTAGGAAAATTGCCACTGCTTTATAAGGTTGGTCCAGTGAATGTTATT
CAGTGGTTTGGTCAATATTTTGCCTTGTTTTGTTGGAATTATATGCTTTT
GTGAGTGTGGAGTGTGTGTGTGCATATAGGTGTGTGAGAGAGAGAAGGGG
AGAGGAAGAAAGAGAGGCAGAGAGTGTCACAGAAAGATGGCTTTTCCACA
TTAGAACATTTTAATTTAAGATATTTAAGAACAATATATTTATGCCCTTA
TTTCTTTAGAGAGAAAATACCTTAAGTCAGGTAACACTGAGTTTGTGGGA
CCTTAATAAAATTGGCATACTCTTCATAATGGTACCTATCTGGAATAGTA
AAAATAGAGAACCACCCTCTGTTCATCTTATGACATATGTGAAACTTCTA
ATCTATTATCAAATGGACTAATTATCATGTTCTCTATGTTAGACAAGTAT
CTAGATGATTTACACCCTTTAGTGATTATTTTGTCAACTATACAACTACA
GTTACTAACTGTGATCAGGATTTTAATTAAAATATAATTGCTAAGAGTAG
CAGAATTTTGATTTATTTTATTTGAATGGAAGTTTATTAACACTCATCCA
CAGATACACTTATGTAATTAAGTTTCTGATGATGAACCAGCACAATAGAC
AGCCACTACTGCTCATTCTCGCTTCATTTCCTTTTTCTATTTAAAAAAAA
AAAAAAAAAAAAAAA

The amino acid sequence of human VPS13A is shown below (UniProt Q96RL7) (SEQ ID NO: 15):

MVFESVVVDVLNRFLGDYVVDLDTSQLSLGIWKGAVALKNLQIKENALSQ
LDVPFKVKVGHIGNLKLIIPWKNLYTQPVEAVLEEIYLLIVPSSRIKYDP
LKEEKQLMEAKQQELKRIEEAKQKVVDQEQHLPEKQDTFAEKLVTQIIKN
LQVKISSIHIRYEDDITNRDKPLSFGISLQNLSMQTTDQYWVPCLHDETE
KLVRKLIRLDNLFAYWNVKSQMFYLSDYDNSLDDLKNGIVNENIVPEGYD
FVFRPISANAKLVMNRRSDFDFSAPKINLEIELHNIAIEFNKPQYFSIME
LLESVDMMAQNLPYRKFKPDVPLHHHAREWWAYAIHGVLEVNVCPRLWMW
SWKHIRKHRQKVKQYKELYKKKLTSKKPPGELLVSLEELEKTLDVFNITI
ARQTAEVEVKKAGYKIYKEGVKDPEDNKGWFSWLWSWSEONTNEQQPDVQ
PETLEEMLTPEEKALLYEAIGYSETAVDPTLLKTFEALKFFVHLKSMSIV
LRENHQKPELVDIVIEEFSTLIVQRPGAQAIKFETKIDSFHITGLPDNSE
KPRLLSSLDDAMSLFQITFEINPLDETVSQRCIIEAEPLEIIYDARTVNS
IVEFFRPPKEVHLAQLTAATLTKLEEFRSKTATGLLYIIETQKVLDLKIN
LKASYIIVPQDGIFSPTSNLLLLDLGHLKVTSKSRSELPDVKQGEANLKE
IMDRAYDSFDIQLTSVQLLYSRVGDNWREARKLSVSTQHILVPMHFNLEL
SKAMVFMDVRMPKFKIYGKLPLISLRISDKKLQGIMELIESIPKPEPVTE
VSAPVKSFQIQTSTSLGTSQISQKIIPLLELPSVSEDDSEEEFFDAPCSP
LEEPLQFPTGVKSIRTRKLQKQDCSVNMTTFKIRFEVPKVLIEFYHLVGD
CELSVVEILVLGLGAEIEIRTYDLKANAFLKEFCLKCPEYLDENKKPVYL
VTTLDNTMEDLLTLEYVKAEKNVPDLKSTYNNVLQLIKVNFSSLDIHLHT
EALLNTINYLHNILPQSEEKSAPVSTTETEDKGDVIKKLALKLSTNEDII
TLQILAELSCLQIFIQDQKCNISEIKIEGLDSEMIMRPSETEINAKLRNI
IVLDSDITAIYKKAVYITGKEVFSFKMVSYMDATAGSAYTDMNVVDIQVN
LIVGCIEVVFVTKFLYSILAFIDNFQAAKQALAEATVQAAGMAATGVKEL
AQRSSRMALDINIKAPVVVIPQSPVSENVFVADFGLITMTNTFHMITESQ
SSPPPVIDLITIKLSEMRLYRSRFINDAYQEVLDLLLPLNLEVVVERNLC
WEWYQEVPCFNVNAQLKPMEFILSQEDITTIFKTLHGNIWYEKDGSASPA
VTKDQYSATSGVTTNASHHSGGATVVTAAVVEVHSRALLVKTTLNISFKT
DDLTMVLYSPGPKQASFTDVRDPSLKLAEFKLENIISTLKMYTDGSTFSS
FSLKNCILDDKRPHVKKATPRMIGLTVGFDKKDMMDIKYRKVRDGCVTDA
VFQEMYICASVEFLQTVANVFLEAYTTGTAVETSVQTWTAKEEVPTQESV
KWEINVIIKNPEIVFVADMTKNDAPALVITTQCEICYKGNLENSTMTAAI
KDLQVRACPFLPVKRKGKITTVLQPCDLFYQTTQKGTDPQVIDMSVKSLT
LKVSPVIINTMITITSALYTTKETIPEETASSTAHLWEKKDTKTLKMWFL
EESNETEKIAPTTELVPKGEMIKMNIDSIFIVLEAGIGHRTVPMLLAKSR
FSGEGKNWSSLINLHCQLELEVHYYNEMFGVWEPLLEPLEIDQTEDFRPW
NLGIKMKKKAKMAIVESDPEEENYKVPEYKTVISFHSKDQLNITLSKCGL
VMLNNLVKAFTEAATGSSADFVKDLAPFMILNSLGLTISVSPSDSFSVLN
IPMAKSYVLKNGESLSMDYIRTKDNDHFNAMTSLSSKLFFILLTPVNHST
ADKIPLTKVGRRLYTVRHRESGVERSIVCQIDTVEGSKKVTIRSPVQIRN
HFSVPLSVYEGDTLLGTASPENEFNIPLGSYRSFIFLKPEDENYQMCEGI
DFEEIIKNDGALLKKKCRSKNPSKESELINIVPEKDNLTSLSVYSEDGWD
LPYIMHLWPPILLRNLLPYKIAYYIEGIENSVFTLSEGHSAQICTAQLGK
ARLHLKLLDYLNHDWKSEYHIKPNQQDISFVSFTCVTEMEKTDLDIAVHM
TYNTGQTVVAFHSPYWMVNKTGRMLQYKADGIHRKHPPNYKKPVLFSFQP
NHFFNNNKVQLMVTDSELSNQFSIDTVGSHGAVKCKGLKMDYQVGVTIDL
SSFNITRIVTFTPFYMIKNKSKYHISVAEEGNDKWLSLDLEQCIPFWPEY
ASSKLLIQVERSEDPPKRIYFNKQENCILLRLDNELGGIIAEVNLAEHST
VITFLDYHDGAATFLLINHTKNELVQYNQSSLSEIEDSLPPGKAVFYTWA
DPVGSRRLKWRCRKSHGEVTQKDDMMMPIDLGEKTIYLVSFFEGLQRIIL
FTEDPRVFKVTYESEKAELAEQEIAVALQDVGISLVNNYTKQEVAYIGIT
SSDVVWETKPKKKARWKPMSVKHTEKLEREFKEYTESSPSEDKVIQLDTN
VPVRLTPTGHNMKILQPHVIALRRNYLPALKVEYNTSAHQSSFRIQIYRI
QIQNQIHGAVFPFVFYPVKPPKSVTMDSAPKPFTDVSIVMRSAGHSQISR
IKYFKVLIQEMDLRLDLGFIYALTDLMTEAEVTENTEVELFHKDIEAFKE
EYKTASLVDQSQVSLYEYFHISPIKLHLSVSLSSGREEAKDSKQNGGLIP
VHSLNLLLKSIGATLTDVQDVVFKLAFFELNYQFHTTSDLQSEVIRHYSK
QAIKQMYVLILGLDVLGNPFGLIREFSEGVEAFFYEPYQGAIQGPEEFVE
GMALGLKALVGGAVGGLAGAASKITGAMAKGVAAMTMDEDYQQKRREAMN
KQPAGFREGITRGGKGLVSGFVSGITGIVTKPIKGAQKGGAAGFFKGVGK
GLVGAVARPTGGIIDMASSTFQGIKRATETSEVESLRPPRFFNEDGVIRP
YRLRDGTGNQMLQVMENGRFAKYKYFTHVMINKTDMLMITRRGVLFVTKG
TFGQLTCEWQYSFDEPTKEPFIVHGRRLRIEAKERVKSVFHAREFGKIIN
FKTPEDARWILTKLQEAREPSPSL

The nucleotide sequence of human VPS1.3A (SEQ ID NO: 16) is shown below. Several splice variants exist and alternate exons are underlined.

ATGGTTTTCGAGTCGGTGGTCGTGGACGTGTTGAACCGGTTCTTGGGGGACTATGTGGTGGACTTGGACA
CGTCCCAGCTCTCTCTGGGCATCTGGAAAGGAGCTGTGGCCCTCAAGAATCTTCAAATTAAAGAAAATGC
CCTGAGTCAACTGGATGTACCATTTAAAGTTAAAGTTGGTCACATAGGTAATCTTAAACTTATAATTCCA
TGGAAAAACCTTTATACTCAACCTGTTGAAGCCGTATTGGAAGAAATTTATTTACTTATAGTGCCTTCTT
CTAGAATAAAATATGATCCTTTAAAAGAAGAGAAACAACTCATGGAAGCAAAGCAACAGGAACTGAAAAG
AATAGAAGAAGCAAAACAAAAAGTAGTTGATCAAGAACAACATCTGCCGGAAAAACAGGACACTTTTGCA
GAAAAATTAGTTACACAGATCATAAAAAATCTTCAGGTGAAAATTTCCAGTATCCATATTCGTTATGAAG
ATGATATCACAAATCGGGACAAACCGCTGTCATTTGGTATTTCCCTTCAAAATCTGAGCATGCAGACAAC
TGATCAATACTGGGTTCCATGTTTACATGATGAAACTGAGAAACTGGTTCGTAAGTTAATCCGATTGGAT
AACCTGTTTGCCTATTGGAATGTGAAGTCTCAGATGTTTTATCTTAGTGATTATGATAACTCCTTGGACG
ACTTGAAGAATGGCATTGTCAATGAAAATATTGTTCCAGAAGGTTATGATTTTGTATTTCGTCCCATATC
TGCTAATGCCAAACTTGTGATGAATCGCCGATCTGATTTTGACTTTTCTGCCCCCAAAATAAACTTGGAA
ATTGAGTTACATAACATAGCAATTGAATTTAATAAACCACAGTATTTCAGTATTATGGAGCTTCTTGAAT
CAGTTGATATGATGGCACAAAATCTGCCATATAGGAAGTTCAAACCTGATGTGCCTCTTCACCACCATGC
CAGAGAATGGTGGGCTTATGCTATACATGGCGTTCTTGAAGTAAATGTTTGCCCCAGGTTATGGATGTGG
TCATGGAAGCATATTAGAAAACATAGGCAAAAAGTGAAGCAATATAAAGAACTGTATAAAAAAAAGTTAA
CAAGTAAGAAGCCACCTGGTGAACTTCTCGTGTCTTTGGAGGAGTTGGAAAAAACCTTGGATGTCTTTAA
TATAACTATAGCTAGACAGACGGCAGAAGTTGAGGTAAAGAAAGCTGGATACAAAATTTACAAAGAAGGA
GTAAAAGATCCAGAGGATAATAAAGGGTGGTTTAGCTGGCTATGGTCTTGGTCAGAACAAAATACTAATG
AACAGCAACCAGATGTTCAACCTGAAACTCTTGAAGAAATGTTGACACCTGAAGAAAAAGCTTTACTCTA
TGAAGCAATTGGCTATAGTGAAACAGCAGTTGATCCAACTTTACTAAAAACATTTGAAGCCTTGAAGTTT
TTTGTCCACTTGAAAAGTATGTCTATTGTTCTAAGAGAAAATCATCAAAAACCTGAGCTGGTAGATATTG
TAATAGAAGAATTTAGCACCTTAATTGTGCAAAGACCAGGAGCACAAGCAATAAAATTTGAAACTAAAAT
AGATTCATTTCATATTACTGGCTTACCAGATAATTCAGAAAAACCCCGCCTCCTGTCTTCATTGGATGAT
GCAATGTCACTTTTCCAAATTACATTTGAGATAAATCCATTAGATGAAACTGTTTCTCAGAGGTGTATCA
TAGAAGCTGAACCTTTAGAAATCATATATGATGCAAGGACAGTGAATAGTATAGTGGAATTCTTCAGACC
TCCAAAAGAGGTACATCTAGCACAGCTCACTGCAGCAACTTTGACAAAACTGGAAGAATTTCGCAGTAAG
ACAGCAACAGGTCTACTGTATATTATTGAAACACAGAAAGTTCTTGATCTCAAAATTAATTTGAAGGCTT
CATATATTATTGTCCCACAAGATGGAATTTTTAGTCCTACATCAAATCTGCTTCTTTTGGACCTTGGTCA
TCTAAAGGTGACGAGTAAAAGTCGTTCTGAATTACCAGATGTGAAACAAGGTGAGGCCAATCTTAAAGAG
ATAATGGATAGAGCTTATGATTCATTTGATATTCAACTTACAAGTGTACAGCTGCTTTACAGTAGAGTTG
GTGATAATTGGAGAGAAGCACGAAAACTCAGTGTATCTACCCAGCATATTTTGGTACCCATGCACTTCAA
TTTGGAACTGTCTAAGGCCATGGTTTTCATGGATGTAAGGATGCCCAAATTCAAGATTTATGGAAAGTTA
CCTCTTATTTCTTTACGAATCTCAGATAAAAAACTACAAGGGATTATGGAATTGATTGAAAGCATTCCAA
AACCTGAACCAGTAACTGAAGTATCTGCCCCTGTCAAATCATTCCAGATTCAAACATCTACTTCTTTGGG
AACATCACAGATTTCACAGAAAATAATTCCTCTCTTGGAACTTCCATCTGTTTCTGAAGATGATTCAGAG
GAGGAATTTTTTGATGCACCATGTAGTCCCTTGGAAGAACCTCTTCAGTTTCCAACTGGAGTTAAAAGTA
TTCGAACCAGAAAGTTACAAAAGCAGGATTGTTCAGTAAATATGACTACATTTAAAATAAGATTTGAAGT
ACCAAAGGTTTTGATCGAGTTTTATCACCTTGITGGAGATTGTGAACTATCTGTGGTAGAAATTCTTGTT
TTAGGATTGGGTGCAGAAATTGAGATTAGAACATACGATTTGAAAGCAAATGCCTTTTTGAAAGAGTTCT
GCTTAAAATGCCCAGAATACTTGGATGAAAACAAGAAACCAGTTTATTTGGTTACAACCCTGGATAACAC
AATGGAAGACCTGTTAACGCTGGAATATGTAAAGGCTGAAAAGAATGTACCCGACTTGAAAAGTACCTAT
AACAATTTTTACAATTGATTAAGGTAAATTTTTCCTCTTTGGATATTCATTTACACACTGAAGCACCTAC
TGAATACAATAAATTATCTTCATAATATCCTTCCGCAATCAGAGGAAAAATCAGCCCCAGTGTCCACTAC
AGAGACTGAAGACAAAGGAGATGTCATTAAAAAATTAGGGCTTGATTCTGAGATGATTATGAGGCCTTCA
GAAACTGAAATAAACGCAAAGCTAAGGAATATAATTGTTTTAGATTCTGATATAACAGCTATATACAAAA
AGGCTGTTTATATCACTGGAAAAGAAGTTTTCAGCTTCAAAATGGTTTCTTACATGGATGCAACTGCTGG
TTCTGCATACACAGATATGAATGTGGTTGACATTCAGGTTAATTTAATAGTTGGTTGCATTGAAGTAGTT
TTTGTCACGAAATTTCTATATTCTATATTGGCTTTTATAGATAATTTTCAGGCAGCTAAACAAGCCTTGG
CTGAGGCAACTGTTCAGGCAGCTGGAATGGCTGCTACTGGTGTAAAAGAACTCGCACAAAGGAGTTCCAG
AATGGCACTGGATATTAACATCAAAGCCCCAGTTGTGGTCATCCCGCAGTCTCCAGTTTCTGAAAATGTT
TTTGTTGCTGATTTTGGACTAATTACAATGACAAATACCTTTCATATGATAACAGAGAGCCAGAGCTCTC
CCCCACCTGTTATTGATTTGATAACAATAAAGCTGAGTGAAATGCGACTATACAGATCTCGATTTATTAA
TGATGCATACCAGGAAGTACTGGATCTACTCCTGCCATTAAATCTTGAGGTTGTGGTTGAACGAAATTTA
TGCTGGGAGTGGTACCAGGAAGTTCCTTGTTTTAATGTAAATGCTCAGCTGAAACCAATGGAGTTCATTC
TTAGTCAAGAAGATATAACAACTATTTTTAAAACATTGCATGGCAATATATGGTATGAAAAAGATGGTAG
TGCCTCACCTGCTGTAACAAAAGACCAATACAGTGCCACTAGTGGAGTTACTACTAATGCTTCACACCAT
TCAGGAGGAGCAACTGTGGTGACAGcTGCTGTGGTAGAAGTACATTCACGTGCCTTACTAGTTAAGACAA
CACTAAACATAAGCTTCAAAACTGATGATCTCACCATGGTGCTGTATAGTCCAGGTCCTAAACAGGCTTC
CTTTACAGATGTTCGTGATCCTTCTCTGAAACTTGCTGAATTTAAATTGGAGAATATTATAAGTACTTTA
AAAATGTATACAGATGGCTCAACATTTTCTTCCTTCTCATTAAAAAACTGTATTTTAGATGATAAAAGAC
CTCATGTCAAGAAAGCAACTCCTCGAATGATAGGACTGACAGTTGGTTTTGACAAAAAAGACATGATGGA
TATAAAGTACAGGAAAGTCAGAGATGGTTGTGTGACTGATGCGGTCTTTCAAGAAATGTATATTTGTGCA
AGCGTAGAATTTCTGCAGACTGTTGCAAATGTCTTTCTTGAGGCCTACACCACAGGCACTGCTGTAGAAA
CCAGTGTGCAAACATGGACTGCTAAGGAAGAAGTACCTACACAGGAATCAGTGAAGTGGGAAATTAATGT
TATTATTAAAAATCCTGAAATTGTGTTTGTAGCTGACATGACAAAAAATGATGCTCCTGCTTTAGTCATT
ACAACACAATGTGAAATTTGCTATAAAGGTAACCTTGAAAATAGTACAATGACTGCTGCCATTAAAGATC
TCCAAGTGAGAGCCTGCCCGTTTCTTCCAGTCAAGAGAAAAGGCAAAATCACTACTGTTTTGCAGCCCTG
TGACTTGTTTTATCAAACTACTCAGAAAGGTACAGATCCACAAGTGATCGATATGTCAGTAAAATCCCTG
ACACTAAAGGTTTCACCAGTTATTATAAATACTATGATTACCATAACTTGAGCACTGTATACAACTAAGG
AAACCATCCCAGAAGAAACGGCTTCTTCTACTGCACATTTATGGGAAAAGAAGGATACAAAGACTTTAAA
AATGTGGTTTCTTGAAGAATCAAATGAAACTGAAAAAATAGCTCCCACAACTGAATTGGTACCCAAAGGC
GAGATGATAAAAATGAACATTGATTCTATTTTTATAGTTCTTGAGGCTGGAATTGGTCATAGAACAGTAC
CTATGCTTCTGGCAAAGTCACGTTTTTCAGGGGAAGGCAAAAACTGGAGTTCCCTAATAAATCTGCACTG
TCAGCTTGAGCTAGAAGTGCATTATTATAATGAAATGTTTGGTGTATGGGAGCCTTTGCTTGAACCCTTA
GAAATTGATCAGACTGAGGATTTTAGACCATGGAATCTTGGTATCAAGATGAAAAAGAAAGCAAAAATGG
CCATTGTTGAGTCAGATCCTGAAGAAGAAAACTACAAAGTGCCAGAATATAAAACTGTCATCAGTTTCCA
TTCAAAAGACCAATTAAACATTACATTATCCAAATGTGGTCTTGTAATGTTAAACAATTTAGTCAAGGCA
TTTACAGAAGCTGCCACTGGATCTTCAGCTGACTTCGTAAAGGATCTAGCACCATTTATGATTTTAAATT
CCCTTGGACTTACTATTTCTGTTTCGCCAAGTGATTCTTTTAGTGTACTCAACATTCCTATGGCAAAATC
ATATGTATTGAAAAATGGAGAAAGTTTAAGTATGGATTATATCCGAACCAAGGACAATGATCATTTCAAT
GCAATGACCAGCCTAAGCAGCAAACTCTTCTTCATTCTTCTTACACCTGTTAACCATTCTACTGCTGATA
AGATTCCTTTAACAAAAGTGGGACGACGTCTGTACACTGTAAGACACAGAGAGTCTGGCGTTGAAAGATC
TATTGTTTGTCAAATTGATACAGTAGAACGAAGTAACAAGGTCACAATTCGCTCCCCAGTGCAGATAAGA
AATCATTTTTCAGTCCCACTGTCTGTTTACGAAGGGGATACCTTATTGGGAACTGCCTCACCTGAAAATG
AATTCAACATACCATTAGGATCTTACCGATCATTCATTTTTCTGAAGCCAGAAGATGAGAACTATCAAAT
GTGTGAAGGAATTGACTTTGAAGAGATTATAAAAAATGATGGTGCTCTTCTAAAGAAGAAATGTAGATCT
AAAAACCCTTCTAAGGAATCATTTCTCATTAATATTGTTCCAGAAAAAGATAATTTAACATCTCTATCAG
TGTATTCAGAAGATGGTTGGGATTTACCATACATAATGCATTTGTGGCCACCTATCCTGCTCCGAAATCT
TCTTCCTTACAAAATTGCTTATTATATAGAGGGAATTGAAAATTCGGTTTTTACTCTAAGTGAAGGACAT
TCAGCCCAGATTTGTACTGCACAGTTGGGTAAAGCCAGGCTACATTTAAAATTACTTGACTATCTCAATC
ACGATTGGAAAAGTGAATATCACATAAAGCCTAATCAGCAAGACATTAGTTTTGTCAGTTTTACTTGTGT
TACAGAAATGGAAAAGACTGATTTAGATATTGCTGTCCATATGACTTACAATACTGGTCAGACAGTTGTG
GCATTTCATAGTCCTTATTGGATGGTCAATAAAACTGGCCGCATGTTACAGTACAAAGCAGACGGAATTC
ATCGAAAGCATCCACCTAATTATAAAAAGCCAGTTCTCTTTTCTTTTCAGCCAAATCACTTTTTTAATAA
CAATAAGGTTCAACTTATGGTAACTGATAGTGAGTTGTCCAATCAGTTTTCAATTGATACTGTTGGTAGT
CATGGAGCTGTTAAATGTAAAGGCCTGAAAATGGACTATCAAGTTGGTGTCACTATAGACCTGAGCAGTT
TTAACATTACTAGAATTGTGACATTTACCCCTTTTTATATGATTAAAAACAAAAGCAAATACCATATATC
AGTGGCTGAAGAAGGAAATGATAAATGGCTCTCTCTTGATTTGGAGCAGTGTATCCCCTTTTGGCCTGAG
TATGCTTCTAGTAAACTTCTTATTCAAGTCGAAAGGAGTGAAGATCCTCCCAAAAGGATATATTTTAACA
AGCAGGAAAATTGTATTCTATTGCGTCTAGATAACGAGCTTGGAGGTATTATAGCAGAAGTGAATTTGGC
CGAGCATTCTACAGTTATTACATTTTTAGATTATCATGATGGAGCAGCTACATTCCTCTTAATAAATCAC
ACAAAGAATGAACTTGTTCAATACAATCAAAGTTCTCTCAGTGAAATAGAAGATTCCCTCCCTCCTGGTA
AAGCCGTGTTTTATACATGGGCTGATCCGGTGGGCTCTAGAAGGCTGAAGTGGAGATGTAGAAAAAGCCA
TGGTGAAGTAACACAGAAGGATGATATGATGATGCCTATAGATTTGGGGGAAAAGACAATATATTTAGTT
TCATTCTTTGAAGGTTTACAACGCATTATTTTATTCACTGAAGATCCAAGGGTATTTAAAGTAACATATG
AAAGTGAGAAAGCAGAGTTAGGAGAGCAAGAAATTGCAGTGGCATTACAAGATGTTGGAATTTCTCTTGT
CAACAATTACACGAAGCAAGAAGTAGCCTATATAGGCATTACAAGTTCTGATGTGGTTTGGGAAACAAAG
CCCAAGAAGAAGGCAAGATGGAAGCCAATGAGTGTAAAGCACACTGAGAAGTTAGAGAGAGAATTTAAGG
AATATACTGAATCTTCTCCTTCAGAAGATAAGGTTATTCAGTTGGACACTAATGTTCCGGTTCGCCTAAC
CCCTACTGGTCATAACATGAAAATTCTGCAGCCGCATGTAATAGCTCTACGAAGAAATTATCTTCCAGCA
TTAAAAGTGGAATATAACACATCTGCACATCAATCATCATTTAGAATTCAGATTTACAGAATACAGATCC
AAAATCAGATACATGGTGCTGTATTTCCCTTTGTGTTTTATCCTGTTAAACCTCCAAAGTCGGTCACCAT
GGATTCAGCACCAAAGCCCTTTACAGATGTCAGTATTGTCATGAGATCTGCAGGACATTCCCAGATATCA
CGTATTAAGTATTTCAAAGTATTGATTCAAGAAATGGATCTCAGGTTAGATCTTGGGTTTATCTATGCTT
TAACAGACCTTATGACAGAAGCTGAGGTGACTGAAAATACAGAGGTTGAGCTTTTTCATAAAGATATAGA
AGCTTTCAAAGAAGAATATAAAACAGCCTCATTAGTAGATCAATCACAAGTCAGCCTCTATGAATATTTT
CATATATCTCCTATCAAGTTACATTTAAGTGTTTCACTGAGTTCCGGCAGAGAAGAAGCTAAAGATTCAA
AACAAAATGGAGGACTGATTCCAGTTCATTCTTTAAATCTTTTGCTGAAGAGTATTGGTGCCACACTGAC
AGATGTACAAGATGTAGTTTTTAAGCTTGCATTTTTTGAACTCAACTATCAGTTCCATACAACATCCGAT
CTACAGTCTGAAGTCATAAGACACTATTCAAAACAGGCCATTAAGCAGATGTATGTACTCATTCTTGGAC
TTGATGTTTTGGGAAATCCATTTGGCTTAATTAGAGAATTTTCTGAAGGTGTAGAAGCATTTTTTTATGA
ACCTTACCAGGGAGCCATCCAGGGTCCTGAAGAGTTTGTGGAAGGAATGGCACTAGGACTTAAGGCACTA
GTTGGTGGAGCTGTTGGTGGATTGGCTGGTGCTGCCTCCAAAATCACCCGTGCTATCGCTAAGGGGGTAG
CAGCTATGACCATGGATGAAGACTACCAACAGAAGAGAAGAGAAGCCATGAATAAGCAACCAGCTGGTTT
TAGAGAAGGCATCACTCGTGGAGGAAAAGGCTTAGTTTCTGGATTTGTTAGTGGCATAACAGGAATTGTT
ACAAAACCAATCAAAGGAGCTCAAAAAGGAGGAGCAGCTGGTTTCTTTAAAGGTGTTGGGAAAGGTTTAG
TAGGAGCGGTAGCAAGGCCAACTGGAGGCATCATAGACATGGCTAGCAGTACATTTCAGGGAATAAAAAG
AGCTACAGAGACTTCTGAAGTGGAGAGTCTGCGACCTCCTCGGTTCTTCAATGAAGATGGAGTTATCAGA
CCGTACAGGTTGAGGGATGGGACTGGAAATCAAATGTTACAGGTCATGGAAAATGGAAGATTTGCAAAAT
ACAAATATTTTACCCATGTCATGATCAATAAGACAGATATGCTAATGATAACCAGACGTGGTGTATTGTT
TGTAACAAAGGGAACATTTGGACAACTCACGTGTGAGTGGCAGTATAGTTTTGATGAATTTACCAAAGAG
CCATTCATTGTTCATGGGAGAAGATTGCGCATTGAAGCAAAGGAACGAGTGAAGTCTGTATTTCATGCCA
GAGAGTTTGGAAAAATAATTAACTTCAAGACCCCAGAGGATGCCAGGTGGATCCTCACAAAGCTACAAGA
AGCAAGAGAACCTTCTCCGAGCCTCTGA

The protein sequence of human VPS13A (SEQ ID NO: 17) is shown again with the amino acids encoded by alternate exons underlined. Amino acids encoded across a splice junction are in bold.

MVFESVVVDVLNRFLGDYVVDLDTSQLSLGIWKGAVALKNLQIKENALSQLDVPFKVKVGHIGNLKLIIP
WKNLYTQPVEAVLEEIYLLIVPSSRIKYDPLKEEKQLMEAKQQELKRIEEAKQKVVDQEQHLPEKQDTFA
EKLVTQIIKNLQVKISSIHIRYEDDITNRDKPLSFGISLQNLSMQTTDQYWVPCLHDETEKLVRKLIRLD
NLFAYWNVESQMFYLSDYDNSLDDLKNGIVNENIVPEGYDFVFRPISANAKLVMNRRSDFDFSAPKINLE
IELHNIAIEFNKPQYFSIMELLESVDMMAQNLPYRKFKPDVPLHHHAREWWAYAIHGVLEVNVCPRLWMW
SWKHIRKHRQKVKQYKELYKKKLTSKKPPGELLVSLEELEKTLDVFNITTARQTAEVEVKKAGYKIYKEG
VKDPEDNKGWFSWLWSWSEQNTNEQQPDVQPETLEEMLTPEEKALLYEAIGYSETAVDPTLLKTFEALKF
FVHLKSMSIVLRENHQKPELVDIVIEEFSTLIVQRPGAQAIKFETKIDSFHITGLPDNSEKPRLLSSLDD
AMSLFQITFEINPLDETVSQRCIIEAEPLEIIYDARTVNSIVEFFRPPKEVHLAQLTAATLTKLEEFRSK
TATGLLYIIETQKVLDLKINLKASYIIVPQDGIFSPTSNLLLLDLGHLKVTSKSRSELPDVKQGEANLKE
IMDRAYDSFDIQLTSVQLLYSRVGDNWREARKLSVSTQHILVPMHFNLELSKAMVFMDVRMPKFKIYGKL
PLISLRISDKKLQGIMELIESIPKPEPVTEVSAPVKSFQIQSTSTLGTSQISQKIIPLLELPSVSEDDSE
EEFFDAPCSPLEEPLQFPTGVKSIRTRKLQKQDCSVNMTTFKIRFEVPKVLIEFYHLVGDCELSVVEILV
LGLGAEIEIRTYDLKANAFLKEFCLKCPEYLDENKKPVYLVTTLDNTMEDLLTLEYVKAEKNVPDLKSTY
NNVLQLIKVNFSSLDIHLHTEALLNTINYLHNILPQSEEKSAPVSTTETEDKGDVIKKLGLDSEMIMRPS
ETEINAKLRNIIVLDSDITAIYKKAVYITGKEVFSFKMVSYMDATAGSAYTDMNVVDIQVNLIVGCIEVV
FVTKFLYSILAFIDNFQAAKQALAEATVQAAGMAATGVKELAQRSSRMALDINIKAPVVVIPQSPVSENV
FVADFGLITMTNTFHMITESQSSPPPVIDLITIKLSEMRLYRSRFINDAYQEVLDLLLPLNLEVVVERNL
CWEWYQEVPCFNVNAQLKPMEFILSQEDITTIFKTLHGNIWYEKDGSASPAVTKDQYSATSGVTTNASHH
SGGATVVTAAVVEVHSRALLVKTTLNISFKTDDLTMVLYSPGPKQASFTDVRDPSLKLAEFKLENIISTL
KMYTDGSTFSSFSLKNCILDDKRPHVKKATPRMIGLTVGFDKKDMMDIKYRKVRDGCVTDAVFQEMYICA
SVEFLQTVANVFLEAYTTGTAVETSVQTWTAKEEVPTQESVKWEINVIIKNPEIVFVADMTKNDAPALVI
TTQCEICYKGNLENSTMTAAIKDLQVRACPFLPVKRKGKITTVLQPCDLFYQTTQKGTDPQVIDMSVKSL
TLKVSPVIINTMITITSALYTTKETIPEETASSTAHLWEKKDTKTLKMWFLEESNETEKIAPTTELVPKG
EMIKMNIDSIFIVLEAGIGHRTVPMILAKSRFSGEGKNWSSLINLHCQLELEVHYYNEMEGVWEPLLEPL
EIDQTEDFRPWNLGIKMKKKAKMAIVESDPEEENYKVPEYKTVISFHSKDQLNITLSKCGLVMLNNLVKA
FTEAATGSSADFVKDLAPFMILNSLGLTISVSPSDSFSVLNIPMAKSYVLKNGESLSMDYIRTKDNDHFN
AMTSLSSKLFFILLTPVNHSTADKIPLTKVGRRLYTVRHRESGVERSIVCQIDTVEGSKKVTIRSPVQIR
NHFSVPLSVYEGDTLLGTASPENEFNIPLGSYRSFIFLKPEDENYQMCEGIDFEEIIKNDGALLKKKCRS
KNPSKESFLINIVPEKDNLTSLSVYSEDGWDLPYIMHLWPPILLRNLLPYKIAYYIEGIENSVFTLSEGH
SAQICTAQLGKARLHLKLLDYLNHDWKSEYHIKPNQQDISFVSFTCVTEMEKTDLDIAVHMTYNTGQTVV
AFHSPYWMVNKTGRMLQYKADGIHRKHPPNYKKPVLFSFQPNHFFNNNKVQLMVTDSELSNQFSIDTVGS
HGAVKCKGLKMDYQVGVTIDLSSFNITRIVTFTPFYMIKNKSKYHISVAEEGNDKWLSLDLEQCIPFWPE
YASSKLLIQVERSEDPPKRIYFNKQENCILLRLDNELGGIIAEVNLAEHSTVITFLDYHDGAATFLLINH
TKNELVQYNQSSLSEIEDSLPPGKAVFYTWADPVGSRRLKWRCRKSHGEVTQKDDMMMPIDLGEKTIYLV
SFFEGLQRIILFTEDPRVFKVTYESEKAELAEQEIAVALQDVGISLVNNYTKQEVAYIGITSSDVVWETK
PKKKARWKPMSVKHTEKLEREFKEYTESSPSEDKVIQLDTNVPVRLTPTGHNMKILQPHVIALRRNYLPA
LKVEYNTSAHQSSFRIQTYRIQIQNQIHGAVFPFVFYPVKPPKAVTMDSAPKPFTDVSIVMRSAGHSQIS
RIKYFKVLIQEMDLRLDLGFIYALTDLMTEAEVTENTEVELFHKDIEAFKEEYKTASLVDQSQVSLYEYF
HISPIKLHLSVSLSSGREEAKDSKQNGGLIPVHSLNLLLKSIGATLTDVQDVVFKLAFFELNYQFHTTSD
LQSEVIRHYSKQAIKQMYVLILGLDVLGNPFGLIREFSEGVEAFFYEPYQGAIQGPEEFVEGMALGLKAL
VGGAVGGLAGAASKITGAMAKGVAAMTMDEDYQQKRREAMNKQPAGFREGITRGGKGLVSGFVSGITGIV
TKPIKGAQKGGAAGFFKGVGKGLVGAVARPTGGIIDMASSTFQGIKRATETSEVESLRPPRFFNEDGVIR
PYRLRDGTGNQMLQVMENGRFAKYKYFTHVMINKTDMLMITRRGVLFVTKGTFGQLTCEWQYSFDEFTKE
PFIVHGRRLRIEAKERVKSVFHAREFGKIINFKTPEDARWILTKLQEAREPSPSL

The amino acid sequence of human VPS28 is shown below (UniProt Q9UK41) (SEQ ID NO: 18):

MFHGIPATPGIGAPGNKPELYEEVKLYKNAREREKYDNMAELFAVVKTMQ
ALEKAYIKDCVSPSEYTAACSRLLVQYKAAFRQVQGSEISSIDEFCRKFR
LDCPLAMERIKEDRPITIKDDKGNLNRCIADVVSLFITVMDKLRLEIRAM
DEIQPDLRELMETMHRMSHLPPDFEGRQTVSQWLQTLSGMSASDELDDSQ
VRQMLFDLESAYNAFNRFLHA

The nucleotide sequence of human VPS28 is shown below with alternate exons underlined (NCBI gene ID 51160) (SEQ ID NO: 19):

ATGTTTCATGGGATCCCAGCCACGCCGGGCATAGGAGCCCCTGGGAACAA
GCCGGAGCTGTATGAGGAAGTGAAGTTGTACAAGAACGCCCGGGAGAGGG
AGAAGTACGACAACATGGCAGAGCTGTTTGCGGTGGTGAAGACAATGCAA
GCCCTGGAGAAGGCCTACATCAAGGACTGTGTCTCCCCCAGCGAGTACAC
TGCAGCCTGCTCCCGGCTCCTGGTCCAATACAAAGCTGCCTTCAGGCAGG
TCCAGGGCTCAGAAATCAGCTCTATTGACGAATTCTGCCGCAAGTTCCGC
CTGGACTGCCCGCTGGCCATGGAGCGGATCAAGGAGGACCGGCCCATCAC
CATCAAGGACGACAAGGGCAACCTCAACCGCTGCATCGCAGACGTGGTCT
CGCTCTTCATCACGGTCATGGACAAGCTGCGCCTGGAGATCCGCGCCATG
GATGAGATCCAGCCCGACCTGCGAGAGCTGATGGAGACCATGCACCGCAT
GAGCCACCTCCCACCCGACTTTGAGGGCCGCCAGACGGTCAGCCAGTGGC
TGCAGACCCTGAGCGGCATGTCGGCGTCAGATGAGCTGGACGACTCACAG
GTGCGTCAGATGCTGTTCGACCTGGAGTCAGCCTACAACGCCTTCAACCG
CTTCCTGCATGCCTGA

The protein sequence of human VPS28 (SEQ ID NO: 18) is shown again with the amino acids encoded by alternate exons underlined. Amino acids encoded across a splice junction are in bold.

MFHGIPATPGIGAPGNKPELYEEVKLYKNAREREKYDNMAELFAVVKTMQ
ALEKAYIKDCVSPSEYTAACSRLLVQYKAAFRQVQGSEISSIDEFCRKFR
LDCPLAMERIKEDRPITIKDDKGNLNRCIADVVSLFITVMDKLRLEIRAM
DEIQPDLRELMETMHRMSHLPPDFEGRQTVSQWLQTLSGMSASDELDDSQ
VRQMLFDLESAYNAFNRFLHA

### REFERENCES

Alexandre B M, Charro N, Blonder J, Lopes C, Azevedo P, Bugalho de Almeida A, Chan K C, Prieto D A, Issaq H, Veenstra T D, Penque D. Profiling the erythrocyte membrane proteome isolated from patients diagnosed with chronic obstructive pulmonary disease. J Proteomics 2012.
An C H, Kim Y R, Kim H S, Kim S S, Yoo N J, Lee S H. Frameshift mutations of vacuolar protein sorting genes in gastric and colorectal cancers with microsatellite instability. Hum Pathol 2012; 43(1):40-47.
Foller M, Hermann A, Gu S, Alesutan I, Qadri S M, Borst O, Schmidt E M, Schiele F, vom Hagen J M, Saft C, Schols L, Lerche H, Stournaras C, Storch A, Lang F. Chorein-sensitive polymerization of cortical actin and suicidal cell death in chorea-acanthocytosis. Faseb J 2012; 26(4):1526-1534.
Farley S J. Prostate cancer: Risk stratification of PSA-based screening. Nat Revs Urol; 7: 643, 2010
Hayashi T, Kishida M, Nishizawa Y, lijima M, Koriyama C, Nakamura M, Sano A, Kishida S. Subcellular localization and putative role of VPS13A/chorein in dopaminergic neuronal cells. Biochem Biophys Res Commun 2012; 419(3):511-516.
Pineda-Molina E, Belrhali H, Piefer A J, Akula I, Bates P, Weissenhorn W. The crystal structure of the C-terminal domain of Vps28 reveals a conserved surface required for Vps20 recruitment. Traffic 2006; 7(8):1007-1016.
Rusten T E, Vaccari T, Stenmark H. Shaping development with ESCRTs. Nat Cell Biol 2012; 14(1):38-45.
Burgdorf S, Leister P, Scheidtmann K H. TSG101 interacts with apoptosis antagonizing transcription factor and enhances androgen receptor-mediated transcription by promoting its monoubiquitination. J Biol Chem 2004; 279(17):17524-17534.
Sun Z, Pan J, Hope W X, Cohen S N, Balk S P. Tumor susceptibility gene 101 protein represses androgen receptor transactivation and interacts with p300. Cancer 1999; 86(4):689-696.
Steiner T, Junker K, Burkhardt F, Braunsdorf A, Janitzky V, Schubert J. Gain in chromosome 8q correlates with early progression in hormonal treated prostate cancer. Eur Urol 2002; 41(2):167-171.
Stauch B L, Robinson M B, Forloni G, Tsai G, Coyle J T. The effects of N acetylated alpha linked acidic dipeptidase (NAALADase) inhibitors on [3H]NAAG catabolism in vivo. Neurosci Lett 1989; 100(1-3):295-300.
Liu T, Nedrow-Byers J R, Hopkins M R, Wu L Y, Lee J, Reilly P T, Berkman C E. Targeting prostate cancer cells with a multivalent PSMA inhibitor-guided streptavidin conjugate. Bioorg Med Chem Lett 2012; 22(12):3931-3934.
Osborne J R, Akhtar N H, Vallabhajosula S, Anand A, Deh K, Tagawa S T. Prostate-specific membrane antigen-based imaging. Urol Oncol 2012.
Burgner D, Davila S, Breunis W B, Ng S B, Li Y, Bonnard C, Ling L, Wright V J, Thalamuthu A, Odam M, Shimizu C, Burns J C, Levin M, Kuijpers T W, Hibberd M L. A genome-wide association study identifies novel and functionally related susceptibility Loci for Kawasaki disease. PLoS Genet 2009; 5(1):e1000319.
Tonkin E T, Smith M, Eichhorn P, Jones S, Imamwerdi B, Lindsay S, Jackson M, Wang T J, Ireland M, Burn J, Krantz I D, Carr P, Strachan T. A giant novel gene undergoing extensive alternative splicing is severed by a Cornelia de Lange-associated translocation breakpoint at 3q26.3. Hum Genet 2004; 115(2):139-148.
Whitaker H C, Kote-Jarai Z, Ross-Adams H, Warren A Y, Burge J, George A, Bancroft E, Jhavar S, Leongamornlert D, Tymrakiewicz M, Saunders E, Page E, Mitra A, Mitchell C, Lindeman G J, Evans D G, Blanco I, Mercer C, Rubinstein W S, Clowes V, Douglas F, Hodgson S, Walker L,
Donaldson A, Izatt L, Dorkins H, Male A, Tucker K, Stapleton A, Lam J, Kirk J, Lilja H, Easton D, Cooper C, Eeles R, Neal D E. The rs10993994 risk allele for prostate cancer results in clinically relevant changes in microseminoprotein-beta expression in tissue and urine. PLoS One 2010; 5(10):e13363.
Cuzick J, Fisher G, Kattan M W, Berney D, Oliver T, Foster C S, Moller H, Reuter V, Fearn P, Eastham J, Scardino P. Long-term outcome among men with conservatively treated localised prostate cancer. Br J Cancer 2006; 95(9):1186-1194.
Thirkettle H J, Mills I G, Whitaker H C, Neal D E. Nuclear LYRIC/AEG-1 interacts with PLZF and relieves PLZF-mediated repression. Oncogene 2009; 28(41):3663-3670.
Whitaker H C, Stanbury D P, Brinham C, Girling J, Hanrahan S, Totty N, Neal D E. Labeling and identification of LNCaP cell surface proteins: a pilot study. Prostate 2007; 67(9):943-954.
Ayala A G, Ro J Y. Prostatic intraepithelial neoplasia: recent advances. Arch Pathol Lab Med 2007; 131: 1257-1256.
Montironi R, Mazzucchelli R, Lopez-Beltran A, Scarpelli M, Cheng L. BJU Int. 2011; 108(9):1394-1401.
Bostwick D G, Meiers I. Atypical small acinar proliferation in the prostate: clinical significance in 2006. Arch Pathol Lab Med. July 2006; 130(7): 952-957.
Montironi R, Scattoni V, Mazzucchelli R, Lopez-Beltran A, Bostwick D G, Montorsi F. Atypical foci suspicious but not diagnostic of malignancy in prostate needle biopsies (also referred to as “atypical small acinar proliferation suspicious for but not diagnostic of malignancy”). Eur Urol. October 2006; 50(4):666-674.
Schlesinger C, Bostwick D G, Iczkowski K A. High-grade prostatic intraepithelial neoplasia and atypical small acinar proliferation: predictive value for cancer in current practice. Am J Surg Pathol. 2005; 29(9):1201-7
Wolf, A. M., Wender, R. C., Etzioni, R. B., Thompson, I. M., D'Amico, A. V., Volk, R. J., Brooks, D. D., Dash, C., Guessous, I., Andrews, K., DeSantis, C., and Smith, R. A. (2010) CA Cancer J Clin 60, 70-98
Salagierski, M., and Schalken, J. A. (2012) J Urol 187, 795-801
Whitaker, H. C., Kote-Jarai, Z., Ross Adams, H., Warren, A. Y., Burge, J., George, A., Bancroft, E., Jhavar, S., Leongamornlert, D., Tymrakiewicz, M., Saunders, E., Page, E., Mitra, A., Mitchell, G., Lindeman, G. J., Evans, D. G., Blanco, I., Mercer, C., Rubinstein, W. S., Clowes, V., Douglas, F., Hodgson, S., Walker, L., Donaldson, A., Izatt, L., Dorkins, H., Male, A., Tucker, K., Stapleton, A., Lam, J., Kirk, J., Lilja, H., Easton, D., Cooper, C., Eeles, R., and Neal, D. E. (2010) PLoS One 5, e13363
Schwarzenbach H, Hoon D S, Pantel K (2011). Cell free nucleic acids as biomarkers in cancer patients. Nat Rev Cancer 11: 426-437.
Leon S A, Shapiro B, Sklaroff D M, Yaros M J (1977). Free DNA in the serum of cancer patients and the effect of therapy. Cancer Res 37: 646-650.
Papadopoulou E, Davilas E, Sotiriou V, Georgakopoulos E, Georgakopoulou S, Koliopanos A et al (2006). Cell-free DNA and RNA in plasma as a new molecular marker for prostate and breast cancer. Ann N Y Acad Sci 1075: 235-243.
Rainen L, Oelmueller U, Jurgensen S, Wyrich R, Ballas C, Schram J et al (2002). Stabilization of mRNA expression in whole blood samples. Clin Chem 48: 1883-1890.
Batliwalla F M, Li W, Ritchlin C T, Xiao X, Brenner M, Laragione T et al (2005). Microarray analyses of peripheral blood cells identifies unique gene expression signature in psoriatic arthritis. Mol Med 11: 21-29.
Lewis D A, Stashenko G J, Akay O M, Price L I, Owzar K, Ginsburg G S et al (2011). Whole blood gene expression analyses in patients with single versus recurrent venous thromboembolism. Thromb Res 128: 536-540
Li D, Butt A, Clarke S, Swaminathana R (2004). Real-time quantitative PCR measurement of thyroglobulin mRNA in peripheral blood of thyroid cancer patients and healthy subjects. Ann N Y Acad Sci 1022: 147-151.
Yang B, Xu Q, Wu F, Liu F, Ye X, Liu G C et al (2011). Using peripheral blood mRNA signature to distinguish between breast cancer and benign breast disease in non-conclusive mammography patients. Cancer Biol Ther 10: 1235-1239.
Epstein et al. (2005) The 2005 International Society of Urological Pathology (ISUP) Consensus Conference on Gleason Grading of Prostatic Carcinoma. Am J Surg Pathol 2005 September; 29:1228-42

1. A method for predicting the prognosis of a subject with prostate cancer, said method comprising detecting whether a test sample obtained from the subject expresses:
(i) a gene encoding a VPS13A protein, a gene encoding an NAALADL2 protein and/or a gene encoding a VPS28 protein; or
(ii) a VPS13A protein, an NAALADL2 protein and/or a VPS28 protein;
at a level higher than the expression of the respective gene(s) or protein(s) in a normal reference sample, wherein a higher level of expression and/or activity of the respective gene(s) or protein in a normal reference sample compared to in the test sample is indicative of a poor prognosis.

2. A method according to claim 1, wherein the method comprises detecting whether the test sample obtained from the subject expresses:
(i) a gene encoding a VPS13A protein and a gene encoding an NAALADL2 protein; or
(ii) a VPS13A protein and an NAALADL2 protein;
at a level higher than the expression of the respective genes or proteins in a normal reference sample, wherein a higher level of expression and/or activity of the respective genes or proteins in a normal reference sample compared to in the test sample is indicative of a poor prognosis.

3. A method according to claim 1, wherein the method comprises detecting whether the test sample obtained from the subject expresses:
(i) a gene encoding a VPS13A protein, a gene encoding an NAALADL2 protein, and a gene encoding a VPS28 protein;
i) a VPS13A protein, an NAALADL2 protein and a VPS28 protein;
at a level higher than the expression of the respective genes or proteins in a normal reference sample, wherein a higher level of expression and/or activity of the respective genes or proteins in a normal reference sample compared to in the test sample is indicative of a poor prognosis.

4. A method according to claim 1, wherein increased expression and/or activity of one or more of the respective genes or proteins is predictive of a decreased progression free survival time.

5-23. (canceled)

24. A method according to claim 1, wherein increased expression and/or activity of one or more of the respective genes or proteins is indicative of an increased likelihood of clinical or biochemical relapse following radical prostatectomy.

25. A method according to claim 1, wherein the level of expression and/or activity of the respective gene(s) or protein(s) in the test sample compared to the normal reference sample is indicative of the grade of prostate cancer in the subject, such that higher expression and/or activity of the respective gene(s) or protein(s) is indicative of a higher grade of prostate cancer, and optionally indicates that the subject is likely to have prostate cancer with a Gleason grade of at least 3+3, 3+4 or 4+3.

26. A method according to claim 1, wherein a higher level of expression or activity of the respective genes or proteins in the test sample compared to the normal reference sample is indicative of the pathological stage of prostate cancer in the subject, such that higher expression and/or activity of the respective gene(s) or protein(s) is indicative of a higher pathological stage of prostate cancer, and optionally indicates that the subject is likely to have prostate cancer with a pathological stage of pT2 or pT3.

27. A method for monitoring progression of prostate cancer in a subject, said method comprising determining whether a test sample obtained from the subject expresses:
(i) a gene encoding a VPS13A protein, a gene encoding an NAALADL2 protein and/or a gene encoding a VPS28 protein; or
(ii) a VPS13A protein, an NAALADL2 protein and/or a VPS28 protein;
at a level higher than the expression of the respective gene(s) or protein(s) in a previous sample obtained from said subject, wherein a higher level of expression and/or activity of the respective gene(s) or protein(s) in the test sample compared to the previous sample is indicative of progression of prostate cancer to a more aggressive form, and is optionally indicative of progression of prostate cancer to a Gleason grade of at least 3+3, 3+4 or 4+3.

28. A method according to claim 27, wherein the method comprises determining whether the test sample obtained from the subject expresses:
(i) a gene encoding a VPS13A protein and a gene encoding an NAALADL2 protein; or
(ii) a VPS13A protein and an NAALADL2 protein;
at a level higher than the expression of the respective genes or proteins in a previous sample obtained from said subject, wherein a higher level of expression and/or activity of the respective genes or proteins in the test sample compared to in the previous sample is indicative of progression of prostate cancer to a more aggressive form.

29. A method according to claim 27, wherein the method comprises determining whether the test sample obtained from the subject expresses:
(i) a gene encoding a VPS13A protein, a gene encoding an NAALADL2 protein and a gene encoding a VPS28 protein; or
(ii) a VPS13A protein, an NAALADL2 protein and a VPS28 protein;
at a level higher than the expression of the respective genes or proteins in a previous sample obtained from said subject, wherein a higher level of expression and/or activity of the respective genes or proteins in the test sample compared to in the previous sample is indicative of progression of prostate cancer to a more aggressive form.

30. A method according to claim 1, wherein the test sample is whole blood, plasma, serum, urine, ejaculate, stool, tissue or cells from a pancreatic biopsy, a prostate biopsy, tissue or cells from a radical prostatectomy or a biliary pancreatic sponge.

31. A method according to claim 27, wherein the test sample is whole blood, plasma, serum, urine, ejaculate, stool, tissue or cells from a pancreatic biopsy, a prostate biopsy, tissue or cells from a radical prostatectomy or a biliary pancreatic sponge.

32. A method according to claim 1, wherein if the test sample has a higher level of expression and/or activity of one or more of the respective genes or proteins, the subject is selected for surgery, chemotherapy and/or radiation.

33. A method according to claim 27, wherein if the test sample has a higher level of expression and/or activity of one or more of the respective genes or proteins, the subject is selected for surgery, chemotherapy and/or radiation.


SEQUENCE LISTING (19 sequences)

SEQ ID NO: 1 - DNA, 24 bp, Artificial sequence
  Synthetic sequence Forward primer
acgcaccctgtctgactacaacat

SEQ ID NO: 2 - DNA, 24 bp, Artificial sequence
  Synthetic sequence Reverse primer
agggatgccttccttgtcttggat

SEQ ID NO: 3 - DNA, 24 bp, Artificial sequence
  Synthetic sequence Forward primer
aagaagatcttggacagcgtgggt

SEQ ID NO: 4 - DNA, 24 bp, Artificial sequence
  Synthetic sequence Reverse primer
taccctgggcaatgacgtcttcaa

SEQ ID NO: 5 - DNA, 19 bp, Artificial sequence
  Synthetic sequence Forward primer
gaaggtgaaggtcggagtc

SEQ ID NO: 6 - DNA, 19 bp, Artificial sequence
  Synthetic sequence Reverse primer
aagatggtgatgggatttc

SEQ ID NO: 7 - DNA, 24 bp, Artificial sequence
  Synthetic sequence Forward primer
tagttggtggagctgttggtggat

SEQ ID NO: 8 - DNA, 24 bp, Artificial sequence
  Synthetic sequence Reverse primer
tttcctccacgagtgatgccttct

SEQ ID NO: 9 - DNA, 21 bp, Artificial sequence
  Synthetic sequence Forward primer
gcctagaggatgtttcatggg

SEQ ID NO: 10 - DNA, 20 bp, Artificial sequence
  Synthetic sequence Reverse primer
tgtcgtacttctccctctcc

SEQ ID NO: 11 - DNA, 24 bp, Artificial sequence
  Synthetic sequence Forward primer
tgactctgagcagcagtggtcaat

SEQ ID NO: 12 - DNA, 24 bp, Artificial sequence
  Synthetic sequence Reverse primer
agccttgagagttcctttggcaga

SEQ ID NO: 13 - protein, 795 aa, Homo sapiens
MGENEASLPNTSLQGKKMAYQKVHADQRAPGHSQYLDNDDLQATALDLEWDMEKELEESGFDQFQLDGAENQNLGHSETIDLNLDSIQPATSPKGRFQRLQEESDYITHYTRSAPKSNRCNFCHVLKILCTATILFIFGILIGYYVHTNCPSDAPSSGTVDPQLYQEILKTIQAEDIKKSFRNLVQLYKNEDDMEISKKIKTQWTSLGLEDVQFVNYSVLLDLPGPSPSTVTLSSSGQCFHPNGQPCSEEARKDSSQDLLYSYAAYSAKGTLKAEVIDVSYGMADDLKRIRKIKNVTNQIALLKLGKLPLLYKLSSLEKAGFGGVLLYIDPCDLPKTVNPSHDTFMVSLNPGGDPSTPGYPSVDESFRQSRSNLTSLLVQPISAPLVAKLISSPKARTKNEACSSLELPNNEIRVVSMQVQTVTKLKTVTNVVGFVMGLTSPDRYIIVGSHHHTAHSYNGQEWASSTAIITAFIRALMSKVKRGWRPDRTIVFCSWGGTAFGNIGSYEWGEDFKKVLQKNVVAYISLHSPIRGNSSLYPVASPSLQQLVVEKNNFNCTRRAQCPETNISSIQIQGDADYFINHLGVPIVQFAYEDIKTLEGPSFLSEARFSTRATKIEEMDPSFNLHETITKLSGEVILQIANEPVLPFNALDIALEVQNNLKGDQPNTHQLLAMALRLRESAELFQSDEMRPANDPKERAPIRIRMLNDILQDMEKSFLVKQAPPGFYRNILYHLDEKTSRFSILIEAWEHCKPLASNETLQEALSEVLNSINSAQVYFKAGLDVFKSVLDGKN

SEQ ID NO: 14 - DNA, 1715 bp, Homo sapiens
gggtcagtagaaagtcagaaggtcacaaagcttgcagggtaagtgacacaacttgaaactgcttggccctctttaaaaagaaataataaaatgggagagaatgaagcaagtttacctaacacgtctttgcaaggtaaaaagatggcctatcagaaggtccatgcagatcaaagagctccaggacactcacagtacttagacaatgatgaccttcaagccactgcccttgacttagagtgggacatggagaaggaactagaggagtctggttttgaccaattccagctagacagtgctgagaatcagaacctagggcattcagagactatagacctcaatcttgattccattcaaccagcaacttcacccaaaggaaggttccagagacttcaagaagaatctgactacattacccattatacacgatctgcaccaaagagcaatcgctgcaacttttgccacgtcttaaaaatgctttgcacagccaccattttatttatttttgggattttgataggttattatgtacatacaaattgcccttcagatgctccatcttcaggaacagttgatcctcagttatatcaagagattctcaagacaatccaggcagaagatattaagaagtctttcagaaatttggtacaactatataaaaatgaagatgacatggaaatttcaaagaagattaagactcagtggacctctttgggcctagaagatgtacagtttgtaaattactctgtgctgcttgatctgccaggcccttctcccagcactgtgactctgagcagcagtggtcaatgctttcatcctaatggccagccttgcagtgaagaagccagaaaagatagcagccaagacctgctctattcatatgcagcctattctgccaaaggaactctcaaggctgaagtcatcgatgtgagttatggaatggcagatgatttaaaaaggattaggaaaataaaaaacgtaacaaatcagatcgcactcctgaaattaggaaaattgccactgctttataaggttggtccagtgaatgttattcagtggtttggtcaatattttgccttgttttgttggaattatatgcttttgtgagtgtggagtgtgtgtgtgcatataggtgtgtgagagagagaaggggagaggaagaaagagaggcagagagtgtcacagaaagatggcttttccacattagaacattttaatttaagatatttaagaacaatatatttatgcccttatttctttagagagaaaataccttaagtcaggtaacactgagtttgtgggaccttaataaaattggcatactcttcataatggtacctatctggaatagtaaaaatagagaaccaccctgtgttcatcttatgacatatgtgaaacttctaatctattatcaaatggactaattatcatgttctctatgttagacaagtatctagatgatttacaccctttagtgattattttgtcaactatacaactacagttactaactgtgatcaggattttaattaaaatataattgctaagagtagcagaattttgatttattttatttgaatggaagtttattaacactcatccacagatacacttatgtaattaagtttctgatgatgaaccagcacaatagacagccactactgctcattctcgcttcatttcctttttctatttaaaaaaaaaaaaaaaaaaaaaaa

SEQ ID NO: 15 - protein, 3174 aa, Homo sapiens
MVFESVVVDVLNRFLGDYVVDLDTSQLSLGIWKGAVALKNLQIKENALSQLDVPFKVKVGHIGNLKLIIPWKNLYTQPVEAVLEEIYLLIVPSSRIKYDPLKEEKQLMEAKQQELKRIEEAKQKVVDQEQHLPEKQDTFAEKLVTQIIKNLQVKISSIHIRYEDDITNRDKPLSFGISLQNLSMQTTDQYWVPCLHDETEKLVRKLIRLDNLFAYWNVKSQMFYLSDYDNSLDDLKNGIVNENIVPEGYDFVFRPISANAKLVMNRRSDFDFSAPKINLEIELHNIAIEFNKPQYFSIMELLESVDMMAQNLPYRKFKPDVPLHHHAREWWAYAIHGVLEVNVCPRLWMWSWKHIRKHRQKVKQYKELYKKKLTSKKPPGELLVSLEELEKTLDVFNITIARQTAEVEVKKAGYKIYKEGVKDPEDNKGWFSWLWSWSEQNTNEQQPDVQPETLEEMLTPEEKALLYEAIGYSETAVDPTLLKTFEALKFFVHLKSMSIVLRENHQKPELVDIVIEEFSTLIVQRPGAQAIKFETKIDSFHITGLPDNSEKPRLLSSLDDAMSLFQITFEINPLDETVSQRCIIEAEPLEIIYDARTVNSIVEFFRPPKEVHLAQLTAATLTKLEEFRSKTATGLLYIIETQKVLDLKINLKASYIIVPQDGIFSPTSNLLLLDLGHLKVTSKSRSELPDVKQGEANLKEIMDRAYDSFDIQLTSVQLLYSRVGDNWREARKLSVSTQHILVPMHFNLELSKAMVFMDVRMPKFKIYGKLPLISLRISDKKLQGIMELIESIPKPEPVTEVSAPVKSFQIQTSTSLGTSQISQKIIPLLELPSVSEDDSEEEFFDAPCSPLEEPLQFPTGVKSIRTRKLQKQDCSVNMTTFKIRFEVPKVLIEFYHLVGDCELSVVEILVLGLGAEIEIRTYDLKANAFLKEFCLKCPEYLDENKKPVYLVTTLDNTMEDLLTLEYVKAEKNVPDLKSTYNNVLQLIKVNFSSLDIHLHTEALLNTINYLHNILPQSEEKSAPVSTTETEDKGDVIKKLALKLSTNEDIITLQILAELSCLQIFIQDQKCNISEIKIEGLDSEMIMRPSETEINAKLRNIIVLDSDITAIYKKAVYITGKEVFSFKMVSYMDATAGSAYTDMNVVDIQVNLIVGCIEVVFVTKFLYSILAFIDNFQAAKQALAEATVQAAGMAATGVKELAQRSSRMALDINIKAPVVVIPQSPVSENVFVADFGLITMTNTFHMITESQSSPPPVIDLITIKLSEMRLYRSRFINDAYQEVLDLLLPLNLEVVVERNLCWEWYQEVPCFNVNAQLKPMEFILSQEDITTIFKTLHGNIWYEKDGSASPAVTKDQYSATSGVTTNASHHSGGATVVTAAVVEVHSRALLVKTTLNISFKTDDLTMVLYSPGPKQASFTDVRDPSLKLAEFKLENIISTLKMYTDGSTFSSFSLKNCILDDKRPHVKKATPRMIGLTVGFDKKDMMDIKYRKVRDGCVTDAVFQEMYICASVEFLQTVANVFLEAYTTGTAVETSVQTWTAKEEVPTQESVKWEINVIIKNPEIVFVADMTKNDAPALVITTQCEICYKGNLENSTMTAAIKDLQVRACPFLPVKRKGKITTVLQPCDLFYQTTQKGTDPQVIDMSVKSLTLKVSPVIINTMITITSALYTTKETIPEETASSTAHLWEKKDTKTLKMWFLEESNETEKIAPTTELVPKGEMIKMNIDSIFIVLEAGIGHRTVPMLLAKSRFSGEGKNWSSLINLHCQLELEVHYYNEMFGVWEPLLEPLEIDQTEDFRPWNLGIKMKKKAKMAIVESDPEEENYKVPEYKTVISFHSKDQLNITLSKCGLVMLNNLVKAFTEAATGSSADFVKDLAPFMILNSLGLTISVSPSDSFSVLNIPMAKSYVLKNGESLSMDYIRTKDNDHFNAMTSLSSKLFFILLTPVNHSTADKIPLTKVGRRLYTVRHRESGVERSIVCQIDTVEGSKKVTIRSPVQIRNHFSVPLSVYEGDTLLGTASPENEFNIPLGSYRSFIFLKPEDENYQMCEGIDFEEIIKNDGALLKKKCRSKNPSKESFLINIVPEKDNLTSLSVYSEDGWDLPYIMHLWPPILLRNLLPYKIAYYIEGIENSVFTLSEGHSAQICTAQLGKARLHLKLLDYLNHDWKSEYHIKPNQQDISFVSFTCVTEMEKTDLDIAVHMTYNTGQTVVAFHSPYWMVNKTGRMLQYKADGIHRKHPPNYKKPVLFSFQPNHFFNNNKVQLMVTDSELSNQFSIDTVGSHGAVKCKGLKMDYQVGVTIDLSSFNITRIVTFTPFYMIKNKSKYHISVAEEGNDKWLSLDLEQCIPFWPEYASSKLLIQVERSEDPPKRIYFNKQENCILLRLDNELGGIIAEVNLAEHSTVITFLDYHDGAATFLLINHTKNELVQYNQSSLSEIEDSLPPGKAVFYTWADPVGSRRLKWRCRKSHGEVTQKDDMMMPIDLGEKTIYLVSFFEGLQRIILFTEDPRVFKVTYESEKAELAEQEIAVALQDVGISLVNNYTKQEVAYIGITSSDVVWETKPKKKARWKPMSVKHTEKLEREFKEYTESSPSEDKVIQLDTNVPVRLTPTGHNMKILQPHVIALRRNYLPALKVEYNTSAHQSSFRIQIYRIQIQNQIHGAVFPFVFYPVKPPKSVTMDSAPKPFTDVSIVMRSAGHSQISRIKYFKVLIQEMDLRLDLGFIYALTDLMTEAEVTENTEVELFHKDIEAFKEEYKTASLVDQSQVSLYEYFHISPIKLHLSVSLSSGREEAKDSKQNGGLIPVHSLNLLLKSIGATLTDVQDVVFKLAFFELNYQFHTTSDLQSEVIRHYSKQAIKQMYVLILGLDVLGNPFGLIREFSEGVEAFFYEPYQGAIQGPEEFVEGMALGLKALVGGAVGGLAGAASKITGAMAKGVAAMTMDEDYQQKRREAMNKQPAGFREGITRGGKGLVSGFVSGITGIVTKPIKGAQKGGAAGFFKGVGKGLVGAVARPTGGIIDMASSTFQGIKRATETSEVESLRPPRFFNEDGVIRPYRLRDGTGNQMLQVMENGRFAKYKYFTHVMINKTDMLMITRRGVLFVTKGTFGQLTCEWQYSFDEFTKEPFIVHGRRLRIEAKERVKSVFHAREFGKIINFKTPEDARWILTKLQEAREPSPSL

SEQ ID NO: 16 - DNA, 9408 bp, Homo sapiens
atggttttcgagtcggtggtcgtggacgtgttgaaccggttcttgggggactatgtggtggacttggacacgtcccagctctctctgggcatctggaaaggagctgtggccctcaagaatcttcaaattaaagaaaatgccctgagtcaactggatgtaccatttaaagttaaagttggtcacataggtaatcttaaacttataattccatggaaaaacctttatactcaacctgttgaagccgtattggaagaaatttatttacttatagtgccttcttctagaataaaatatgatcctttaaaagaagagaaacaactcatggaagcaaagcaacaggaactgaaaagaatagaagaagcaaaacaaaaagtagttgatcaagaacaacatctgccggaaaaacaggacacttttgcagaaaaattagttacacagatcataaaaaatcttcaggtgaaaatttccagtatccatattcgttatgaagatgatatcacaaatcgggacaaaccgctgtcatttggtatttcccttcaaaatctgagcatgcagacaactgatcaatactgggttccatgtttacatgatgaaactgagaaactggttcgtaagttaatccgattggataacctgtttgcctattggaatgtgaagtctcagatgttttatcttagtgattatgataactccttggacgacttgaagaatggcattgtcaatgaaaatattgttccagaaggttatgattttgtatttcgtcccatatctgctaatgccaaacttgtgatgaatcgccgatctgattttgacttttctgcccccaaaataaacttggaaattgagttacataacatagcaattgaatttaataaaccacagtatttcagtattatggagcttcttgaatcagttgatatgatggcacaaaatctgccatataggaagttcaaacctgatgtgcctcttcaccaccatgccagagaatggtgggcttatgctatacatggcgttcttgaagtaaatgtttgccccaggttatggatgtggtcatggaagcatattagaaaacataggcaaaaagtgaagcaatataaagaactgtataaaaaaaagttaacaagtaagaagccacctggtgaacttctcgtgtctttggaggagttggaaaaaaccttggatgtctttaatataactatagctagacagacggcagaagttgaggtaaagaaagctggatacaaaatttacaaagaaggagtaaaagatccagaggataataaagggtggtttagctggctatggtcttggtcagaacaaaatactaatgaacagcaaccagatgttcaacctgaaactcttgaagaaatgttgacacctgaagaaaaagctttactctatgaagcaattggctatagtgaaacagcagttgatccaactttactaaaaacatttgaagccttgaagttttttgtccacttgaaaagtatgtctattgttctaagagaaaatcatcaaaaacctgagctggtagatattgtaatagaagaatttagcaccttaattgtgcaaagaccaggagcacaagcaataaaatttgaaactaaaatagattcatttcatattactggcttaccagataattcagaaaaaccccgcctcctgtcttcattggatgatgcaatgtcacttttccaaattacatttgagataaatccattagatgaaactgtttctcagaggtgtatcatagaagctgaacctttagaaatcatatatgatgcaaggacagtgaatagtatagtggaattcttcagacctccaaaagaggtacatctagcacagctcactgcagcaactttgacaaaactggaagaatttcgcagtaagacagcaacaggtctactgtatattattgaaacacagaaagttcttgatctcaaaattaatttgaaggcttcatatattattgtcccacaagatggaatttttagtcctacatcaaatctgcttcttttggaccttggtcatctaaaggtgacgagtaaaagtcgttctgaattaccagatgtgaaacaaggtgaggccaatcttaaagagataatggatagagcttatgattcatttgatattcaacttacaagtgtacagctgctttacagtagagttggtgataattggagagaagcacgaaaactcagtgtatctacccagcatattttggtacccatgcacttcaatttggaactgtctaaggccatggttttcatggatgtaaggatgcccaaattcaagatttatggaaagttacctcttatttctttacgaatctcagataaaaaactacaagggattatggaattgattgaaagcattccaaaacctgaaccagtaactgaagtatctgcccctgtcaaatcattccagattcaaacatctacttctttgggaacatcacagatttcacagaaaataattcctctcttggaacttccatctgtttctgaagatgattcagaggaggaattttttgatgcaccatgtagtcccttggaagaacctcttcagtttccaactggagttaaaagtattcgaaccagaaagttacaaaagcaggattgttcagtaaatatgactacatttaaaataagatttgaagtaccaaaggttttgatcgagttttatcaccttgttggagattgtgaactatctgtggtagaaattcttgttttaggattgggtgcagaaattgagattagaacatacgatttgaaagcaaatgcctttttgaaagagttctgcttaaaatgcccagaatacttggatgaaaacaagaaaccagtttatttggttacaaccctggataacacaatggaagacctgttaacgctggaatatgtaaaggctgaaaagaatgtacccgacttgaaaagtacctataacaatgttttacaattgattaaggtaaatttttcctctttggatattcatttacacactgaagcacttctgaatacaataaattatcttcataatatccttccgcaatcagaggaaaaatcagccccagtgtccactacagagactgaagacaaaggagatgtcattaaaaaattagggcttgattctgagatgattatgaggccttcagaaactgaaataaacgcaaagctaaggaatataattgttttagattctgatataacagctatatacaaaaaggctgtttatatcactggaaaagaagttttcagcttcaaaatggtttcttacatggatgcaactgctggttctgcatacacagatatgaatgtggttgacattcaggttaatttaatagttggttgcattgaagtagtttttgtcacgaaatttctatattctatattggcttttatagataattttcaggcagctaaacaagccttggctgaggcaactgttcaggcagctggaatggctgctactggtgtaaaagaactcgcacaaaggagttccagaatggcactggatattaacatcaaagccccagttgtggtcatcccgcagtctccagtttctgaaaatgtttttgttgctgattttggactaattacaatgacaaatacctttcatatgataacagagagccagagctctcccccacctgttattgatttgataacaataaagctgagtgaaatgcgactatacagatctcgatttattaatgatgcataccaggaagtactggatctactcctgccattaaatcttgaggttgtggttgaacgaaatttatgctgggagtggtaccaggaagttccttgttttaatgtaaatgctcagctgaaaccaatggagttcattcttagtcaagaagatataacaactatttttaaaacattgcatggcaatatatggtatgaaaaagatggtagtgcctcacctgctgtaacaaaagaccaatacagtgccactagtggagttactactaatgcttcacaccattcaggaggagcaactgtggtgacagctgctgtggtagaagtacattcacgtgccttactagttaagacaacactaaacataagcttcaaaactgatgatctcaccatggtgctgtatagtccaggtcctaaacaggcttcctttacagatgttcgtgatccttctctgaaacttgctgaatttaaattggagaatattataagtactttaaaaatgtatacagatggctcaacattttcttccttctcattaaaaaactgtattttagatgataaaagacctcatgtcaagaaagcaactcctcgaatgataggactgacagttggttttgacaaaaaagacatgatggatataaagtacaggaaagtcagagatggttgtgtgactgatgcggtctttcaagaaatgtatatttgtgcaagcgtagaatttctgcagactgttgcaaatgtctttcttgaggcctacaccacaggcactgctgtagaaaccagtgtgcaaacatggactgctaaggaagaagtacctacacaggaatcagtgaagtgggaaattaatgttattattaaaaatcctgaaattgtgtttgtagctgacatgacaaaaaatgatgctcctgctttagtcattacaacacaatgtgaaatttgctataaaggtaaccttgaaaatagtacaatgactgctgccattaaagatctccaagtgagagcctgcccgtttcttccagtcaagagaaaaggcaaaatcactactgttttgcagccctgtgacttgttttatcaaactactcagaaaggtacagatccacaagtgatcgatatgtcagtaaaatccctgacactaaaggtttcaccagttattataaatactatgattaccataacttcagcactgtatacaactaaggaaaccatcccagaagaaacggcttcttctactgcacatttatgggaaaagaaggatacaaagactttaaaaatgtggtttcttgaagaatcaaatgaaactgaaaaaatagctcccacaactgaattggtacccaaaggcgagatgataaaaatgaacattgattctatttttatagttcttgaggctggaattggtcatagaacagtacctatgcttctggcaaagtcacgtttttcaggggaaggcaaaaactggagttccctaataaatctgcactgtcagcttgagctagaagtgcattattataatgaaatgtttggtgtatgggagcctttgcttgaacccttagaaattgatcagactgaggattttagaccatggaatcttggtatcaagatgaaaaagaaagcaaaaatggccattgttgagtcagatcctgaagaagaaaactacaaagtgccagaatataaaactgtcatcagtttccattcaaaagaccaattaaacattacattatccaaatgtggtcttgtaatgttaaacaatttagtcaaggcatttacagaagctgccactggatcttcagctgacttcgtaaaggatctagcaccatttatgattttaaattcccttggacttactatttctgtttcgccaagtgattcttttagtgtactcaacattcctatggcaaaatcatatgtattgaaaaatggagaaagtttaagtatggattatatccgaaccaaggacaatgatcatttcaatgcaatgaccagcctaagcagcaaactcttcttcattcttcttacacctgttaaccattctactgctgataagattcctttaacaaaagtgggacgacgtctgtacactgtaagacacagagagtctggcgttgaaagatctattgtttgtcaaattgatacagtagaaggaagtaagaaggtcacaattcgctccccagtgcagataagaaatcatttttcagtcccactgtctgtttacgaaggggataccttattgggaactgcctcacctgaaaatgaattcaacataccattaggatcttaccgatcattcatttttctgaagccagaagatgagaactatcaaatgtgtgaaggaattgactttgaagagattataaaaaatgatggtgctcttctaaagaagaaatgtagatctaaaaacccttctaaggaatcatttctcattaatattgttccagaaaaagataatttaacatctctatcagtgtattcagaagatggttgggatttaccatacataatgcatttgtggccacctatcctgctccgaaatcttcttccttacaaaattgcttattatatagagggaattgaaaattcggtttttactctaagtgaaggacattcagcccagatttgtactgcacagttgggtaaagccaggctacatttaaaattacttgactatctcaatcacgattggaaaagtgaatatcacataaagcctaatcagcaagacattagttttgtcagttttacttgtgttacagaaatggaaaagactgatttagatattgctgtccatatgacttacaatactggtcagacagttgtggcatttcatagtccttattggatggtcaataaaactggccgcatgttacagtacaaagcagacggaattcatcgaaagcatccacctaattataaaaagccagttctcttttcttttcagccaaatcacttttttaataacaataaggttcaacttatggtaactgatagtgagttgtccaatcagttttcaattgatactgttggtagtcatggagctgttaaatgtaaaggcctgaaaatggactatcaagttggtgtcactatagacctgagcagttttaacattactagaattgtgacatttacccctttttatatgattaaaaacaaaagcaaataccatatatcagtggctgaagaaggaaatgataaatggctctctcttgatttggagcagtgtatccccttttggcctgagtatgcttctagtaaacttcttattcaagtcgaaaggagtgaagatcctcccaaaaggatatattttaacaagcaggaaaattgtattctattgcgtctagataacgagcttggaggtattatagcagaagtgaatttggccgagcattctacagttattacatttttagattatcatgatggagcagctacattcctcttaataaatcacacaaagaatgaacttgttcaatacaatcaaagttctctcagtgaaatagaagattccctccctcctggtaaagccgtgttttatacatgggctgatccggtgggctctagaaggctgaagtggagatgtagaaaaagccatggtgaagtaacacagaaggatgatatgatgatgcctatagatttgggggaaaagacaatatatttagtttcattctttgaaggtttacaacgcattattttattcactgaagatccaagggtatttaaagtaacatatgaaagtgagaaagcagagttagcagagcaagaaattgcagtggcattacaagatgttggaatttctcttgtcaacaattacacgaagcaagaagtagcctatataggcattacaagttctgatgtggtttgggaaacaaagcccaagaagaaggcaagatggaagccaatgagtgtaaagcacactgagaagttagagagagaatttaaggaatatactgaatcttctccttcagaagataaggttattcagttggacactaatgttccggttcgcctaacccctactggtcataacatgaaaattctgcagccgcatgtaatagctctacgaagaaattatcttccagcattaaaagtggaatataacacatctgcacatcaatcatcatttagaattcagatttacagaatacagatccaaaatcagatacatggtgctgtatttccctttgtgttttatcctgttaaacctccaaagtcggtcaccatggattcagcaccaaagccctttacagatgtcagtattgtcatgagatctgcaggacattcccagatatcacgtattaagtatttcaaagtattgattcaagaaatggatctcaggttagatcttgggtttatctatgctttaacagaccttatgacagaagctgaggtgactgaaaatacagaggttgagctttttcataaagatatagaagctttcaaagaagaatataaaacagcctcattagtagatcaatcacaagtcagcctctatgaatattttcatatatctcctatcaagttacatttaagtgtttcactgagttccggcagagaagaagctaaagattcaaaacaaaatggaggactgattccagttcattctttaaatcttttgctgaagagtattggtgccacactgacagatgtacaagatgtagtttttaagcttgcattttttgaactcaactatcagttccatacaacatccgatctacagtctgaagtcataagacactattcaaaacaggccattaagcagatgtatgtactcattcttggacttgatgttttgggaaatccatttggcttaattagagaattttctgaaggtgtagaagcatttttttatgaaccttaccagggagccatccagggtcctgaagagtttgtggaaggaatggcactaggacttaaggcactagttggtggagctgttggtggattggctggtgctgcctccaaaatcaccggtgctatggctaagggggtagcagctatgaccatggatgaagactaccaacagaagagaagagaagccatgaataagcaaccagctggttttagagaaggcatcactcgtggaggaaaaggcttagtttctggatttgttagtggcataacaggaattgttacaaaaccaatcaaaggagctcaaaaaggaggagcagctggtttctttaaaggtgttgggaaaggtttagtaggagcggtagcaaggccaactggaggcatcatagacatggctagcagtacatttcagggaataaaaagagctacagagacttctgaagtggagagtctgcgacctcctcggttcttcaatgaagatggagttatcagaccgtacaggttgagggatgggactggaaatcaaatgttacaggtcatggaaaatggaagatttgcaaaatacaaatattttacccatgtcatgatcaataagacagatatgctaatgataaccagacgtggtgtattgtttgtaacaaagggaacatttggacaactcacgtgtgagtggcagtatagttttgatgaatttaccaaagagccattcattgttcatgggagaagattgcgcattgaagcaaaggaacgagtgaagtctgtatttcatgccagagagtttggaaaaataattaacttcaagaccccagaggatgccaggtggatcctcacaaagctacaagaagcaagagaaccttctccgagcctctga

SEQ ID NO: 17 - protein, 3135 aa, Homo sapiens
MVFESVVVDVLNRFLGDYVVDLDTSQLSLGIWKGAVALKNLQIKENALSQLDVPFKVKVGHIGNLKLIIPWKNLYTQPVEAVLEEIYLLIVPSSRIKYDPLKEEKQLMEAKQQELKRIEEAKQKVVDQEQHLPEKQDTFAEKLVTQIIKNLQVKISSIHIRYEDDITNRDKPLSFGISLQNLSMQTTDQYWVPCLHDETEKLVRKLIRLDNLFAYWNVKSQMFYLSDYDNSLDDLKNGIVNENIVPEGYDFVFRPISANAKLVMNRRSDFDFSAPKINLEIELHNIAIEFNKPQYFSIMELLESVDMMAQNLPYRKFKPDVPLHHHAREWWAYAIHGVLEVNVCPRLWMWSWKHIRKHRQKVKQYKELYKKKLTSKKPPGELLVSLEELEKTLDVFNITIARQTAEVEVKKAGYKIYKEGVKDPEDNKGWFSWLWSWSEQNTNEQQPDVQPETLEEMLTPEEKALLYEAIGYSETAVDPTLLKTFEALKFFVHLKSMSIVLRENHQKPELVDIVIEEFSTLIVQRPGAQAIKFETKIDSFHITGLPDNSEKPRLLSSLDDAMSLFQITFEINPLDETVSQRCIIEAEPLEIIYDARTVNSIVEFFRPPKEVHLAQLTAATLTKLEEFRSKTATGLLYIIETQKVLDLKINLKASYIIVPQDGIFSPTSNLLLLDLGHLKVTSKSRSELPDVKQGEANLKEIMDRAYDSFDIQLTSVQLLYSRVGDNWREARKLSVSTQHILVPMHFNLELSKAMVFMDVRMPKFKIYGKLPLISLRISDKKLQGIMELIESIPKPEPVTEVSAPVKSFQIQTSTSLGTSQISQKIIPLLELPSVSEDDSEEEFFDAPCSPLEEPLQFPTGVKSIRTRKLQKQDCSVNMTTFKIRFEVPKVLIEFYHLVGDCELSVVEILVLGLGAEIEIRTYDLKANAFLKEFCLKCPEYLDENKKPVYLVTTLDNTMEDLLTLEYVKAEKNVPDLKSTYNNVLQLIKVNFSSLDIHLHTEALLNTINYLHNILPQSEEKSAPVSTTETEDKGDVIKKLGLDSEMIMRPSETEINAKLRNIIVLDSDITAIYKKAVYITGKEVFSFKMVSYMDATAGSAYTDMNVVDIQVNLIVGCIEVVFVTKFLYSILAFIDNFQAAKQALAEATVQAAGMAATGVKELAQRSSRMALDINIKAPVVVIPQSPVSENVFVADFGLITMTNTFHMITESQSSPPPVIDLITIKLSEMRLYRSRFINDAYQEVLDLLLPLNLEVVVERNLCWEWYQEVPCFNVNAQLKPMEFILSQEDITTIFKTLHGNIWYEKDGSASPAVTKDQYSATSGVTTNASHHSGGATVVTAAVVEVHSRALLVKTTLNISFKTDDLTMVLYSPGPKQASFTDVRDPSLKLAEFKLENIISTLKMYTDGSTFSSFSLKNCILDDKRPHVKKATPRMIGLTVGFDKKDMMDIKYRKVRDGCVTDAVFQEMYICASVEFLQTVANVFLEAYTTGTAVETSVQTWTAKEEVPTQESVKWEINVIIKNPEIVFVADMTKNDAPALVITTQCEICYKGNLENSTMTAAIKDLQVRACPFLPVKRKGKITTVLQPCDLFYQTTQKGTDPQVIDMSVKSLTLKVSPVIINTMITITSALYTTKETIPEETASSTAHLWEKKDTKTLKMWFLEESNETEKIAPTTELVPKGEMIKMNIDSIFIVLEAGIGHRTVPMLLAKSRFSGEGKNWSSLINLHCQLELEVHYYNEMFGVWEPLLEPLEIDQTEDFRPWNLGIKMKKKAKMAIVESDPEEENYKVPEYKTVISFHSKDQLNITLSKCGLVMLNNLVKAFTEAATGSSADFVKDLAPFMILNSLGLTISVSPSDSFSVLNIPMAKSYVLKNGESLSMDYIRTKDNDHFNAMTSLSSKLFFILLTPVNHSTADKIPLTKVGRRLYTVRHRESGVERSIVCQIDTVEGSKKVTIRSPVQIRNHFSVPLSVYEGDTLLGTASPENEFNIPLGSYRSFIFLKPEDENYQMCEGIDFEEIIKNDGALLKKKCRSKNPSKESFLINIVPEKDNLTSLSVYSEDGWDLPYIMHLWPPILLRNLLPYKIAYYIEGIENSVFTLSEGHSAQICTAQLGKARLHLKLLDYLNHDWKSEYHIKPNQQDISFVSFTCVTEMEKTDLDIAVHMTYNTGQTVVAFHSPYWMVNKTGRMLQYKADGIHRKHPPNYKKPVLFSFQPNHFFNNNKVQLMVTDSELSNQFSIDTVGSHGAVKCKGLKMDYQVGVTIDLSSFNITRIVTFTPFYMIKNKSKYHISVAEEGNDKWLSLDLEQCIPFWPEYASSKLLIQVERSEDPPKRIYFNKQENCILLRLDNELGGIIAEVNLAEHSTVITFLDYHDGAATFLLINHTKNELVQYNQSSLSEIEDSLPPGKAVFYTWADPVGSRRLKWRCRKSHGEVTQKDDMMMPIDLGEKTIYLVSFFEGLQRIILFTEDPRVFKVTYESEKAELAEQEIAVALQDVGISLVNNYTKQEVAYIGITSSDVVWETKPKKKARWKPMSVKHTEKLEREFKEYTESSPSEDKVIQLDTNVPVRLTPTGHNMKILQPHVIALRRNYLPALKVEYNTSAHQSSFRIQIYRIQIQNQIHGAVFPFVFYPVKPPKSVTMDSAPKPFTDVSIVMRSAGHSQISRIKYFKVLIQEMDLRLDLGFIYALTDLMTEAEVTENTEVELFHKDIEAFKEEYKTASLVDQSQVSLYEYFHISPIKLHLSVSLSSGREEAKDSKQNGGLIPVHSLNLLLKSIGATLTDVQDVVFKLAFFELNYQFHTTSDLQSEVIRHYSKQAIKQMYVLILGLDVLGNPFGLIREFSEGVEAFFYEPYQGAIQGPEEFVEGMALGLKALVGGAVGGLAGAASKITGAMAKGVAAMTMDEDYQQKRREAMNKQPAGFREGITRGGKGLVSGFVSGITGIVTKPIKGAQKGGAAGFFKGVGKGLVGAVARPTGGIIDMASSTFQGIKRATETSEVESLRPPRFFNEDGVIRPYRLRDGTGNQMLQVMENGRFAKYKYFTHVMINKTDMLMITRRGVLFVTKGTFGQLTCEWQYSFDEFTKEPFIVHGRRLRIEAKERVKSVFHAREFGKIINFKTPEDARWILTKLQEAREPSPSL

SEQ ID NO: 18 - protein, 221 aa, Homo sapiens
MFHGIPATPGIGAPGNKPELYEEVKLYKNAREREKYDNMAELFAVVKTMQALEKAYIKDCVSPSEYTAACSRLLVQYKAAFRQVQGSEISSIDEFCRKFRLDCPLAMERIKEDRPITIKDDKGNLNRCIADVVSLFITVMDKLRLEIRAMDEIQPDLRELMETMHRMSHLPPDFEGRQTVSQWLQTLSGMSASDELDDSQVRQMLFDLESAYNAFNRFLHA

SEQ ID NO: 19 - DNA, 666 bp, Homo sapiens
atgtttcatgggatcccagccacgccgggcataggagcccctgggaacaagccggagctgtatgaggaagtgaagttgtacaagaacgcccgggagagggagaagtacgacaacatggcagagctgtttgcggtggtgaagacaatgcaagccctggagaaggcctacatcaaggactgtgtctcccccagcgagtacactgcagcctgctcccggctcctggtccaatacaaagctgccttcaggcaggtccagggctcagaaatcagctctattgacgaattctgccgcaagttccgcctggactgcccgctggccatggagcggatcaaggaggaccggcccatcaccatcaaggacgacaagggcaacctcaaccgctgcatcgcagacgtggtctcgctcttcatcacggtcatggacaagctgcgcctggagatccgcgccatggatgagatccagcccgacctgcgagagctgatggagaccatgcaccgcatgagccacctcccacccgactttgagggccgccagacggtcagccagtggctgcagaccctgagcggcatgtcggcgtcagatgagctggacgactcacaggtgcgtcagatgctgttcgacctggagtcagcctacaacgccttcaaccgcttcctgcatgcctga